(19) United States
(12) Patent Application Publication
(10) Pub. No.: US 2024/0271130 A1
(43) Pub. Date: Aug. 15, 2024

(54) LIPOPHILIC SIRNA CONJUGATES FOR THE TREATMENT OF INFLAMMATORY DISEASES

(71) Applicant: Vanderbilt University, Nashville, TN (US)

(72) Inventors: Craig L. Duvall, Nashville, TN (US); Juan M. Colazo, Nashville, TN (US)

(21) Appl. No.: 18/177,068

(22) Filed: Mar. 1, 2023

Related U.S. Application Data
(60) Provisional application No. 63/484,459, filed on Feb. 10, 2023.

(51) Int. Cl. C12N 15/113; A61P 29/00

(52) U.S. Cl. CPC C12N 15/113; A61P 29/00; C12N 2310/315; C12N 2310/3515

(57) ABSTRACT

Disclosed herein are methods of treating inflammatory diseases, such as arthritis or traumatic injury, using lipophilic siRNA conjugates that can bind to albumin. An example method includes administering to a subject an effective amount of a conjugate, optionally in combination with a pharmaceutically acceptable excipient, wherein the conjugate includes a siRNA capable of inhibiting expression of a protein associated with the inflammatory disease; a lipophilic ligand capable of binding albumin; and a linker attaching the siRNA to the lipophilic ligand, the linker including a branching molecule attached to the siRNA, and a hydrophilic spacer attaching the branching molecule to the lipophilic ligand.

### CROSS-REFERENCE TO RELATED APPLICATIONS

This application claims priority to U.S. Provisional Patent Application No. 63/484,459 filed on Feb. 10, 2023, which is incorporated by reference herein in its entirety.

### STATEMENT REGARDING FEDERALLY SPONSORED RESEARCH

This invention was made with government support under Grant No. R21 AR078636 awarded by the National Institutes of Health. The government has certain rights in the invention.

### REFERENCE TO AN ELECTRONIC SEQUENCE LISTING

The contents of the electronic sequence listing (titled 093386-0004-US02 Sequence Listing.xml; 19,400 bytes; and created on Sep. 19, 2023) is herein incorporated by reference in its entirety.

### TECHNICAL FIELD

The present disclosure relates to lipophilic siRNA conjugates and their use in the treatment of inflammatory diseases.

### INTRODUCTION

Improved efficacy of therapies can be useful in treating diseases, such as inflammatory diseases.

### SUMMARY

In one aspect, disclosed are methods of treating an inflammatory disease in a subject in need thereof, the method including administering to the subject an effective amount of a conjugate, optionally in combination with a pharmaceutically acceptable excipient, wherein the conjugate includes a siRNA capable of inhibiting expression of a protein associated with the inflammatory disease; a lipophilic ligand capable of binding albumin; and a linker attaching the siRNA to the lipophilic ligand, the linker including a branching molecule attached to the siRNA, and a hydrophilic spacer attaching the branching molecule to the lipophilic ligand.

In another aspect, disclosed is a conjugate including a siRNA capable of inhibiting expression of a protein associated with the inflammatory disease; a lipophilic ligand capable of binding albumin; and a linker attaching the siRNA to the lipophilic ligand, the linker including a branching molecule attached to the siRNA, and a hydrophilic spacer attaching the branching molecule to the lipophilic ligand.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1A-1F show stability and matrix metalloproteinase 13 (MMP13) silencing activity of example therapeutic siRNA molecules. FIG. 1A: example modifications that can be used for a siRNA therapeutic including sequence, backbone, and termini (not a comprehensive list of modifications and/or modification patterns that can be used in the disclosed conjugates). FIG. 1B: control si-EG₄₅<L₂ albumin “hitchhiking” molecule, control si-EG₃-cholesterol molecule, and si<(EG₁₈L)₂ molecule. FIG. 1C: comparison of siRNA stability in synovial fluid (human, active osteoarthritis (OA) diagnosis, on no medications time of knee joint aspiration) and serum (60% Fetal Bovine Serum (FBS)) at 37° C. between unmodified and ‘zipper’ modified candidate sequence against mouse MMP13, siMMP13.1, over the course of 24 hours. FIG. 1D: in-vitro carrier-mediated (lipofectamine) knockdown of murine MMP13 in chondrogenic ATDC5 cells after stimulation with TNFα (20 ng/mL). FIG. 1E: si<(EG₁₈L)₂ conjugate in vitro, carrier-free, binding to albumin in OA and RA human synovial fluid to a greater amount than in healthy human synovial fluid. FIG. 1F: si<(EG₁₈L)₂ conjugate in vitro, carrier-free, silencing knockdown of murine MMP13. *P<0.05, **P<0.01, ***P<0.001, ****P<0.0001.

FIG. 2A-2I show knee joint pharmacokinetics of albumin binding Evans blue dye, fluorescently conjugated albumin, and albumin-hitchhiking siRNA<(EG₁₈L)₂ having preferential delivery to loaded, arthritic knee joints. FIG. 2A: unilateral (left knee loaded, right knee unloaded) mechanical loading protocol/timeline used and injection protocol for Evans blue, Cy5-conjugated mouse serum albumin (MSA-Cy5), and systemic Cy5-conjugated siRNA molecules. FIG. 2B: representative intravital IVIS image of a unilaterally loaded mouse injected with Evans blue dye. FIG. 2C: representative intravital IVIS images of a unilaterally loaded mouse injected with Cy5-conjugated mouse serum albumin (MSA-Cy5). FIG. 2D: representative intravital IVIS images of a unilaterally loaded mouse injected with 1 mg/kg Cy5-conjugated siRNA molecules. FIG. 2E: quantification of ex-vivo average radiance in healthy vs. loaded (+OA) knees. FIG. 2F: representative cryohistology (20X) of siRNA<(EG₁₈L)₂ within the arthritic knee and the healthy knee; the cartilage-synovial tissue interface is shown. FIG. 2G: representative cryohistology images (20X) of sagittal-sectioned knee joints with a specific focus on cartilage and synovial tissue (Blue=DAPI, Red=Cy5-conjugated siRNA). FIG. 2H: quantification of ng Evans Blue dye/mg of knee joint tissue (absorbance reading at 610 nm). FIG. 2I: representative images of harvested hindlimb tissue of mice injected with Evans blue dye (black arrows pointing at the mechanically loaded knee joint). *P<0.05, **P<0.01, ***P<0.001, ****P<0.0001.

FIG. 3A-3G show MMP13 knockdown potential in mechanically-loaded post-traumatic osteoarthritis (PTOA) joints for siMMP13<(EG₁₈L)₂. FIG. 3A: mechanical loading (bilateral knees) and injection protocol for longevity of action study. FIG. 3B: knee joint MMP13 knockdown longevity from a single I.V. injection of 10 mg/kg siMMP13<(EG₁₈L)₂ determined by qRT-PCR. FIG. 3C: immunohistochemical staining of MMP13+ regions (brown) of the untreated (top) cartilage, synovium, and meniscus and showing reduced MMP13 protein levels when treated with siMMP13<(EG₁₈L)₂ (bottom). FIG. 3D: representative IVIS images of healthy and PTOA (Loading was performed for 1 week in the left knee only, 3 loading sessions, before injection) mouse knee joints over 30 days after a single 10 mg/kg intravenous (i.v.) injection of Cy5-siRNA<(EG₁₈L)₂. D) Semiquantitative analysis of the AUC based on the fluorescence intensity profiles in (C) (n=4). FIG. 3E: unsupervised sorting of treatment groups from most to least similar to normal joints as quantified by nanoString at day 10 after treatment with 10 mg/kg siMMP13<(EG₁₈L)₂. Gene expression is shown as high-(green) or low-expression (red) sorted vertically by differences between treatment groups. FIG. 3F: Expression of genes clusters associated with the p38/MAPK and TNF-alpha NF-kB signaling pathways. FIG. 3G: chemokine and I1-1 signaling pathways. *P<0.05, **P<0.01, ***P<0.001, ****P<0.0001.

FIG. 4A-4G show protection of mechanically loaded joints from OA progression and increased joint pressure sensitization in PTOA mice treated with siMMP13<(EG₁₈L)₂. FIG. 4A: loading (bilateral) and treatment regimen used in the long-term PTOA mouse model. FIG. 4B: blinded Smalgo algometer readings of mechanical hyperalgesia of the knee joints over time (plotted as percent change over time). FIG. 4C: quantification of total MMP activity in mouse knee joints as determined by MMPsense 750 Fast. FIG. 4D: quantification of the binding of fluorescently labeled mAbCII in the knee joint as a marker for relative cartilage damage and resultant exposure of CII. FIG. 4E: representative Toluidine Blue staining of the articular surface of the femur. FIG. 4F: quantification of cartilage damage by the OARSI osteoarthritis cartilage histopathology assessment system. FIG. 4G: immunohistochemical staining of MMP13+ regions (brown) of the siNEG<(EG₁₈L)₂ (left) cartilage, synovium, and meniscus and showing reduced MMP13 protein levels when treated with siMMP13<(EG₁₈L)₂ (right). *P<0.05, **P<0.01, ***P<0.001, ****P<0.0001.

FIG. 5A-5H show reduction of cartilage degradation products and whole knee joint protection by reducing synovial thickening, osteophyte formation, and pathologic mineralization with siMMP13<(EG₁₈L) treatment. FIG. 5A: Hematoxylin & Eosin (H&E) staining of knee joints, focusing on the synovial regions. FIG. 5B: degenerative joint disease (DJD) score by treatment group; blinded histological scoring was completed by a treatment-blinded histopathologist. FIG. 5C: 3-dimensional (3D) renderings of meniscal/ectopic mineralization and osteophyte growth in the joint space from microCT scan regions in a healthy control (left), and PTOA mice treated with siMMP13<(EG₁₈L)₂ (middle) or siNEG<(EG₁₈L)₂ (right). FIG. 5D: cross-sectional microCT views used to measure osteophyte outgrowth size in a healthy control (left), and PTOA mice treated with siMMP13<(EG₁₈L)₂ (middle) or siNEG<(EG₁₈L)₂ (right). FIG. 5E: measurements of both femoral and tibial osteophyte size at largest outgrowth from normal cortical bone structure. FIG. 5F: quantified amount of ectopic mineralization [hydroxyapatite (HA)] in the menisci and in the form of osteophytes. FIG. 5G: relative serum C2C collagen 2 degradation fragment concentration. FIG. 5H: immunohistochemical staining of C1,2C collagen 2 degradation fragment regions of the siNEG<(EG₁₈L)₂ (left) tibial cartilage and meniscus and showing reduced C1,2C staining levels when treated with siMMP13<(EG₁₈L)₂ (right). *P<0.05, **P<0.01, ***P<0.001, ****P<0.0001.

FIG. 6A-6G show delivery of siMMP13<(EG₁₈L)₂ to multiple joints with treatment provides protection against inflammatory arthritis progression and hyperalgesia. FIG. 6A: representative ex-vivo IVIS images of K/BxN STA mouse limbs injected I.V. with Cy5-conjugated siRNA molecules. FIG. 6B: cryosection of hindpaws shows Cy5-conjugated siRNA<(EG₁₈L)₂ within important homeostatic joint tissues. Albumin-hitchhiking siMMP13<(EG₁₈L)₂ provides protection against clinical score (FIG. 6C), ankle width change (FIG. 6D), severity index (FIG. 6E), and algometer ankle joint hyperalgesia (FIG. 6F) over time. FIG. 6G: representative images of K/BxN STA mice in each group showing severe inflammation in all 4 paws in the no treatment control (black arrows) with a focus on hindpaws. K/BxN serum was injected I.P. on day 0 meanwhile 10 mg/kg of the treatments were injected on day 2; All mice were sacrificed at day 10 after K/BxN serum injection for analyses of joint tissues. *P<0.05, **P<0.01, ***P<0.001, ****P<0.0001.

FIG. 7A-7D show siMMP13<(EG₁₈L)₂ can silence MMP13 expression and provide cartilage protection in multiple joints in a K/BxN STA model. FIG. 7A: representative ex-vivo IVIS images of treated K/BxN STA mouse limbs injected with mAbCII and MMPsense 750 Fast which indicate relative cartilage damage and MMP proteolytic activity, respectively. FIG. 7B: multi-joint quantification of total MMP activity as determined by MMPsense 750 Fast and of the binding of fluorescently labeled mAbCII as a marker for relative cartilage damage and resultant exposure of CII. FIG. 7C: relative MMP13 gene expression in hindpaws (ankle), knees, and forepaws (wrist) determined by qRT-PCR. FIG. 7D: immunohistochemical staining of MMP13+ regions (brown) in multiple joints of the untreated K/BxN STA mice (left) cartilage, synovium, and meniscus/joint tissues; showing reduced MMP13 protein levels when K/BxN mice were treated with siMMP13<(EG₁₈L)₂ (right). K/BxN serum was injected I.P. on day 0 meanwhile 10 mg/kg of the treatments were injected on day 2; all mice were sacrificed at day 10 after K/BxN serum injection for analyses of joint tissues. *P<0.05, **P<0.01, ***P<0.001, ****P<0.0001.

FIG. 8A-8G show siMMP13<(EG₁₈L)₂ treatment can provide protection against cartilage destruction, synovial inflammation, and bone destruction in the K/BxN STA model. FIG. 8A: representative toluidine blue-stained sections of the ankle cartilage within the hindpaw (left) and H&E staining of the hindpaw, focusing on the synovial regions around the ankle cartilage (right). FIG. 8B: cartilage destruction score per treatment group determined by a treatment-blinded histopathologist. FIG. 8C: inflammation score per treatment group scored by a treatment-blinded histopathologist. FIG. 8D: 3D renderings of hindpaws from microCT scans. FIG. 8E: bone fraction (bone volume/total volume; BV/TV). FIG. 8F: bone surface to bone volume (BS/BV). FIG. 8G: bone mineral density (BMD; g/cm³). K/BxN serum was injected I.P. on day 0 meanwhile 10 mg/kg of the treatments were injected on day 2; all mice were sacrificed at day 10 after K/BxN serum injection for analyses of joint tissues. *P<0.05, **P<0.01, ***P<0.001, ****P<0.0001.

FIG. 9A-9E show siMMP13<(EG₁₈L)₂ treatment can reduce cartilage degradation fragments and inflammatory signaling in arthritis hindpaws. FIG. 9A: unsupervised sorting of treatment groups from most to least similar to normal hindpaws as quantified by nanoString at day 10 after treatment with 10 mg/kg siMMP13<(EG₁₈L)₂. Gene expression is shown as high-(green) or low-expression (red) sorted vertically by differences between treatment groups. FIG. 9B: relative Serum C2C Collagen 2 degradation fragment concentration. FIG. 9C: immunohistochemical staining (brown) of C1,2C collagen 2 degradation fragment regions of untreated K/BxN knee and hindpaw cartilage (top) and showing reduced C1,2C staining levels when treated with siMMP13<(EG₁₈L)₂ (bottom). FIG. 9D: expression of gene clusters related to complement and coagulation cascade, I1-1 signaling pathway, and the relationship between inflammation, COX-2, and EGFR. FIG. 9E: I1-18 signaling pathway and chemokine signaling pathway. *P<0.05, **P<0.01, ***P<0.001, ****P<0.0001.

FIG. 10A-10F show siMMP13<(EG₁₈L)₂ can provide effective delivery and MMP13 silencing in a guinea pig anterior cruciate ligament transection (ACLT) large animal model. FIG. 10A: ACLT (unilateral, left knee joint) surgery and treatment regimen used in a Dunkin-Hartley Guinea Pig Model. FIG. 10B: representative ex-vivo IVIS images of unilateral ACLT Guinea Pig knee joint injected with 1 mg/kg Cy5-conjugated siRNA molecules. FIG. 10C: quantification of IVIS osteoarthritic (ACLT) knee joint/healthy knee joint radiant efficiency ratio showing preferential delivery to the injured over normal knees following intravenous delivery. FIG. 10D: representative cryohistology images (20X) of sagittal-sectioned ACLT guinea pig knee joints with a specific focus on cartilage and synovial tissue (Blue=DAPI, Red=Cy5-conjugated siRNA). FIG. 10E: immunofluorescence staining of MMP13 in cartilage/synovium from ACLT knee joints that were untreated (left) or treated with 10 mg/kg siMMP13<(EG₁₈L)₂ (right). FIG. 10F: quantification of MMP13 fluorescence intensity in images of MMP13 immunohistochemistry. *P<0.05, **P<0.01, ***P<0.001, ****P<0.0001.

FIG. 11A-11C show subcutaneous administration of siRNA<(EG₁₈L)₂. FIG. 11A: loading and injection schematic for subcutaneous delivery. FIG. 11B: intravital IVIS image 24 hours after subcutaneous (SubQ) injection of 2 mg/kg Cy5-siRNA<(EG₁₈L)₂ and quantification of Cy5-siRNA<(EG₁₈L)₂ in loaded vs. healthy knee joints from IVIS. FIG. 11C: qRT-PCR analysis of MMP13 mRNA expression in the knee joint using the SubQ route 5 days after injection.

FIG. 12A-12F show intra-articular administration of siRNA<(EG₁₈L)₂. FIG. 12A: loading and injection schematic for intra-articular delivery. FIG. 12B: IVIS image of organs and knee joints 48 hours after intra-articular injection of 0.25 mg/kg Cy5-siRNA<(EG₁₈L)₂ or Cy5-siRNA and quantification. FIG. 12C: cryohistology of the loaded knee joints 48 hours after intra-articular injection with a focus on cartilage and synovial tissues. FIG. 12D: representative IVIS images of healthy and PTOA mouse knee joints over 30 days after a single 1 mg/kg intra-articular (IA) injection of Cy5-siRNA<(EG₁₈L)₂ or Cy5-siRNA. FIG. 12E: semiquantitative analysis of time-course fluorescent radiant efficiency and corresponding AUC within healthy and OA mouse knee joints over 30 days (n=3) (two left plots) and semiquantitative analysis of time-course fraction of retention and corresponding AUC within healthy and OA mouse knee joints over 30 days (n=3) (two right plots). FIG. 12F: qRT-PCR analysis of MMP13 mRNA expression in the knee joint comparing intra-articular 1 mg/kg vs. intravenous 10 mg/kg vs. subcutaneous 20 mg/kg siMMP13<(EG₁₈L)₂ 5 days after injection.

### DETAILED DESCRIPTION

### 1. Definitions

Unless otherwise defined, all technical and scientific terms used herein have the same meaning as commonly understood by one of ordinary skill in the art. In case of conflict, the present document, including definitions, will control. The materials, methods, and examples disclosed herein are illustrative only and not intended to be limiting. Methods and materials similar or equivalent to those described herein can be used in practice or testing of the disclosed invention. All publications, patent applications, patents and other references mentioned herein are incorporated by reference in their entirety.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are contemplated, and for the range 1.5-2, the numbers 1.5, 1.6, 1.7, 1.8, 1.9, and 2 are contemplated.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987; the entire contents of each of which are incorporated herein by reference.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “alkenyl” refers to a straight or branched, unsaturated hydrocarbon chain containing at least one carbon-carbon double bond and from 2 to 10 carbon atoms.

The term “alkyl” refers to a straight or branched, saturated hydrocarbon chain containing from 1 to 10 carbon atoms. The term “C₁-C₃ alkyl” means a straight or branched chain hydrocarbon containing from 1 to 3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

The term “alkynyl” means a straight or branched chain hydrocarbon group containing from 2 to 10 carbon atoms and containing at least one carbon-carbon triple bond. Representative examples of alkynyl include, but are not limited to, acetylenyl, 1-propynyl, 2-propynyl, 3-butynyl, 2-pentynyl, and 1-butynyl.

The term “amino” refers to —NH₂.

The term “antisense strand” refers to a strand of an siRNA duplex that contains some degree of complementarity to a oligonucleotide and contains complementarity to the sense strand of the siRNA duplex.

The term “attached” refers to two moieties being attached through a bond where there can be intervening moieties or molecules in between. For example, the branching molecule can be attached to the siRNA by having an intervening moiety, such as a second linker, between the siRNA and the linker. Attached can also include “directly attached,” which refers to two moieties being attached through a bond with no other intervening moieties or molecules.

The term “carboxyl” refers to the group —C(═O)OR, wherein R is selected from the group consisting of hydrogen, alkyl, alkenyl, and alkynyl, any of which may be optionally substituted, e.g., with one or more substituents.

The term “complementary” refers to the relationship between nucleotides exhibiting Watson-Crick base pairing, or to oligonucleotides that hybridize via Watson-Crick base pairing to form a double-stranded nucleic acid. The term “complementarity” refers to the state of an oligonucleotide (e.g., a sense strand or an antisense strand) that is partially or completely complementary to another oligonucleotide. Oligonucleotides described herein as having complementarity to a target oligonucleotide may be ˜100%, >99%, >95%, >90%, >85%, >80%, >75%, >70%, >65%, >60%, >55% or >50% complementary to the target oligonucleotide. Complementary and complementarity can also be used to describe specifically hybridizing to a target oligonucleotide by a siRNA.

The terms “effective amount” or “therapeutically effective amount” refer to an amount sufficient to effect beneficial or desirable biological and/or clinical results to treat a disease or one or more of its symptoms and/or to prevent or reduce the risk of the occurrence or reoccurrence of the disease or disorder or symptom(s) thereof. In reference to inflammatory diseases an effective or therapeutically effective amount can include an amount sufficient to, among other things, decrease the underlying pathology associated with the inflammatory disease.

The term “hydroxyl” refers to an —OH group.

The term “oligonucleotide” refers to a polymer of nucleotides. The terms “polynucleotide,” “nucleic acid,” and “oligonucleotide”, may be used interchangeably herein. Typically, a polynucleotide comprises at least three nucleotides. Oligonucleotides can be single stranded or double stranded. Example oligonucleotides include, but are not limited to, DNA and RNA, such as mRNA, RNAi, siRNA, and shRNA.

The term “sense strand” refers to a strand of an siRNA duplex that contains complementarity to an antisense strand of the siRNA duplex.

The term “siRNA” refers to small interfering RNAs that induce the RNA interference (RNAi) pathway. siRNA molecules can vary in length (e.g., 10-30 base pairs) and contain varying degrees of complementarity to their target oligonucleotide. The term “siRNA” can include duplexes of two separate strands, as well as single strands that can form hairpin structures comprising a duplex region.

The terms “treatment” or “treating” refer to the medical management of a subject with the intent to heal, cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### 2. Methods

Disclosed herein are methods of treating an inflammatory disease in a subject. The method can include administering to the subject an effective amount of a conjugate. The conjugate can be administered optionally with a pharmaceutically acceptable excipient. The conjugate can include a siRNA capable of inhibiting expression of a protein associated with the inflammatory disease; a lipophilic ligand capable of binding albumin; and a linker attaching the siRNA to the lipophilic ligand, the linker including a branching molecule attached to the siRNA, and a hydrophilic spacer attaching the branching molecule to the lipophilic ligand.

A number of inflammatory diseases may be treated by the disclosed methods. For example, the inflammatory disease can be arthritis, or an inflammatory state associated with a traumatic injury. Examples of arthritis include, but are not limited to, osteoarthritis, rheumatoid arthritis, inflammatory arthritis, multi-joint arthritis, gout, psoriatic arthritis, and septic arthritis. Examples of a traumatic injury include, but are not limited to, bone fracture, ligament tear, tendon tear, and soft tissue injury (e.g., due to a mechanical force). In addition, the methods can be used to treat infections associated with the traumatic injury, such as, but not limited to, osteomyelitis, septic arthritis, and myositis. Genetic disorders that cause inherent inflammation and/or damage to tissues may also be treated.

In some embodiments, the inflammatory disease is arthritis. In some embodiments, arthritis is selected from the group consisting of osteoarthritis, rheumatoid arthritis, inflammatory arthritis, and multi-joint arthritis. In some embodiments, arthritis is selected from the group consisting of osteoarthritis and rheumatoid arthritis. In some embodiments, the inflammatory disease is osteoarthritis.

The subject of the disclosed methods is generally not limited and can be any animal that is in need of a treatment for an inflammatory disease. Subject can mean a mammal that wants or is in need of the herein described conjugates or methods. The subject may be a human or a non-human animal. The subject may be a mammal. The mammal may be a primate or a non-primate. The mammal can be a primate such as a human; a non-primate such as, for example, dog, cat, horse, cow, pig, mouse, rat, camel, llama, goat, rabbit, sheep, hamster, and guinea pig; or non-human primate such as, for example, monkey, chimpanzee, gorilla, orangutan, and gibbon. The subject may be of any age or stage of development, such as, for example, an adult, an adolescent, or an infant. The subject may be male or female. In some embodiments, the subject is human.

The methods can provide advantageous benefits to the subject receiving the treatment. For example, the method can decrease the underlying pathology associated with the inflammatory disease in the subject. Underlying pathology as it relates to the inflammatory disease can include causes, developments, and/or structural/functional changes associated with the inflammatory disease and its progression. For example, for arthritis, decreasing the underlying pathology can include increasing cartilage protection or decreasing synovial inflammation in the subject compared to the subject not receiving the administration or compared to a time point prior to administration. The underlying pathology for individual inflammatory diseases may differ, and it is within the skill of the artisan to recognize different underlying pathologies for different diseases. In some embodiments, the method decreases the underlying pathology associated with the inflammatory disease for at least 50 days post-administration, at least 40 days post-administration, at least 30 days post-administration, at least 25 days post-administration, at least 20 days post-administration, at least 15 days post-administration, or at least 10 days post-administration. In some embodiments, the method decreases the underlying pathology associated with the inflammatory disease for greater than 5 days post-administration, greater than 10 days post-administration, greater than 15 days post-administration, greater than 20 days post-administration, greater than 25 days post-administration, or greater than 30 days post-administration.

The method's benefits to the subject can be assessed through a number of different criteria. For example, following administration, the subject can have decreased hyperalgesia, increased cartilage protection, decreased synovial inflammation, decreased osteophytes, decreased bone erosion, or a combination thereof at a site associated with the inflammatory disease. In some embodiments, the subject has decreased hyperalgesia, increased cartilage protection, decreased synovial inflammation, decreased osteophytes, decreased bone erosion, or a combination thereof in a joint associated with the inflammatory disease following administration.

The joint can be any joint associated with the inflammatory disease. Example joints include, but are not limited to, a knee, a wrist, an ankle, a shoulder, and a spinal joint. In some embodiments, the joint is a knee, a wrist, an ankle, a shoulder, a spinal joint, or a combination thereof. In some embodiments, the joint is a knee, a wrist, an ankle, a shoulder, or a spinal joint. In some embodiments, the joint is a knee, a wrist, an ankle, or a shoulder. In some embodiments, the joint is a knee, a wrist, or an ankle. In addition, a joint can include more than one of that joint if a plurality of joints exists (e.g., more than 1 ankle, more than 1 wrist, etc.).

The method can also outperform control therapies, such as a steroid therapy. For example, the method can decrease the underlying pathology of the inflammatory disease compared to a steroid control. In some embodiments, the method increases cartilage protection compared to a steroid control. Example steroid therapies include, but are not limited to, Zilretta, dexamethosone, cortisone, predinsolone, methylprednisolone, and triamcinolone. In some embodiments, the steroid control is Zilretta or methylprednisolone. In some embodiments, the steroid control is Zilretta.

Numerous techniques known within the art can be used to assess the associated benefits of the disclosed methods, such as gene or protein expression of target molecules and histology of a site associated with the inflammatory disease. Further discussion of assessing the disclosed methods can be found herein.

### A. Conjugates

The conjugate can provide advantageous benefits for the disclosed methods. For example, the conjugate, following administration, can localize or be retained at a site of inflammation associated with the inflammatory disease. This can allow for a more targeted treatment of the inflammatory disease, while decreasing potential side-effects at non-inflammatory sites within the subject.

The conjugate includes a siRNA, a lipophilic ligand capable of binding albumin, and a linker attaching the siRNA and the lipophilic ligand. The linker includes a branching molecule attached to the siRNA and a hydrophilic spacer attaching the branching molecule to the lipophilic ligand.

The siRNA, the linker, and the lipophilic ligand can be attached to each other through various types of linkages/bonds. For example, the siRNA, the linker, and/or the lipophilic ligand can be attached through phosphorothioate bonds, phosphodiester bonds, a cleavable linker (e.g., deoxythymidine (dT), pH-cleavable bond such as ketal), or a combination thereof. In some embodiments, the siRNA, the linker, and/or the lipophilic ligand are attached through phosphorothioate bonds. In some embodiments, the conjugate includes about 20% to about 60% phosphorothioate linkages based on the total amount of phosphate-based linkages in the conjugate, such as about 20% to about 55% phosphorothioate linkages, about 25% to about 50% phosphorothioate linkages, about 35% to about 45% phosphorothioate linkages, or about 40% to about 45% phosphorothioate linkages—based on the total amount of phosphate-based linkages in the conjugate. The combination of phosphodiester linkages and phosphorothioate linkages can be referred to as the total amount of phosphate-based linkages, and is not inclusive of potential phosphorylation of sequences, e.g., to avoid deactivation of phosphatases.

The conjugate's arrangement and composition can provide advantageous benefits, such as being able to bind albumin while also minimizing its propensity to self-assemble into micelles. For example, the conjugate can have a binding affinity (K_{d}) to albumin of less than 1 μM, less than 500 nM, less than 250 nM, less than 100 nM, less than 80 nM, less than 60 nM, less than 50 nM, less than 45 nM, less than 40 nM, or less than 35 nM. In some embodiments, the conjugate has a K_{d} to albumin of greater than 0.1 nM, greater than 0.2 nM, greater than 0.4 nM, greater than 0.5 nM, greater than 0.6 nM, greater than 0.7 nM, greater than 0.8 nM, greater than 0.9 nM, greater than 1 nM, or greater than 5 nM. In some embodiments, the conjugate has a K_{d} to albumin of about 0.1 nM to about 1 μM, such as about 0.5 nM to about 500 nM, about 0.8 nM to about 100 nM, about 0.5 nM to about 100 nM, about 1 nM to about 50 nM, or about 5 nM to about 35 nM. The conjugate can reversibly bind albumin. In some embodiments, the conjugate does not covalently bind to albumin.

In addition, the conjugate can have a critical micelle concentration of greater than 1850 nM, greater than 1900 nM, greater than 1950 nM, greater than 2000 nM, greater than 2100 nM, greater than 2200 nM, greater than 2300 nM, greater than 2400 nM, greater than 2500 nM, greater than 3000 nM, or greater than 3500 nM. In some embodiments, the conjugate has a critical micelle concentration of less than 4500 nM, less than 4000 nM, less than 3500 nM, less than 3000 nM, less than 2500 nM, less than 2400 nM, less than 2300 nM, less than 2200 nM, less than 2100 nM, less than 2000 nM, or less than 1950 nM. In some embodiments, the conjugate has a critical micelle concentration of about 1850 nM to about 4000 nM, such as about 1900 nM to about 3500 nM, about 2000 nM to about 3500 nM, about 1850 nM to about 3500 nM, or about 2500 nM to about 3500 nM.

As discussed elsewhere, the conjugate can bind albumin through the lipophilic ligand. In some embodiments, the binding of the conjugate to the serum protein albumin enhances the pharmacokinetic properties of the siRNA as compared to an unmodified siRNA and/or existing nanocarrier including the siRNA. In some embodiments, enhancing the pharmacokinetic properties includes increasing the circulation half-life and/or bioavailability of the siRNA, as compared to an unmodified siRNA and/or existing nanocarrier including the siRNA. Additionally, enhancing the pharmacokinetic properties may include increasing the quantity of cellular accumulation, increasing the homogeneity of cellular accumulation, increasing resistance to nucleases, and/or permitting increased dosing amount with decreased toxicity as compared to an unmodified siRNA and/or existing nanocarrier including the siRNA.

### i. siRNA

The conjugate includes a siRNA. The siRNA can instill a therapeutic and/or beneficial property to the conjugate. The siRNA can be single stranded or double stranded. An example of a single stranded siRNA includes, but is not limited to, single stranded antisense RNA.

The benefits realized from the end modification of the disclosed conjugate (e.g., attaching the lipophilic ligand to the siRNA through the linker) can be used with any desirable siRNA useful for treatment of an inflammatory disease. In other words, the siRNA of the conjugate is sequence agnostic. This modification can be added to either single or double stranded siRNA that contain either natural or modified nucleotide bases. The single or double stranded nucleotides can also be of variable length, such as 10 to 40 bases in length.

For an example process of selecting an siRNA, the sequence can be first determined using publicly available prediction algorithms. These algorithms can generate many candidate sequences for targeting any given gene. These potential sequences are first screened for on-target gene silencing potency in vitro (without chemical modifications). After identification of one or more potent sequences, chemical modifications can be added to the sequence, and it can be rescreened for in vitro gene silencing activity prior to screening for albumin binding affinity and pharmacokinetic/pharmacodynamic behaviors in vivo. The disclosed albumin binding end chemistry may be successfully integrated with multiple sequences targeting any single gene and may also be adapted for delivery of sequences against theoretically any gene of interest.

The siRNA can be capable of specifically hybridizing to an oligonucleotide encoding a protein associated with a signaling pathway involved with the pathology of the inflammatory disease. For example, the siRNA can be capable of specifically hybridizing to an oligonucleotide encoding a protein of a p38/MAPK signaling pathway, a TNF-α NF-κB signaling pathway, a chemokine signaling pathway, an IL-1 signaling pathway, a senescence-associated secretory phenotype (SASP) pathway, or combinations thereof. In some embodiments, the siRNA is capable of specifically hybridizing to an oligonucleotide encoding a protein of a p38/MAPK signaling pathway, a TNF-α NF-κB signaling pathway, a chemokine signaling pathway, an IL-1 signaling pathway, a SASP pathway, or a combination thereof. In some embodiments, the siRNA is capable of specifically hybridizing to an oligonucleotide encoding a protein of a p38/MAPK signaling pathway, a TNF-α NF-κB signaling pathway, a chemokine signaling pathway, an IL-1 signaling pathway, or a SASP pathway. In some embodiments, the siRNA is capable of specifically hybridizing to an oligonucleotide encoding a protein of a p38/MAPK signaling pathway, a TNF-α NF-κB signaling pathway, a chemokine signaling pathway, or an IL-1 signaling pathway.

The siRNA may be capable of specifically hybridizing to an oligonucleotide encoding an extracellular matrix degrading protein. Examples of extracellular matrix degrading proteins include, but are not limited to, MMPs, ADAMs, and ADAMTS. In some embodiments, the siRNA is capable of specifically hybridizing to an oligonucleotide encoding an MMP, an ADAM, or an ADAMTS.

In some embodiments, the siRNA is capable of specifically hybridizing to an oligonucleotide encoding MMP13, Cadherin-11, MMP1, SOX5, NGF, or MK2. In some embodiments, the siRNA is capable of specifically hybridizing to an oligonucleotide encoding MMP13.

The siRNA can include a plurality of stabilizing modifications. Examples of stabilizing modifications include, but are not limited to, phosphorothioate linkages, 2′F modification, 2′OMe modification, and combinations of 2′F and 2′OMe modifications (e.g., zipper pattern). In addition, the siRNA can include both phosphodiester linkages and phosphorothioate linkages. In some embodiments, the siRNA includes a plurality of phosphorothioate linkages. In some embodiments, the siRNA includes phosphorothioate linkages at its terminal end(s). In some embodiments, the siRNA includes about 1% to about 30% phosphorothioate linkages based on a total amount of phosphate-based linkages in the siRNA, such as about 10% to about 25% phosphorothioate linkages or about 15% to about 24% phosphorothioate linkages—based on a total amount of phosphate-based linkages in the siRNA. In some embodiments, the siRNA includes about 21% phosphorothioate linkages based on a total amount of phosphate-based linkages in the siRNA.

The siRNA can have a varying amount of nucleotides. For example, the siRNA can have about 15 to about 40 nucleotides, such as about 16 to about 38 nucleotides, about 15 to about 35 nucleotides, about 18 to about 32 nucleotides, about 18 to about 30 nucleotides, or about 20 to about 35 nucleotides.

In some embodiments, the siRNA includes a nucleotide sequence of SEQ ID NO: 1, SEQ ID NO: 2, SEQ ID NO: 3, SEQ ID NO: 4, or a combination thereof. In some embodiments, the siRNA includes a nucleotide sequence of SEQ ID NO: 1, SEQ ID NO: 2, or a combination thereof. In some embodiments, the siRNA includes a nucleotide sequence of SEQ ID NO: 3, SEQ ID NO: 4, or a combination thereof.

### ii. Lipophilic Ligand

The lipophilic ligand is capable of binding albumin, and thus can instill in the conjugate the ability to bind albumin. The lipophilic ligand can include any lipophilic moiety suitable for binding into a fatty acid pocket of albumin. In some embodiments, the lipophilic ligand includes a lipid with a long hydrocarbon chain.

The lipid can include a C₁₂-C₂₂ hydrocarbon chain, such as a C₁₂-C₂₀ hydrocarbon chain, a C₁₄-C₂₂ hydrocarbon chain, a C₁₆-C₂₂ hydrocarbon chain, or a C₁₆-C₂₀ hydrocarbon chain. In some embodiments, the lipid includes a C₁₈ hydrocarbon chain. The lipid can be saturated or unsaturated. In addition, the lipid may have a terminal end. The terminal end may include a functional group that may aid in binding. In some embodiments, the terminal end of the lipid includes an alkyl, carboxyl, hydroxyl, or amino. In some embodiments, the terminal end of the lipid includes an alkyl or carboxyl. In embodiments where there is more than one lipid, each lipid can include a different functional group at its terminal end or can include the same functional group. For example, one terminal end can include an alkyl and one terminal end can include a carboxyl, or both terminal ends can include, e.g., an alkyl. In some embodiments, the terminal end includes an alkyl. In some embodiments, the terminal end of the lipid does not include a hydroxyl or a carboxyl.

The lipophilic ligand can include more than one lipid. Having more than one lipid can allow for multivalency of the lipophilic ligand and the conjugate thereof. The lipophilic ligand can include at least 2 lipids, at least 3 lipids, at least 4 lipids, at least 5 lipids, at least 6 lipids, at least 7 lipids, or at least 8 lipids. In some embodiments, the lipophilic ligand includes less than 10 lipids, less than 9 lipids, less than 8 lipids, less than 7 lipids, less than 6 lipids, or less than 5 lipids. In some embodiments, the lipophilic ligand includes 1 to 10 lipids, such as 1 to 8 lipids, 1 to 6 lipids, 2 to 8 lipids, 2 to 6 lipids, 2 to 4 lipids, or 2 to 3 lipids.

The lipophilic ligand can be divalent. For example, the lipophilic ligand can include two independent lipids. In some embodiments, the lipophilic ligand incudes two independent lipids, each lipid including a C₁₂-C₂₂ hydrocarbon chain. In some embodiments, the lipophilic ligand includes two independent lipids, each lipid including a C₁₈ hydrocarbon chain.

The lipophilic ligand and lipid(s) can be attached to the hydrophilic spacer. In some embodiments, the lipophilic ligand and lipid(s) are directly attached to the hydrophilic spacer. In some embodiments, the lipophilic ligand includes two individual lipids, each lipid bound to a separate, individual hydrophilic spacer which is bound to a separate branch of the branching molecule. In such embodiments, the lipids may be the same or different. For example, the lipids can both include a C₁₈ hydrocarbon chain. Alternatively, in other embodiments, each lipid can include a hydrocarbon chain of varying length. In some embodiments, the lipophilic ligand includes two distinct types of lipids.

### iii. Linker

The lipophilic ligand is attached to the siRNA through a linker. The arrangement and composition of the linker can provide the conjugate with advantageous properties, such as, but not limited to, binding to albumin, decreased propensity to self-assembly into micelles, and improved pharmacokinetics. The linker includes a branching molecule and a hydrophilic spacer. The linker can further include other types of spacers and/or linkers known within the art.

### a. Branching Molecule

The branching molecule can be any suitable molecule that allows for branching of the conjugate, e.g., extending from the siRNA. Example branching molecules include, but are not limited to, a phosphoramidite (e.g., symmetrical branching CED phosphoramidite), a tri-valent splitter, or a tetra-valent splitter.

The branching molecule can be positioned between the siRNA and the hydrophilic spacer. Or in other words, the branching molecule can be attached to the siRNA and the hydrophilic spacer. The branching molecule can also be directly attached to the siRNA. In some embodiments, the branching molecule is directly attached to the siRNA and attached to the hydrophilic spacer. In some embodiments, the branching molecule is directly attached to the siRNA and directly attached to the hydrophilic spacer. The branching molecule can be directly attached, or conjugated, to the siRNA through a phosphorothioate bond, phosphodiester, or cleavable linker (e.g., deoxythymidine (dT), pH-cleavable bond such as ketal). Although, in some embodiments, there are no intervening moieties or molecules when directly attached, as will be appreciated by those skilled in the art, the placement of the branch point within the branching molecule may be adjusted based upon the structure of the branching molecule itself.

The branching molecule can have multiple independent branch points, each branch point having at least two independent branches. For example, the branching molecule can have at least 2 branch points, at least 3 branch points, at least 4 branch points, or at least 5 branch points. In some embodiments, the branching molecule has less than 7 branch points, less than 6 branch points, less than 5 branch points, or less than 4 branch points. In some embodiments, the branching molecule has 1 to 5 branch points, such as 1 to 4 branch points, 1 to 3 branch points, or 1 to 2 branch points.

Each branch point can have multiple, independent branches. For example, each branch point can have at least 2 branches, at least 3 branches, at least 4 branches, at least 5 branches, at least 6 branches, at least 7 branches, at least 8 branches, at least 9 branches, or at least 10 branches. In some embodiments, each branch point has less than 12 branches, less than 11 branches, less than 10 branches, less than 9 branches, less than 8 branches, less than 7 branches, less than 6 branches, less than 5 branches, or less than 4 branches. In some embodiments, each branch point has 2 to 12 branches, such as 2 to 10 branches, 2 to 8 branches, 2 to 6 branches, or 2 to 4 branches. In some embodiments, each branch point has 2 branches.

The branching molecule's positioning in the conjugate can play an important role in determining properties of the overall conjugate. For example, attaching the branching molecule to the siRNA, rather than to the lipophilic ligand, and having the hydrophilic spacer between the branching molecule and the lipophilic ligand unexpectedly provides improved properties. It is hypothesized, without wishing to be bound by a particular theory, that the positioning of the hydrophilic spacer after the branching molecule can provide additional flexibility and separation between the lipophilic ligand and lipid(s) thereof. The lipophilic ligand and lipid(s) thereof with this additional flexibility and separation show higher affinity for albumin. Additionally, and again without wishing to be bound by a particular theory, it is hypothesized that attaching the branching molecule to the siRNA can decrease the self-micellization of the conjugate, which in turn can allow the conjugate to remain more unimeric in solution, and thus can be more available for binding to albumin. In addition, this may aid in binding to the outer surface of a cell membrane, which can promote internalization. In contrast, when the hydrophilic spacer is positioned before the branching molecule the lipophilic ligand and lipid(s) thereof can be more closely spaced, which can cause the conjugate to self-assemble, e.g., into a micelle, thereby limiting the lipophilic ligand's ability to interact with albumin.

The branching molecule can be included in the conjugate as a way to introduce multifunctionality, such as multivalency, to the conjugate. For example, the branching molecule can be used to increase the valency of the lipophilic ligand and the conjugate. The branching molecule can have independent branches that are attached to independent lipids. In some embodiments, the branching molecule has 2 independent branches that are attached to 2 independent lipids. The same can be said if there are 3 independent branches, these individual branches can be attached to 3 independent lipids. However, in some embodiments, not every branch is attached to a lipid. For example, in some embodiments, the branching molecule can have 4 branches, where only 2 of the 4 branches are attached to a lipid. Varying combinations of branches and their attachment to lipids can be used for the disclosed conjugate.

### b. Hydrophilic Spacer

The hydrophilic spacer can include any suitable hydrophilic compound for attaching the lipophilic ligand to the branching molecule. Examples of suitable hydrophilic compounds include, but are not limited to, ethylene glycol, zwitterionic linkers, peptoids (e.g., poly(sarcosine)), amino acids, poly(ethylene glycol) substitutes including: poly(glycerols), poly(oxazoline), poly(acrylamide), poly (N-acryloyl morpholine, poly(N,N-dimethyl acrylamide), poly(2-hydroxypropyl methacrylamide), poly(2-hydroxyethyl methacryalmide), and any other similar hydrophilic spacer molecule and/or polymer.

The hydrophilic spacer can be attached to the lipophilic ligand and the branching molecule. As mentioned above, the branching molecule can include a branching point having at least two independent branches. Each branch of the branching molecule can be attached to an individual hydrophilic spacer. In addition, each hydrophilic spacer can be individually attached to an individual lipid of the lipophilic ligand. In some embodiments, the hydrophilic spacer is attached to the lipophilic ligand, the branching molecule, or both through phosphorothioate bonds. In some embodiments, the hydrophilic spacer is attached to a lipid of the lipophilic ligand, the branching molecule, or both through phosphorothioate bonds.

The hydrophilic spacer can include at least one hydrophilic block. For example, the hydrophilic spacer can include 1 to 100 hydrophilic blocks, such as 1 to 50 hydrophilic blocks, 1 to 20 hydrophilic blocks, 1 to 18 hydrophilic blocks, 2 to 15 hydrophilic blocks, 3 to 10 hydrophilic blocks, 2 to 10 hydrophilic blocks, 1 to 15 hydrophilic blocks, 1 to 10 hydrophilic blocks, 2 to 8 hydrophilic blocks, 2 to 6 hydrophilic blocks, or 1 to 7 hydrophilic blocks. In some embodiments, the hydrophilic spacer includes 5 hydrophilic blocks. The hydrophilic blocks can be attached to each other, the branching molecule, and/or the lipophilic ligand. For example, the hydrophilic blocks can be attached to each other, the branching molecule, and/or the lipophilic ligand through phosphorothioate bonds, phosphodiester, or a cleavable linker (e.g., deoxythymidine (dT), pH-cleavable bond such as ketal). In some embodiments, each of the hydrophilic blocks are attached to each other through phosphorothioate linkages.

The hydrophilic block can include repeats of the hydrophilic compound. In some embodiments, the hydrophilic spacer includes 1 to 100 hydrophilic blocks (as described above), with each of the repeating blocks including 1 to 150 repeats of the hydrophilic compound, such as 1 to 100 repeats of the hydrophilic compound, 2 to 50 repeats of the hydrophilic compound, 1 to 45 repeats of the hydrophilic compound, 1 to 30 repeats of the hydrophilic compound, 2 to 20 repeats of the hydrophilic compound, or 2 to 10 repeats of the hydrophilic compound. In some embodiments, each hydrophilic block includes less than 150 repeats of the hydrophilic compound, less than 100 repeats of the hydrophilic compound, less than 75 repeats of the hydrophilic compound, less than 50 repeats of the hydrophilic compound, less than 45 repeats of the hydrophilic compound, less than 40 repeats of the hydrophilic compound, or less than 35 repeats of the hydrophilic compound. In some embodiments, each hydrophilic block includes greater than 2 repeats of the hydrophilic compound, greater than 3 repeats of the hydrophilic compound, greater than 4 repeats of the hydrophilic compound, greater than 5 repeats of the hydrophilic compound, greater than 6 repeats of the hydrophilic compound, greater than 7 repeats of the hydrophilic compound, or greater than 8 repeats of the hydrophilic compound.

In some embodiments, the hydrophilic spacer includes 1 to 10 hydrophilic blocks, with each block including 1 to 15 repeats of the hydrophilic compound. In some embodiments, the hydrophilic spacer includes 1 to 10 hydrophilic blocks, with each block including 1 to 10 repeats of the hydrophilic compound. In some embodiments, the hydrophilic spacer includes 1 to 6 hydrophilic blocks, with each block including 2 to 10 repeats of the hydrophilic compound. In some embodiments, the hydrophilic spacer includes 2 to 6 hydrophilic blocks, with each block including 3 to 8 repeats of the hydrophilic compound.

The hydrophilic compound can be included in different variations as part of the hydrophilic block. For example, the hydrophilic spacer can include 1 block including 150 repeats of the hydrophilic compound, 2 blocks each including 50 repeats of the hydrophilic compound, 5 blocks each including 6 repeats of the hydrophilic compound, 2 blocks—one block including 5 repeats of the hydrophilic compound and the other block including 10 repeats of the hydrophilic compound, or any combination of blocks and repeats as disclosed herein.

In some embodiments, the hydrophilic spacer includes a plurality of ethylene glycol repeats. For example, the hydrophilic spacer can include 1 to 150 ethylene glycol repeats, 1 to 120 ethylene glycol repeats, 1 to 100 ethylene glycol repeats, 1 to 90 ethylene glycol repeats, 1 to 80 ethylene glycol repeats, 1 to 70 ethylene glycol repeats, 1 to 60 ethylene glycol repeats, 1 to 50 ethylene glycol repeats, 1 to 40 ethylene glycol repeats, 1 to 30 ethylene glycol repeats, 2 to 150 ethylene glycol repeats, 3 to 150 ethylene glycol repeats, 4 to 150 ethylene glycol repeats, 5 to 150 ethylene glycol repeats, 6 to 150 ethylene glycol repeats, 7 to 150 ethylene glycol repeats, 8 to 150 ethylene glycol repeats, 9 to 150 ethylene glycol repeats, 10 to 150 ethylene glycol repeats, 10 to 140 ethylene glycol repeats, 10 to 130 ethylene glycol repeats, 10 to 120 ethylene glycol repeats, 10 to 110 ethylene glycol repeats, 10 to 100 ethylene glycol repeats, 10 to 90 ethylene glycol repeats, 10 to 80 ethylene glycol repeats, 10 to 70 ethylene glycol repeats, 10 to 60 ethylene glycol repeats, 6 to 60 ethylene glycol repeats, 6 to 40 ethylene glycol repeats, 8 to 35 ethylene glycol repeats, or 10 to 32 ethylene glycol repeats. The ethylene glycol repeats can be included as a hydrophilic block(s) in different variations as described above.

In some embodiments, the hydrophilic spacer includes 1 to 10 hexaethylene glycol blocks (e.g., blocks of six ethylene glycol repeats). In some embodiments, the hydrophilic spacer includes 1 to 5 hexaethylene glycol blocks. In some embodiments, the hexaethylene glycol blocks are directly attached to each other and/or the branching molecule. In some embodiments, the hexaethylene glycol blocks are attached to each other and/or the branching molecule through phosphorothioate bonds, phosphodiester, or cleavable linker (e.g., deoxythymidine (dT), pH-cleavable bond such as ketal).

In some embodiments, the length of the hydrophilic spacer may be adjusted to provide desired properties. For example, ethylene glycol spacers with 18 ethylene glycol repeats can provide a higher binding to albumin. Alternatively, shorter spacers can yield conjugates with increased hydrophobicity, which can result in more tendency to bind lipoprotein complexes in the blood.

In some embodiments, the conjugate includes a lipophilic ligand capable of binding albumin, the lipophilic ligand including two independent lipids, each lipid including a C₁₈ hydrocarbon chain; and a linker attaching the siRNA to the lipophilic ligand, the linker including a branching molecule attached to the siRNA and including at least one branch point having at least two independent branches, and a hydrophilic spacer attaching an individual branch to an individual lipid, the hydrophilic spacer including 1 to 6 hydrophilic blocks, each hydrophilic block including 2 to 10 repeats of ethylene glycol.

### iv. Synthesis of Conjugates

Also provided herein are methods of synthesizing the conjugates. In some embodiments, the method includes solid phase synthesis where the full molecule is made/grown from a solid support, as opposed to solution phase conjugation of the siRNA to the linkers/lipidic moieties post-solid phase synthesis. For example, the branching molecule can be integrated during the solid phase synthesis. The integration of the branching molecule can convert the linear growth to divalent growth (or any number of valency), where the hydrophilic spacers can be added to the two growing chains following the branch point.

Further discussion on the disclosed conjugates can be found in PCT/US2022/042445, which is incorporated by reference herein in its entirety.

### B. Administration

While siRNA-based therapeutics can be limited by rapid renal clearance, nuclease degradation, and inability to target/penetrate cells of interest, the conjugates disclosed herein can provide improved circulation half-life, can shield the siRNA from nucleases, and/or can provide extrahepatic delivery of the siRNAs. In addition, these advantages can be done without an associated carrier composition, such as a polymer or lipid formulation. Accordingly, in some embodiments the conjugate or pharmaceutical composition thereof is administered without an associated carrier composition.

The conjugate can be administered optionally in combination with a pharmaceutically acceptable excipient. Embodiments that administer the conjugate and a pharmaceutically acceptable excipient in combination can also be referred to as a pharmaceutical composition. Examples of pharmaceutically acceptable excipients include, but are not limited to, buffering agents (e.g., phosphate buffered saline, artificial cerebrospinal fluid (aCSF), etc.), carbohydrates (e.g., glucose, trehalose, starch, etc.) solubilizers, solvents, antimicrobial preservatives, antioxidants, suspension agents, penetration/absorption enhancers (e.g., DMSO, ethanol, pyrrolidones, and/or ionic liquids) or a combination thereof. In some embodiments, the pharmaceutically acceptable excipient includes saline, phosphate buffered saline (PBS), albumin, dimethyl sulfoxide, trehalose, sucrose, polyethylene glycol (PEG), an absorption enhancer, or a combination thereof. In some embodiments, the conjugate or pharmaceutical composition thereof does not include a carrier composition, such as a polymer- or lipid-based formulation. In some embodiments, the conjugate is administered in combination with a pharmaceutically acceptable excipient.

The conjugate or pharmaceutical composition thereof can be administered prophylactically or therapeutically. In prophylactic administration, the conjugate can be administered in an amount sufficient to induce a response. In therapeutic applications, the conjugate can be administered to a subject in need thereof in an amount sufficient to elicit a therapeutic effect. An amount adequate to accomplish this is defined as “therapeutically effective dose.” Amounts effective for this use will depend on, e.g., the particular composition of the conjugate regimen administered, the manner of administration, the stage and severity of the disease, the general state of health of the patient, and the judgment of the prescribing physician.

The conjugate or pharmaceutical composition thereof can be delivered via a variety of routes to the subject. The conjugate or pharmaceutical composition thereof can be delivered via parenteral administration or by oral administration. Example delivery routes include, but are not limited to, intradermal, intramuscular, intravenous, subcutaneous, oral, intranasal, intravaginal, transdermal, intraarterial, intratumoral, intrathecal, intracerebroventricular, intraperitoneal, epidermal routes, locally at the site of injury (e.g., site associated with inflammation), inhalation, and intraarticular. In some embodiments, the conjugate or pharmaceutical composition thereof is administered intravenously, subcutaneously, intraarticularly, orally, by inhalation, or locally at a site of inflammation associated with the inflammatory disease. In some embodiments, the conjugate or pharmaceutical composition thereof is administered intravenously, intraarticularly, or locally at a site of inflammation associated with the inflammatory disease. In some embodiments, the conjugate or the pharmaceutical composition thereof is administered intravenously or intraarticularly. In some embodiments, the conjugate or the pharmaceutical composition thereof is administered intravenously. Given that albumin is a serum protein, the conjugate or pharmaceutical composition thereof can be administered intravenously. Following administration, the conjugate can bind albumin. In some embodiments, the conjugate is pre-complexed with albumin prior to administration.

The conjugates and pharmaceutical compositions thereof can be administered at varying suitable dosages, which can depend on a number of different factors, such as state of the inflammatory disease, progression of the inflammatory disease, age of the subject, route of administration, and other factors that would be recognized by the skilled artisan. In general, however, a suitable dose will often be in the range of about 0.01 mg/kg to about 1000 mg/kg, such as about 0.1 mg/kg to about 100 mg/kg, about 0.5 mg/kg to about 500 mg/kg, about 1 mg/kg to about 200 mg/kg, about 1 mg/kg to about 100 mg/kg, about 0.1 mg/kg to about 50 mg/kg, about 1 mg/kg to about 50 mg/kg, or about 0.1 mg/kg to about 60 mg/kg. Useful dosages of the conjugate can be determined by comparing their in vitro activity and in vivo activity in animal models thereof. Methods for the extrapolation of effective dosages in rodents, pigs, and other animals, to humans are known in the art; for example, see U.S. Pat. No. 4,938,949, which is incorporated by reference herein in its entirety.

The conjugate or pharmaceutical composition thereof can be administered at varying times and frequency. For example, the conjugate or pharmaceutical composition thereof can be administered at least once (e.g., as a single dose) over 50 days, such as 40 days, 30 days, 25 days, 20 days, 15 days, 10 days, 5 days, or 1 day. In addition, the conjugate or pharmaceutical composition thereof can be administered as multiple doses over a period of time. For example, the conjugate or pharmaceutical composition thereof can be administered 1×, 2×, 3×, 4×, 5×, or more over 50 days—or a period of time listed above. And, the conjugate or pharmaceutical composition thereof may be administered as a single dose or as divided doses administered at appropriate intervals, for example, as two, three, four or more sub-doses per day. The sub-dose itself may be further divided, e.g., into a number of discrete loosely spaced administrations.

Suitable in vivo dosage to be administered and the particular mode of administration will vary depending upon the age, weight, the severity of the affliction, and subjects treated, the particular compounds employed, and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies.

Dosage amount and interval may be adjusted individually to provide plasma levels of the biologically active agent which are sufficient to maintain the modulating effects, or minimal effective concentration (MEC). The MEC will vary for each agent but can be estimated from in vivo and/or in vitro data. Dosages necessary to achieve the MEC will depend on individual characteristics and route of administration. However, assays well known to those in the art can be used to determine plasma concentrations. Dosage intervals can also be determined using MEC value. Conjugates or pharmaceutical compositions thereof can be administered using a regimen which maintains plasma levels above the MEC for 10-90% of the time, such as between 30-90% or between 50-90%. In cases of local administration or selective uptake, the effective local concentration of the conjugate may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be used in veterinary medicine.

The conjugates or pharmaceutical compositions thereof described herein may be administered with additional compositions to prolong stability, delivery, and/or activity of the conjugate or pharmaceutical composition thereof, or combined with additional therapeutic agents, or provided before or after the administration of additional therapeutic agents. Combination therapy includes administration of a single pharmaceutical dosage formulation containing one or more of the conjugates or pharmaceutical compositions thereof described herein and one or more additional pharmaceutical agents, as well as administration of the conjugate or pharmaceutical composition thereof and each additional pharmaceutical agent, in its own separate pharmaceutical dosage formulation. For example, the conjugates and pharmaceutical compositions thereof as described herein may be administered to a subject with an anti-inflammatory drug.

The disclosed invention has multiple aspects, illustrated by the following non-limiting examples.

### 3. EXAMPLES

### Example 1

### Materials & Methods for Examples 1-8

Reagents. 2′-O-Me and 2′-F phosphoramidites, universal synthesis columns (MM1-2500-1), and all ancillary RNA synthesis reagents were purchased from Bioautomation. Symmetrical branching CED phosphoramidite was obtained from ChemGenes (CLP-5215). Cyanine 5 phosphoramidite (10-5915), stearyl phosphoramidite (10-1979), biotin TEG phosphoramidite (10-1955), hexaethyleneglycol phosphoramidite (10-1918), TEG cholesterol phosphoramidite (10-1976), 5′-Amino-Modifier 5 (10-1905), and desalting columns (60-5010) were all purchased from Glen Research. All other reagents were purchased from Sigma-Aldrich unless otherwise specified.

Conjugate Synthesis, Purification, and Validation. Oligonucleotides were synthesized using modified (2′-F and 2′-O-Me) phosphoramidites with standard protecting groups on a MerMade 12 Oligonucleotide Synthesizer (Bioautomation). Amidites were dissolved at 0.1M in anhydrous acetonitrile with the exception of 2′OMe U-CE phosphoramidite, which utilized 20% anhydrous dimethylformamide by volume as a cosolvent, and stearyl phosphoramidite, which was dissolved in 3:1 dichloromethane:acetonitrile by volume. Coupling was performed under standard conditions, and strands were grown on controlled pore glass with a universal terminus (1 μmol scale, 1000 Å pore size).

Strands were cleaved and deprotected using 1:1 methylamine:40% ammonium hydroxide at room temperature for 2 hours. Lipophilic RNAs were purified by reversed-phase high performance liquid chromatography using a Clarity Oligo-RP column (Phenomenex) under a linear gradient from 85% mobile phase A (50 mM triethylammonium acetate in water) to 100% mobile phase B (methanol) or 95% mobile phase A to 100% mobile phase B (acetonitrile). Oligonucleotide containing fractions were then dried using a Savant SpeedVac SPD 120 Vacuum Concentrator (ThermoFisher). Conjugates were then resuspended in nuclease free water and sterile filtered before lyophilization.

Conjugate molecular weight and purity was confirmed using Liquid Chromatography-Mass Spectrometry (LC-MS) analysis on a ThermoFisher LTQ Orbitrap XL Linear Ion Trap Mass Spectrometer. Chromatography was performed using a Waters XBridge Oligonucleotide BEH C18 Column under a linear gradient from 85% A (16.3 mM triethylamine-400 mM hexafluoroisopropanol) to 100% B (methanol) at 45° C. Control conjugate, si-EG₄₅L₂, molecular weight was validated using MALDI-TOF mass spectrometry using 50 mg/mL 3-hydroxypicolinic acid in 50% water, 50% acetonitrile with 5 mg/mL ammonium citrate as a matrix.

Synthesis of amine-reactive lipids and subsequent modification of oligonucleotides was adapted from methods reported by Prakash, T. P. et al. Fatty acid conjugation enhances potency of antisense oligonucleotides in muscle. Nucleic Acids Res 47, 6029-6044 (2019), which is incorporated herein by reference in its entirety. Briefly, amine-terminated oligonucleotides were speed vacuumed to dryness and desalted to remove MMT groups. Oligonucleotides were then lyophilized followed by reconstitution in 0.1 sodium tetraborate (pH 8.5) to a concentration of 500 uM. PFP-modified lipid was dissolved into a mixture of acetonitrile, DMSO, and triethylamine (70:29:1 by volume) at a concentration of 7 uM. Aqueous oligonucleotide was added dropwise to the organic solution for a 1:40 molar ratio of oligonucleotide-amine:amine-reactive lipid (approximately 25% 0.1M sodium tetraborate, 75% organic mixture). Solution was stirred overnight and desalted prior to purification and characterized.

Purified oligonucleotide was resuspended in 0.9% sterile saline and annealed to its complementary strand by heating to 95° C. and cooling stepwise by 15° C. every 9 min until 25° C. on a T100 Thermal Cycler (BioRad).

Duplexes directly bound to albumin were synthesized in a two-step, one-pot reaction. Briefly, conjugate covalently bound to albumin was synthesized by first reacting azido-PEG3-maleimide (Click Chemistry Tools) with the free thiols on human (1 free SH) or mouse (2 free SH). Albumin was dissolved in PBS with 0.5M EDTA to a final concentration of 10 mM. Anhydrous DMF was used to solubilize and activate azido-PEG₃-maleimide. DBCO-modified siRNA duplex in PBS was reacted at a 1:1 ratio of DBCO groups:free SH groups and allowed to incubate at room temperature for 4 hours. To remove any siRNA that did not react with albumin, or reacted only with the azido linker, the resulting solution underwent 10 rounds of centrifugation in a 30 kDa cutoff Amicon filter at 14,000×g for 10 minutes for each round. Conjugation was confirmed by gel electrophoresis of precusor DBCO-siRNA alongside resulting siRNA-DBCO-albumin.

Cell Culture. Cells were cultured in Dulbecco's modified eagle's medium (DMEM, Gibco), containing 4.5 g/L glucose, 10% FBS (Gibco), and 50 μg/mL gentamicin. All cells were tested for Mycoplasma contamination MycoAlert Myocplasma Detection Kit (Lonza).

In Vitro Knockdown Experiments. For lipofection-mediated knockdown experiments, luciferase-expressing MDA-MB-231s were seeded at 4,000 cells per well in 96 well plates in complete media. After 24 h, cells were treated with siRNA (25 nM) using Lipofectamine 2000 (ThermoFisher) in OptiMEM according to manufacturer protocol, replacing with complete media at 24 h post-transfection, and measuring luciferase activity at 48 h post-transfection in cells treated for 5 min with 150 μg/mL D-Luciferin, potassium salt (ThermoFisher) using an IVIS Lumina III imaging system (Caliper Life Sciences).

Serum Stability. siRNA (0.1 nmol) in 60% fetal bovine serum in PBS was incubated at 37° for 0-48 h, then assessed on a 2% agarose gel in 1×TAE Buffer. Gels were stained with GelRed Nucleic Acid Stain (Biotium) according to the manufacturer's protocol.

Biolayer Interferometry. Binding kinetics were measured by biolayer interferometry using an Octet RED 96 system (ForteBio). Duplexes synthesized with TEG-Biotin on the 5′ terminus of the antisense strand were diluted to 500 nM in Dulbecco's phosphate buffered saline containing calcium and magnesium (DPBS^{+/+}) and loaded on a Streptavidin Dip and Read Biosensor (ForteBio) for 600 sec. Baseline was then established over 120 sec in DPBS^{+/+} followed by association to either human or mouse serum albumin in DPBS^{+/+} over 300 sec. Subsequently, the biosensor was immersed in DPBS^{+/+} for 300 sec to measure dissociation. All steps were conducted at 30° C. and 1000 rpm. The binding values were measured using Octet Data Analysis HT Software. Reference biosensor values (biotinylated conjugate bound with no analyte) were subtracted to account for signal background. Y axes were aligned to the average of the baseline step. Interstep correction was performed by aligning to the dissociation step, and noise filtering was performed. Global analysis was performed to derive constants simultaneously from all tested analyte concentrations.

Critical Micelle Concentration. A serial dilution of duplexes was prepared in a 96-well plate from 20 μM to 10 nM in 50 uL of DPBS (Ca²⁺/Mg²⁺ free). Nile Red (1 μL of a 0.5 mg/mL stock solution) was added to each well. Samples were then incubated in the dark with agitation at 37° C. for 2 h, and fluorescent intensity was measured on a plate reader (Tecan) at excitation 535±10 nm and emission 612±10 nm. The critical micelle concentration was defined as the intersection point on the plot of the two linear regions of the Nile red fluorescence versus the duplex concentration.

Gel Migration Shift Experiments. Binding of siRNA conjugates (0.1 nmol) to human or mouse serum albumin (in 5×molar excess) incubated for 30 min at 37° C. was assessed by migration through 4%-20% polyacrylamide gels (Mini-Protean TGX). siRNA was visualized with GelRed Nucleic Acid Stain (Biotium) for ultraviolet imaging, and proteins were visualized with Coomassie blue and visible light imaging.

Conjugation efficacy of DBCO-modified siRNA duplex with azide-modified albumin was visualized using the Agilent Protein 230 Assay on the Agilent 2100 Bioanalyzer according to manufacturer instructions.

Long-Term Fluorescence of Blood Samples. Longer term pharmacokinetic profiles of siRNA conjugates were established by measuring fluorescence of blood samples taken at various time points from 5 min to 24h. Blood was sampled in the contralateral vein from injection (˜10 μL) using EDTA coated capillary tubes and stored at −80° C. Blood was then thawed and diluted 40× with phosphate buffered saline in a 96-well plate and fluorescent intensity was measured.

Intravital Microscopy and Biodistribution. Microscopy was performed using a Nikon Czsi+ system. Isoflurane-anesthetized, 6-8 week old male CD-1 mice (Charles River) were immobilized on a heated confocal microscope stage for ear vein imaging. Mouse ears were depilated and then immobilized on a glass coverslip using microscope immersion fluid. Ear veins were detected using light microscopy, and images were focused to the plane of greatest vessel width, where flowing red blood cells were clearly visible. Once in focus, confocal laser microscopy was used to acquire one image per second, at which point Cy5-labeled siRNA (1 mg/kg) in 100 μL was delivered via tail vein. Fluorescent intensity within a circular region of interest (ROI), drawn in the focused vein, was used to measure fluorescence decay. Values are normalized to maximum initial fluorescence and fit to a one-compartment model in PK Solver to determine pharmacokinetic parameters.

Approximately 45 min after delivery of Cy5-labeled siRNA, blood was collected by cardiac puncture using EDTA-coated tubes and used for plasma isolation. Cy5 fluorescence was quantified in heart, lung, liver, kidney, and spleen using IVIS Lumina Imaging system (Xenogen Corporation) at excitation and emission wavelengths of 620 and 670 nm, respectively, using Living Image software version 4.4.

Size Exclusion Chromatography (SEC). Murine plasma was filtered (0.22 μm) then injected into an AKTA Pure Chromatography System (Cytiva) with three inline Superdex 200 Increase columns (10/300 GL) for fractionation at 0.3 mL/min using Tris running buffer (10 mM Tris-HCl, 0.15M NaCl, 0.2% NaN₃) into 1.5 mL fractions with a F9-C 96-well plate fraction collector (Cytiva). Cy5 fluorescence was measured in fractions (100 μL) in black, clear-bottom, 96-well plates (Greiner-Bio-one REF 675096) on a SynergyMx (Biotek) at a gain of 120, excitation 642/9.0, emission 675/9.0. Fraction albumin-bound conjugate was determined by taking the sum of fluorescence intensity for fractions associated with albumin elution divided by the sum of fluorescence intensity for all fractions collected. Albumin-associated fractions were determined by running known protein standards through the SEC system and examining A280 of eluent from each of the fractions.

Statistical Analyses. Data were analyzed using GraphPad Prism 7 software (Graphpad Software, Inc.) Statistical tests used for each data are provided in the corresponding figure captions. For all figures, *p≤0.05 **p≤0.01 ***p≤0.001 ****p≤0.0001. All plots show mean±standard deviation.

### Example 2

### Divalent Lipid Modifier Improves Bioavailability of Chemically Stabilized siRNAs

To finely tune and examine the structure-function relationship of siRNA variants, a library of siRNA-lipid conjugates was generated using solid phase synthesis, which maximizes product yield, purity, and reproducibility compared to previously reported two-step solution phase conjugation as described in Sarett, S. M. et al. Lipophilic siRNA targets albumin in situ and promotes bioavailability, tumor penetration, and carrier-free gene silencing. Proceedings of the National Academy of Sciences of the United States of America 114, E6490-E6497 (2017), which is incorporated herein by reference in its entirety. The synthesized siRNAs were designed to be fully stabilized with alternating 2′F and 2′OMe modifications in a “zipper” pattern and terminal phosphorothioate linkages. These modifications can confer endonuclease and exonuclease resistance. It was demonstrated that these stabilizing siRNA modifications maintain gene silencing potency and provide serum stability, while traditional Dicer substrate siRNAs are similarly potent but degrade within 4 h of serum challenge.

Valency may affect bioavailability and pharmacodynamics of lipid end-modified siRNA conjugates in vivo. Conjugation to one or two 18-carbon stearyls was focused on, an albumin-binding lipid with higher albumin affinity than those with shorter lipid chain lengths, for initial assessment of modifier valency on siRNA pharmacokinetics. Absolute circulation half-life (t_{1/2}) was measured using real-time fluorescence imaging of Cy5-labeled siRNA conjugates within mouse vasculature, revealing increased t_{1/2} (46±5.9 min) of siRNA conjugated to divalent (L₂) over monovalent (L₁) stearyl (28±4.2 min). Importantly, L₂-conjugated siRNA (siRNA-L₂) showed diminished kidney accumulation compared to siRNA-L₁, suggesting that renal clearance, the primary elimination mechanism of circulating siRNAs, is reduced with siRNA-L₂. The remainder of the studies, therefore, focused on a divalent lipid design.

### Example 3

### Hydrophilic Linker Length Increases In Vitro Albumin Affinity and Reduces Self-Assembly of Lipid-siRNA Conjugates

Based on the improved performance of siRNA-L₂ over siRNA-L₁ in vivo, siRNA-L₂ was modified to assess the functional effects of structural modification of the hydrophilic linker between the lipid and siRNAs. Specifically, the number of ethylene glycol (EG) repeats were progressively increased from no EG repeats [si<(EG₀L)₂] to 30 EG repeats [si<(EG₃₀L)₂]; the EG repeats were added in increments of 6, using a hexaethylene phosphoramidite. Two previously described serum protein-binding siRNA conjugates, cholesterol-TEG-siRNA (si-chol) and si-EG₄₅<L₂, were synthesized as comparative references.

Each siRNA<(EG×L)₂ was incubated with human serum albumin to assess albumin-siRNA complex formation by electrophoretic mobility shift assay. These studies revealed that, while electrophoretic mobility of free siRNA was unaffected by albumin, the mobility si<(EG₀L)₂, si<(EG₆L)₂, si<(EG₁₈L)₂, and si<(EG₃₀L)₂ was lowered upon exposure to albumin, consistent with the high molecular weight of the complex formed by albumin and siRNA conjugates. Similarly, si-cholesterol, and si-EG₄₅<L₂ also displayed albumin-dependent mobility shifts in this assay. However, super-shifting of si-EG₄₅<L₂ was seen in both the presence and absence of albumin, suggesting that si-EG₄₅<L₂ may harbor some self-association properties which were not seen in si<(EG×L)₂ conjugates. Similar results were observed using mouse serum albumin.

Albumin association and dissociation kinetics of the si<(EG×L)₂ variants were studied further using biolayer interferometry. Free siRNA did not exhibit binding with albumin, while si-cholesterol exhibited moderate albumin binding. Although si<(EG₀L)₂ displayed decreased HSA binding response compared to si-cholesterol, siRNA conjugates harboring a greater number of EG spacers within the linker element had progressively higher affinity for HSA, with both si<(EG₁₈L)₂ (K_{D}=30±0.3 nM) and si<(EG₃₀L)₂ (K_{D}=9.49±0.1 nM) exhibiting higher affinity albumin binding than si-cholesterol and si-EG₄₅<L₂. The substantial difference in albumin binding response between variants underscores the role of the EG repeats within the linker region.

Amphiphilic lipid-modified nucleic acids have a tendency to self-assemble into micellar structures, particularly when using long lipid chains. It is possible that self-aggregation of amphiphilic siRNA-lipid conjugates is a competing interaction that might interfere with albumin association, particularly if lipid tails can become sequestered in the core of a self-assembled structure, where they would be rendered unavailable for interaction with the fatty acid binding pockets of albumin. Thus, the critical micelle concentration (CMC) was determined for each si<(EG×L)₂ to establish the impact of linker length on siRNA conjugate self-assembly. The si-cholesterol exhibited a relatively high CMC (3430±350 nM), suggesting a low tendency for si-cholesterol to self-associate. This is consistent with the bulky structure of cholesterol, which is not amenable to close packing like lamellar long-chain lipids. In contrast, si-EG₄₅<L₂ exhibited a lower CMC (1860±60 nM), indicating a higher tendency towards self-association. Interestingly, si<(EG₀L)₂, which lacks any EG spacer in the linker element, exhibited the lowest CMC (1040±23 nM) and thus the highest propensity for self-association, while the increased number of EG repeats in si<(EG₁₈L)₂ and si<(EG₃₀L)₂ correlated with the highest CMCs (3260±190 nM and 3330±210 nM), and thus the lowest tendency towards self-association.

### Example 4

### Hydrophilic Linker Length Influences Pharmacokinetics and In Vivo Plasma Disposition of Lipid-siRNA Conjugates

The structure of hydrophobic modifications on siRNA can be tuned to direct siRNA binding to different serum components, such as lipoproteins and albumin, after intravenous administration, which can consequently modify pharmacokinetics and biodistribution. The in vivo half-life of human albumin is approximately 19 days, making it a good candidate for improving the pharmacokinetics of candidate therapeutics.

Each of the siRNA conjugates was intravenously administered to mice to understand how EG repeats within the linker affect conjugate pharmacokinetics. Intravital fluorescence microscopy of Cy5-labeled siRNA conjugates flowing through vessels of the mouse ear demonstrated the rapid and complete diminution of circulating free siRNA within the first 30 min post-treatment, while serum component-binding si-cholesterol and si-EG₄₅<L₂ retained some observable circulating siRNA. Interestingly and unexpectedly, increased EG repeats correlated with increased retention of circulating siRNA in si<(EG×L)₂ conjugates to a point, but once the linker became too long (e.g., si<(EG₃₀L)₂), retention was reduced, with si<(EG₁₈L)₂ showing maximal circulation retention of the variants tested. Real-time collection of vascular fluorescence imaging data throughout the first hour post-treatment enabled calculation of absolute half-life (t_{1/2 abs}), demonstrating the substantially prolonged t_{1/2 abs} of si<(EG₁₈L)₂, which was greater than what was observed for si<(EG₃₀L)₂, and nearly 5 times that of si<(EG₀L)₂ (Table 1). Approximately 45 min after injection, there was significantly more renal accumulation, the primary clearance path for oligonucleotide-based therapeutics, of the parent siRNA and control conjugate, si-EG₄₅<L₂.

TABLE 1
Pharmacokinetic parameters for siRNA conjugate
library determined from intravital microscopy.

| | | t_{1/2} | AUC₀₋₃₅ₘ | CL |
|---|---|---|---|---|
| | | (min) | (μg · min)/(mL) | (mL)/(min) |
| EG | siRNA | 14 ± 3.5 | 210 ± 25 | 0.069 ± |
| Variants | | | | 0.012 |
| | si-EG₃-cholesterol | 28 ± 12 | 410 ± 42 | 0.025 ± |
| | | | | 0.0079 |
| | si-EG₄₅ < L₂ | 27 ± 5.3 | 390 ± 25 | 0.026 ± |
| | | | | 0.0046 |
| | si < (EG₀L)₂ | 33 ± 6.6 | 416 ± 55 | 0.028 ± |
| | | | | 0.0059 |
| | si < (EG₆L)₂ | 36 ± 4.6 | 450 ± 15 | 0.019 ± |
| | | | | 0.0022 |
| | si < (EG₁₈L)₂ | 64 ± 23 | 470 ± 52 | 0.012 ± |
| | | | | 0.0032 |
| | si < (EG₃₀L)₂ | 37 ± 6.6 | 440 ± 36 | 0.019 ± |
| | | | | 0.0043 |
| PS | si < (EG₁₈L)₂No 5′Se | 37 ± 12 | 430 ± 50 | 0.020 ± |
| Variants | PS | | | 0.0060 |
| | si < (EG₁₈L)₂No 5′Se | 15 ± 1.5 | 300 ± 36 | 0.047 ± |
| | or Binder PS | | | 0.0070 |
| Branching | si-(EG₁₈) < L₂ | 67 ± 32 | 490 ± 35 | 0.012 ± |
| Variants | | | | 0.0070 |
| | si-(EG₃₆) < L₂ | 41 ± 13 | 450 ± 27 | 0.018 ± |
| | | | | 0.0050 |
| Lipid | si < (EG₁₈L_{diacid})₂ | 47 ± 21 | 469 ± 64 | 0.017 ± |
| Variants | | | | 0.0073 |
| | si < (EG₁₈L_{unsaturated})₂ | 34 ± 26 | 391 ± 121 | 0.029 ± |
| | | | | 0.016 |

It is hypothesized, without being bound by a particular theory, that the diminished renal clearance of the si<(EG×L)₂ variants over si-EG₄₅<L₂ is due to the presence of hydrolytically degradable ester bonds located in the structure of DSPE-PEG₂₀₀₀ used to make si-EG₄₅<L₂. Albumin itself is known to possess intrinsic esterase activity, making the hydrolytic stability of drugs that interact with it particularly important. Hydrolysis of this ester would be expected to release the siRNA from albumin, possibly prematurely in the circulation, resulting in renal clearance and shorter circulation time that is more analogous to the non-modified parent siRNA structure. Conjugates that remain albumin-bound, by comparison, can evade renal clearance through albumin's natural reabsorption in the renal proximal tubule where, after endocytosis by the megalin-cubilin complex, the neonatal Fc receptor redirects albumin and its associated cargo back to the interstitial space, facilitating its return to the circulation via the lymphatics.

Plasma was collected from mice treated intravenously with Cy5-labeled siRNA conjugates and analyzed by size exclusion chromatography to measure the level of each candidate's association with albumin versus other plasma fractions (e.g., lipoproteins). Cy5 fluorescence was detected in albumin-containing plasma fractions, as well as in fractions not representative of albumin elution. Notably, plasma isolated from mice treated with si<(EG₁₈L)₂, the most long-circulating on the investigated conjugates, exhibited the most robust peak within the albumin-containing fraction at approximately 75% bound. This observation is consistent with the BLI data showing the high affinity of si<(EG₁₈L)₂ with albumin and also suggests a positive correlation between the percent of conjugate that is albumin-bound in vivo and the circulation half-life.

It is interesting and unexpected that in vitro albumin binding affinity analyses indicate that si<(EG₃₀L)₂ binds albumin more favorably than si<(EG₁₈L)₂, while albumin association in vivo is greater with si<(EG₁₈L)₂. This may be attributable to the larger entropic penalty of binding incurred for this larger and more flexible molecule that is mitigated by the ideal conditions of in vitro testing but becomes more apparent in vivo.

### Example 5

### Phosphorothioate Linkages of Lipid-Modified Terminus Improves Conjugate Performance In Vitro and In Vivo

Deeper structural interrogation of si<(EG₁₈L)₂ was next focused on by assessing the impact of the phosphorothioate (PS) bonds at the 5′ sense terminus and between the EG₆ repeating units of the linker. Terminus stabilization with PS linkages in lieu of phosphodiester (PO) linkages has significant effects on performance of siRNA-based therapeutics by conferring exonuclease resistance and can help enable extrahepatic, carrier-free gene silencing applications. Variants of si<(EG₁₈L)₂ with PS bonds removed from the 5′ sense (Se) terminus (si<(EG₁₈L)₂ No 5′Se PS) or removed from both the 5′ sense terminus and each of the bonds in the linker to the stearyl groups (si<(EG₁₈L)₂ No 5′Se or Binder PS) were synthesized and studied using biolayer interferometry to determine albumin binding kinetics. Both variants exhibited comparable albumin affinity as the parent construct with K_{D} values only varying ±2 nM. However, removing the PS bonds from the linker significantly increased the critical micelle concentration compared to just removing it from the 5′ sense terminus (2755±526 to 3798±225 nM), suggesting a lower tendency to self-assemble without the more hydrophobic PS bonds located on the linker.

Real-time, intravital microscopy of fluorescently labeled conjugates in circulation was performed to determine the effect of PS content on circulation time. Removal of PS bonds from both the 5′ sense terminus and the 5′ sense terminus/binder resulted in significantly diminished pharmacokinetic profiles compared to parent construct si<(EG₁₈L)₂ with circulation half-lives reduced to 37±12 min and 15±1.5 min respectively from 64±23 min. Biodistribution of conjugates approximately 45 min after treatment demonstrated significant differences in PS-dependent accumulation in both the lungs and the liver. Plasma collected from mice treated intravenously with each siRNA conjugate to examine proteins bound in vivo demonstrated 10-fold lower relative albumin binding by si<(EG₁₈L)₂ siRNA conjugate when the PS bonds were not used at the 5′ sense terminus. These combined data suggest that the PS bonds within the linker facilitate albumin binding. The diminished pharmacokinetics observed with the removal of phosphorothioate bonds is most likely due to reduced albumin association and not due to degradation. This conclusion is supported by the observation that, unlike ester-containing si-EG₄₅<L₂. the kidney accumulation of the conjugate without PS bonds was similar to the fully modified parent construct.

### Example 6

### Position of Branching Point in Divalent Lipid Conjugate affects Conjugate Performance In Vitro and In Vivo

It was sought to determine whether the position of the branching point in the divalent si<(EG₁₈L)₂ conjugate influences candidate function. This study was motivated by the desire to further confirm the observation that albumin association is driven by reduced tendency to self-assemble rather than simply the relative level of conjugate hydrophilic linker content or linker length. It is hypothesized, without being bound by a particular theory, that placement of the branching point distal to the repeating EG linker and immediately proximal to the stearyl groups increases apparent hydrophobicity and consequent self-assembly by constraining the stearyl groups to remain tightly packed together. Since the greatest siRNA circulation time was seen with the si<(EG₁₈L)₂ conjugate, which correlated with increased albumin binding in vivo, two additional iterations of si<(EG₁₈L)₂ were generated with a distal branching location, one matched for overall ethylene glycol content (si-EG₃₆<L₂) and one matched for the distance between the siRNA and its lipid tail (si-EG₁₈<L₂). Biolayer interferometry measurement of albumin binding in vitro interestingly showed that both new branching point variants had lower albumin binding affinity relative to the parent construct si<(EG₁₈L)₂. The CMC was measured by Nile Red encapsulation and both si-EG₁₈<L₂ and si-EG₃₆<L₂, similarly to control construct si-EG₄₅<L₂, exhibited significantly lower values (1838±117 and 2293±132 nM) compared to si<(EG₁₈L)₂ (3255±192 nM). These data suggest that the more proximal branch site increases albumin association and consequent siRNA activity at least partially due to decreased tendency of amphiphilic siRNA conjugates with this feature to self-assemble.

Intravital microscopy of circulating fluorescent conjugates showed that there was not a significant difference in absolute circulation half-life among the branching architecture variants. However, biodistribution of the fluorescently labeled conjugates approximately 45 min after administration revealed significantly higher liver accumulation of si-EG₁₈<L₂ and si-EG₃₆<L₂ compared to si<(EG₁₈L)₂. This observation may be explained by a tendency to bind a greater fraction of lipoproteins over albumin in vivo which can preferentially traffic the conjugates to the liver. Indeed, plasma isolated from mice treated with the conjugates and analyzed by SEC for associated proteins demonstrated significantly decreased albumin binding among branching architecture variants si-EG₁₈<L₂ and si-EG₃₆<L₂ (˜5%) compared to si<(EG₁₈L)₂ (˜75%). In sum, these data show that the distal placement of the branching point increases self-assembly and association with lipoproteins at the cost of diminished association with and piggybacking upon serum albumin.

### Example 7

### Hydrophobicity of Lipid is more Important than Binding Affinity for Conjugate Performance

It was next sought to investigate whether the nature of the Cis lipid itself has important implications in conjugate performance. To this end, two variants were synthesized—one with the carboxyl terminal still intact (si<(EG₁₈L_{diacid})₂) and one with a double bond (si<(EG₁₈L_{unsaturated})₂). The carboxyl handle of fatty acids is usually consumed in conjugation reactions. However, the development of GLP-1 agonist drug Semaglutide demonstrated the importance of this moiety for albumin-binding drugs. Indeed, by restoring the carboxyl on their lipid-peptide, they found significant increases in albumin affinity and circulation half-life. Thus, it was sought to explore whether having this group in this system would improve performance.

Further, interest in testing a variant with a double bond was motivated by 1) evidence suggesting that oleate, an unsaturated variant of stearate, possesses a higher affinity for albumin and 2) the idea that double bonds introduce “kinks” into lipid chains that prevent close packing and may therefore deter self-assembly, which was shown to correlate with poorer albumin binding. To test these hypotheses, si<(EG₁₈-Amine)₂ was synthesized and conjugated amine-reactive PFP-modified lipid variants on to the terminus of the conjugates. Both the diacid and unsaturated variants of the conjugate exhibited comparable kidney accumulation and absolute circulation half-life. However, si<(EG₁₈L_{unsaturated})₂ demonstrated significantly diminished albumin binding in vivo compared to its saturated and diacid counterparts (˜40% bound versus ˜75-80% bound). Based on the correlations of albumin-bound in vivo observed previously, only the diacid variant was further characterized. Strikingly, si<(EG₁₈L_{diacid})₂ showed a substantially stronger binding response to albumin, with affinity for human serum albumin approximately two orders of magnitude superior to its non-acid counterpart (si<(EG₁₈L_{diacid})₂ K_{D}=0.15+0.002 nM and si<(EG₁₈L)₂ K_{D}=30±0.3 nM). To determine whether the impact of this increased affinity for albumin would be captured by circulation half-life at longer time points, blood was sampled from mice injected intravenously with 5 mg/kg of either fluorescently labeled si<(EG₁₈L)₂ or its diacid counterpart but found no significant difference in their PK profiles.

### Example 8

### Albumin-Binding Lipophilic siRNA Conjugates Outperform siRNA Directly Conjugated to Albumin

To further interrogate the appeal of an albumin-binding, lipophilic conjugate, an siRNA duplex was synthesized directly, covalently bound to mouse serum albumin. It was sought to determine whether the lipid-mediated, reversible binding was preferable to maximized albumin-bound delivery. These complexes were synthesized by leveraging the two free thiol groups present on mouse serum albumin as a handle for modifying with an azido-PEG₃-maleimide linker followed by reacting with DBCO-modified siRNA duplex. Gel electrophoresis demonstrated an upward shift of resulting product relative to the DBCO-duplex precursor, suggesting successful conjugation and removal of unreacted ligands. Fluorophore-labeled duplex was additionally used to validate that A260 readouts of product agreed with fluorescent readouts for quantification of siRNA in the resulting complex. Plasma isolated from mice injected with the siRNA-MSA complex demonstrated that approximately 80% of the siRNA was associated with fractions associated with albumin. Strikingly, however, the observed half-life of the siRNA covalently bound to albumin was greatly diminished compared to the lipophilic siRNA conjugate. Previous reports have shown that cell surface glycoproteins gp18 and gp30 can bind to covalently modified albumin and act as scavenger receptors that traffic the modified albumin for lysosomal degradation. This is further supported by the organ biodistribution of the siRNA taken from the same mice, which shows no significant difference in kidney levels between the groups. This suggests that liberation from the albumin resulting in renal clearance is not responsible for the reduction in circulation half-life.

The examples disclosed herein show that systematic variation of lipid-siRNA conjugate valency, linker length, phosphorothioate bonds, lipid chemistry, and linker branching architecture impacts albumin binding, pharmacokinetics, and tissue biodistribution. It is shown that lipid valency has a significant impact on pharmacokinetics. Further, the addition of a hydrophilic linker improves albumin binding, but not indiscriminately. The data suggest that there is an advantageous length of hydrophilic linker, e.g., [si<(EG₁₈L)₂], and that this linker improves albumin-binding when placed after the branching point of the divalent moiety. Notably, designs with the hydrophilic linker before the branching point of the divalent structure, when matched for both overall hydrophobicity or length between the siRNA and lipids, showed inferior albumin association in plasma in vivo, which may be attributable to greater lipoprotein association and propensity for self-assembly (lower CMC). The examples also showed that phosphorothioate, rather than phosphodiester, linkages are beneficial both within the linker structure and sense strand terminus where the albumin-binding moiety is located, possibly to promote plasma protein binding. It was additionally demonstrated that the increased albumin affinity conferred by keeping the carboxyl handle of the fatty acid intact is outcompeted by the need for hydrophobicity to achieve silencing efficacy. Further, the examples suggest that siRNA benefits from being reversibly, rather than covalently, bound to albumin.

Overall, this work has important implications for the delivery of siRNAs to extrahepatic targets, a goal that has remained clinically elusive. Albumin is known to accumulate at sites of inflammation and vascular leakiness. Therefore, the insights gleaned from the development of si<(EG₁₈L)₂ as a beneficial siRNA structure for in situ albumin piggybacking can have far reaching implications for the development and improvement of extrahepatic, carrier-free siRNA therapeutics.

### Example 9

### Treatment of Inflammatory Diseases

### Methods

Materials. 2′-O-Me and 2′-F phosphoramidites and universal synthesis columns (MM1-2500-1) were purchased from Bioautomation. Symmetrical branching CED phosphoramidite was obtained from ChemGenes (CLP-5215). Cyanine 5 phosphoramidite (10-5915), stearyl phosphoramidite (10-1979), hexaethyleneglycol phosphoramidite (10-1918), TEG cholesterol phosphoramidite (10-1976), and desalting columns (60-5010) were all purchased from Glen Research. Unless otherwise stated, materials and reagents were purchased from Fisher Scientific (Waltham, MA, USA) or Sigma-Aldrich (St. Louis, MO, USA). The specific MMP13 inhibitor (CL-82198) was purchased from MedChemExpress (HY-100359). The broad-MMP inhibitor Marimastat was purchased from APExBio (A4049). Zilretta and methylprednisolone were purchased as clinical formulations through the Vanderbilt VUMC pharmacy. All drugs were formulated per material data sheet recommendations for in-vivo use. C2C ELISA kit and C1,2C antibody was obtained from IBEX Pharmaceuticals.

siRNA Screening, Modifications, Synthesis, Purification, Validation, Annealing, and Storage. Oligonucleotides were synthesized using modified (2′-F and 2′-O-Me) phosphoramidites with standard protecting groups on a MerMade 12 Oligonucleotide Synthesizer (Bioautomation). Original sequences were purchased from Dharmacon and screened in ATDC5 cells for efficacy before being chemically stabilized in-house. Amidites were dissolved at 0.1M in anhydrous acetonitrile except for 2′OMe U-CE phosphoramidite which utilized 20% anhydrous dimethylformamide by volume as a cosolvent and stearyl phosphoramidite which was dissolved in 3:1 dichloromethane:acetonitrile by volume. Coupling was performed under standard conditions, and strands were grown on controlled pore glass with a universal terminus (1 μmol scale, 1000 Å pore size).

Strands were cleaved and deprotected using 1:1 methylamine:40% ammonium hydroxide at room temperature for 2 hours. Lipophilic RNAs were purified by reversed-phase high performance liquid chromatography using a Clarity Oligo-RP column (Phenomenex) under a linear gradient from 85% mobile phase A (50 mM triethylammonium acetate in water) to 100% mobile phase B (methanol) or 95% mobile phase A to 100% mobile phase B (acetonitrile). Oligonucleotide containing fractions were then dried using a Savant SpeedVac SPD 120 Vacuum Concentrator (ThermoFisher). Conjugates were then resuspended in nuclease free water and sterile filtered before lyophilization.

Conjugate molecular weight and purity was confirmed using Liquid Chromatography-Mass Spectrometry (LC-MS) analysis on a ThermoFisher LTQ Orbitrap XL Linear Ion Trap Mass Spectrometer. Chromatography was performed using a Waters XBridge Oligonucleotide BEH C18 Column under a linear gradient from 85% A (16.3 mM triethylamine-400 mM hexafluoroisopropanol) to 100% B (methanol) at 45° C. Mass and chromatographic characterization was then analyzed.

Purified oligonucleotide was resuspended in 0.9% sterile saline and annealed to its complementary strand by heating to 95° C. and cooling stepwise by 15° C. every 9 minutes until 25° C. on a T100 Thermal Cycler (BioRad). Annealed strands were frozen at their specified concentrations at −80° C. until their use.

Cell Culture and Cell Viability. Immortalized mouse chondrogenic ATDC5 cells procured from ATCC were cultured in DMEM/F-12, GlutaMAX medium with 10% serum and 1% penicillin/streptomycin. Primary guinea pig knee joint chondrocytes were cultured in DMEM with 10% serum and 1% penicillin/streptomycin. Cells were incubated at 37° C. in 5% carbon dioxide. The CellTiter-Glo (Promega Corporation, Madison, Wisconsin, USA) assay was used to assess cytotoxicity in accordance with the manufacturer's standard protocol. All cells for this manuscript tested negative for Mycoplasma contamination by MycoAlert Mycoplasma Detection Kit (Lonza).

Mouse and Guinea Pig MMP13 siRNA Sequence Screening and Modified siRNA Knockdown. Seven candidate siRNA sequences targeting different sites of the murine MMP13 gene were screened in ATDC5 cells stimulated with the inflammatory cytokine murine TNFα (20 ng/mL) for 24 hours. 4 candidate siRNA sequences targeting different sites of the guinea pig MMP13 gene were screened in primary guinea pig knee chondrocytes stimulated with the inflammatory cytokine guinea pig TNFα (20 ng/mL) for 24 hours. 21 base pair-length oligonucleotides (21-mers) used in these studies were purchased from Dharmacon, Incorporated (Lafayette, CO, USA). The lead sequences were then made into a 19-mer by removing the 3′UU's and then formulated into the enhanced stabilization chemistry (ESC) i.e., “zipper” in-house. siRNA was complexed (either 50 nM or 100 nM) using Lipofectamine 2000 (ThermoFisher) according to the manufacturer's protocol in OptiMEM and treatment was performed for 24 hours before being replaced with complete media containing TNFα for another 24 hours before RNA being extracted. For carrier-free studies, siRNAs were diluted in OptiMEM to a 1000 nM dose and placed on cells for 72 hours before RNA being extracted. replacing with complete media at 24 h post-transfection.

RNA harvest, cDNA Synthesis, and qRT-PCR. After animal euthanasia, hindlimbs were cleaned of excess muscle and placed in RNAlater solution (ThermoFisher Scientific; Waltham, MA). Using a dissection microscope, an 11-blade and scalpel, and forceps, the anterior synovial tissue pad was isolated by pulling on the remaining quadriceps tendon and carefully cutting into the joint capsule at the start of the femur bone. Cartilage from tibial and femoral articular surfaces were removed from the bone surface, and menisci, present in between the femur and tibia, was harvested. For RNA extraction cartilage/meniscal tissue was mixed with synovial tissue in equal mass ratios for all samples and labelled as ‘whole joint’ mRNA. RNA was extracted and purified using the RNeasy Plus Mini Kit from Qiagen (Venlo, Netherlands) and quantified using a NanoQuant plate from Tecan in a micro plate reader (Tecan Infinite 500, Tecan Group Ltd., Mannedorf, Switzerland). The RNA was converted to cDNA using the iScript cDNA Synthesis Kit from Bio-Rad (Hercules, California, USA). Gene expression was calculated by the ΔΔCt method, normalizing to glyceraldehyde 3-phosphate dehydrogenase (GAPDH) and beta-actin (ACTB). TaqMan reagents were purchased from Thermofisher Scientific (Waltham, Massachusetts, USA) and used according to provided protocols, using appropriate primers. For all experiments, no reverse transcriptase (No RT), no primers, and no cDNA controls were performed.

| | |
|---|---|
| Murine Taqman Probes: | |
| ACTB: Mm02619580_g1 | GAPDH: Mm99999915_g1 |
| MMP13: Mm00439491_m1 | IL-1β: Mm00434228_m1 |
| IL-6: Mm00446190_m1 | COX2: Mm03294838_g1 |
| TNFa: Mm00443258_m1 | SPARC: Mm00486332_m1 |
| Caveolin-1: Mm00483057_m1 | FcRn: Mm00438887_m1 |
| NGF: Mm00443039_m1 | P16INK4a: Mm00494449_m1 |
| Guinea Pig Taqman Probes: | |
| ACTB: Cp03755210_g1 | GAPDH: Cp03755743_g1 |
| MMP13: APRWJ74 | |

siRNA Serum and Synovial Fluid Stability Assay. To assess stability of the enhanced stabilization chemistry siRNA molecules, stability assays were performed. siRNA (0.1 nmol) in 60% fetal bovine serum in PBS or synovial fluid obtained from an 83-year-old male patient with untreated, active osteoarthritis was incubated at 37° for 0-24 h, then resolved on a 2% agarose gel in 1×TAE Buffer. Gels were stained with GelRed Nucleic Acid Stain (Biotium) according to the manufacturer's protocol and imaged with UV transillumination.

Size Exclusion Chromatography (SEC) Assessment of Protein Binding in Different Types of Human Synovial Fluid. 1 uM of Cy5-labeled siRNA or Cy5-labeled siRNA<(EG₁₈L)₂ was complexed with 100 uL synovial fluid taken from either a healthy donor (Myocardial infarction death with no history of rheumatic disease, Lee Biosolution, 991-42-S), a donor with untreated osteoarthritis, or a donor with untreated rheumatoid arthritis for 30 minutes. The siRNA-synovial fluid mixtures were then filtered (0.22 um) and injected into an AKTA Pure Chromatography System (Cytiva) with three inline Superdex 200 Increase columns (10/300 GL) for fractionation at 0.3 mL/min using Tris running buffer (10 mM Tris-HCl, 0.15M NaCl, 0.2% NaN3) into 1.5 mL fractions with a F9-C 96-well plate fraction collector (Cytiva). Cy5 fluorescence was measured in fractions (100 uL) in black, clear-bottom, 96-well plates (Greiner-Bio-one REF 675096) on a SynergyMx (Biotek) at a gain of 120, excitation 642/9.0, emission 675/9.0. Albumin-associated fractions were determined by running known protein standards through the SEC system and examining A280 of eluent from each of the fractions and by western blotting/Coomassie blue (Figure S3). For the albumin western blot, gel electrophoresis was performed using 4-20% Mini-PROTEAN® TGX™ Precast Protein Gels, 15-well, 15 μl #4561096 with Tris Glycine SDS running buffer. 9 ul of Samples (constant) were used along with 3 ul of 4×Laemmli Sample Buffer #1610747 with 1.5% of BME. Western blot transfer was done using Invitrogen iblot 2 in accordance with the company protocol. The samples were transferred to nitrocellulose membrane and blocked in Intercept® (TBS) Blocking Buffer. The anti-human albumin antibody (ab 19180, abcam) was used at a 1:1000 dilution and the anti-goat secondary at 1:10000 (IRDye® 800CW Donkey anti-Goat IgG Secondary Antibody, Li-Cor).

Cellular Uptake and Confocal Microscopy Study. Cy5-labelled siRNA treatments were prepared at 100 nM concentrations with albumin at a 10×concentration of 1 uM. For microscopy imaging uptake of Cy5-labeled siRNAs, ATDC5 cells were seeded at a density of 15,000 cells/slide onto Lab-Tek II 8-well chamber slides and allowed to adhere overnight in a standard incubator. Cy5-labeled siRNAs were pre-complexed with mouse serum albumin (MSA) at a 10×molar ratio for 30 minutes at room temperature and then added to the ADTC5s in serum-free Opti-MEM media. After a 4-hour incubation, cells were washed with PBS, fixed in 4% PFA, and Imaging was performed on a Nikon Eclipse Ti-0E inverted microscopy base. For quantifying uptake of Cy5-labeled siRNAs, ATDC5 cells were plated at 10,000 cells/well in a 96-well plate overnight. At this time point, cells were treated with annealed Cy5-labeled siRNA (100 nM) for 2 hours in serum-free Opti-Mem or with 1 uM MSA doped in, or with serum-containing media. Cells were then prepared for flow cytometry by washing twice with PBS, harvesting with trypsin, and resuspending in PBS. Mean fluorescence intensity was measured on a Guava EasyCyte (Luminex) after gating over 500 cellular events.

In Vivo Short-term Unilateral Mechanical Loading PTOA Model for Pharmacokinetic Studies vs. Silencing Studies. The PTOA model of noninvasive repetitive joint loading was induced by subjecting the knee joints of mice (anesthetized with 3% isoflurane) to 250 cycles of compressive mechanical loading at 9 N. This procedure was repeated three times per week over a period of two weeks using conditions adapted from previous studies (1, 2). For all pharmacokinetic studies except for the Cy5 longevity experiment (Evans Blue, MSA-Cy5, Cy5-siRNA), C57 mice were mechanically loaded 3 times per week for 2 weeks (6 total loading cycles), only in their left knee joint (right knee joint was healthy) before being injected with treatment. For all silencing and therapeutic studies, C57 mice were mechanically loaded 3 times per week for 1 week (3 total loading cycles) in both knees (bilateral) before being injected with treatment.

Evans Blue Accumulation, Quantification, and Microscopy. A stock solution of 2% (wt/v %) Evans Blue was dissolved in 0.9% sterile saline in a sterile hood. A dose of 200 uL was administered per mouse via tail vein injection; successful injection was observed by an immediate blue skin change. 24 hours after Evans Blue administration, mice were sacrificed and perfused with cold PBS via intracardiac puncture to get rid of any Evans Blue potentially remaining in the vasculature. Hindlimbs were harvested and allowed to air dry overnight in aluminum foil for the removal of water from tissues. Joint tissues were dissected and isolated, weighed, and placed in 500 uL of Formamide at 55 degrees Celsius for 24 hours to extract the dye. 100 uL triplicate aliquots of extracted Evans Blue/formamide mixture for each sample was then transferred to a 96-well plate, and Evans Blue absorbance was measured at 610 nm using a plate reader (Tecan, Mannedorf, Switzerland). A standard curve of ng Evans Blue was created to determine ng Evans Blue/mg of tissue and graphed as unloaded knees vs. loaded knees and healthy vs. K/BxN serum transfer paws. In addition, some knee joints and paws were flash frozen in liquid nitrogen and used for cryosectioning and confocal imaging of Evans Blue.

Exogenous Mouse Serum Albumin vs. Poly(ethylene glycol) and siRNA, siChol, and SiRNA<(EG18L)2 Cy5 Fluorescence Pharmacokinetic Studies. Mouse serum albumin (Sigma) and 40 kDa PEG (Sigma) were labelled with a Cy5® Conjugation Kit (Fast)—Lightning-Link® (Abcam) and purified. Cy5 labelling efficiency was determined by using a plate reader (Tecan, Mannedorf, Switzerland). Mice were loaded utilizing the short-term model described above. After the last loading cycle, equal mols of MSA-Cy5 and PEG-Cy5 were IV injected via tail vein at a matched dose of 3 mg/kg which was close to the siRNA molar dose (2.39023E-06 mols=1 mg/kg) used in the PK study. In the siRNA PK study, mice were injected with a matched dose of 1 mg/kg via tail vein IV injection (Cy5-siRNA, Cy5-siChol, siRNA<(EG18L)2). Mouse hindlimbs were shaved/nared and intravitally imaged Cy5 fluorescence over time using an IVIS Lumina III intravital imaging system (Caliper Life Sciences, Hopkinton, MA). For IVIS image analysis, fixed regions of interest (ROIs) were drawn around both the right and the left knee joints and around all organs. A pre-injection (blank) image was taken followed by a time 0 (T0) image directly after intra-articular injection. The blank reading was used for background correction of all images at all timepoints. Animals were euthanized at 24 hours. The skin was then removed from the legs (which provides better sensitivity than intravital imaging), and the knee joints were endpoint imaged ex vivo for remaining Cy5 fluorescence. Organs were also removed at this time and imaged alongside the hindlimbs for biodistribution measurements. Legs were then snap frozen in liquid nitrogen and stored at −80° C. until processing for cryosectioning. A subset of knee joints were dissected into cartilage and synovial tissues for imaging these tissues directly under the IVIS.

Tissue Retention Longevity of Systemic Local Cy5-siRNA (EG₁₈L) 2 in Mechanically-loaded, Arthritic Knee Joints vs. Healthy Knee Joints and in K BxN Inflamed Paws. To determine the in vivo longevity of Cy5-siRNA<(EG₁₈L)₂ retention, mice underwent the same loading regimen for silencing/therapeutic studies described above and were loaded 3 times (1 week) in unilateral fashion (left knee loaded, right knee healthy). Dosing followed therapeutic/knockdown studies with 10 mg/kg being injected systemically (intravenous, IV) and 1 mg/kg being injected locally (intra-articular, IA). Intravital IVIS images were taken throughout the 30 day time course. At 30 days, mice were sacrificed, and knee joints/organs were imaged ex-vivo and flash frozen and stored for cryohistology. 10 mg/kg Cy5-siRNA<(EG₁₈L)₂ was also injected 4 days after K/BxN serum transfer and was also imaged similarly across the entire time course and at the endpoint.

Systemic siMMP13, siMMP13-Chol, and siMMP13 (EG₁₈L)₂ Knockdown Comparison Study and siMMP 13 (EG₁₈L)₂ Longevity of Silencing Study. To determine the efficacy of siMMP13<(EG₁₈L)₂ compared to unconjugated siMMP13 or siMMP13 conjugated to cholesterol, mice underwent the same loading regimen described above and were loaded 3 times (1 week) in both knees for a total of 9N of force and 250 cycles before being IV tail vein injected with either a 10 mg/kg matched dose of either siMMP13<(EG₁₈L)₂, siMMP13, or siMMP13-Chol. After injection, mechanical loading continued with the same frequency to maintain elevated MMP13 expression in the joints. Mice were then euthanized at 5 days post-injection and knee joint tissue harvested for gene expression analyses. Using the same protocol, 5 mg/kg vs. 10 mg/kg of siMMP13<(EG₁₈L)₂ was compared to decide what dose to use for the longevity of silencing and therapeutic studies. For the longevity of silencing study, the same protocol was used with 10 mg/kg siMMP13<(EG₁₈L)₂ IV treatment and animals were taken down at 5 days, 10 days, 20 days, or 30 days after injection whilst mechanical loading was still occurring. At the 10 day timepoint, kidneys and livers were analyzed for MMP13 expression via qRT-PCR and MMP13 IHC. The extracted knee joint RNA 10 day time point was also used for the Nanostring analyses described further below.

siMMP13 (EG₁₈L) 2 In Vivo Route of Delivery Knockdown Study. Three different routes of delivery were interrogated for siMMP13<(EG18L)2: Intravenous (tail vein) (V), intra-articular (IA), and subcutaneous (SubQ). Mice underwent the same loading regimen described above and were loaded 3 times (1 week) in both knees for a total of 9N of force and 250 cycles before being injected with the following doses/routes: intravenous (tail vein) 10 mg/kg, intra-articular (trans-patellar) 1 mg/kg, subcutaneous (in between shoulder blades) 20 mg/kg or 50 mg/kg or 20 mg/kg precomplexed with a 5:1 molar ratio of albumin for 30 minutes before injection (attempt to saturate free lipid tails to prevent interaction with deep subcutaneous tissue/fat).

Histological Cryosectioning and Confocal Microscopy. Limbs were harvested, flash-frozen, stored at −80° C., and embedded into OCT freezing compound. Samples were serial sectioned in sagittal orientation until an adequate depth of the joint was reached for optimal visualization. Cryosections at various depths along the joint were then sectioned sagitally at 20 μm thick, captured utilizing a commercially available polyvinylidene chloride film coated with synthetic rubber cement (http://section-lab.jp/), and placed on a slide. Slides were then fixed in 10% neutral buffered formalin for 5 minutes (some were stained with DAPI), cover slipped with an aqua mount, and imaged on a Nikon Eclipse Ti inverted confocal microscope. Imaging settings were kept constant for imaging of all slides. Whole joint imaging was performed by stitching 4×4 10×images. 20×images were taken for each sample. TD=transmission detector.

Immunofluorescence Staining. For immunofluorescence, all slides (cryosections) were rinsed 3× with PBS, treated in 4% PFA for 10 minutes, rinsed 3× with PBST (1×PBS with 0.5% Triton-X 100), blocked with PBST-5% donkey serum for 1 hour at room temperature, and then treated with the antibody of choice. Antibody treatments utilized here include primary anti-guinea pig MMP13 (1:100) (ARP56350_P050, Aviva Systems Biology) and goat anti-rabbit Alexa Fluor® 488 (ab 150077, Abcam; 1:500) or 1:100 primary Lycopersicon Esculentum (Tomato) Lectin (LEL, TL) DyLight™ 488 (DL-1174-1) that stains for blood vessels. After the corresponding treatment time, slides were washed with PBST, incubated with DAPI, and treated with ProLong Gold Antifade Mountant. Imaging was performed on a Nikon Eclipse Ti inverted confocal microscope. Imaging settings were kept constant across different treatment groups. 20×images were performed with serial magnifications.

Immunohistochemical Staining. Slides (paraffin) were placed on the Leica Bond-RX IHC stainer (Leica Biosystems Inc., Buffalo Grove, IL). All steps besides dehydration, clearing and coverslipping are performed on the Bond-RX. Slides are deparaffinized. Heat induced antigen retrieval was performed on the Bond-RX using their Epitope Retrieval 1 solution for 20 minutes. Slides were incubated with anti-MMP13 (cat #Ab39012, Abcam, Cambridge, MA) for 1 hour at a 1:750 dilution or with 1:500 C1,2C (Col 2 3/4Cshort) Polyclonal Rabbit Antibody (Product Number 50-1035, Lot R2699, IBEX Pharmaceuticals). The Bond Refine Polymer detection system was used for visualization. Slides were the dehydrated, cleared and coverslipped. Antibody validation was performed on various mouse organs and positive (targeting primary) and negative (isotype primary) controls were performed. Slides were imaged using a Leica SCN400 Slide Scanner (Leica Biosystems Inc., Buffalo Grove, IL) and 20×, 40×, and 80×images were taken of the cartilage surface, synovium, and meniscus for each slide in each treatment group.

Post-traumatic Osteoarthritis (PTOA) Mechanical loading Model and In Vivo Therapeutic Study. We next benchmarked siMMP13<(EG₁₈L)₂ against an FDA approved sustained release corticosteroid formulation (Zilretta), a non-clinical MMP13 small molecule antagonist CL-82198, and a pan-MMP inhibitor, Marimastat. Marimastat was given intraperitoneally at 10 mg/kg, a dose that has been shown to growth plate effects (3); CL-82198 was given intraperitoneally at 10 mg/kg, a dose that has been shown preclinically to provide joint protection with every other day treatment (4); FDA-approved Zilretta was dosed at 4 mg/kg per knee (8 mg/kg total per animal), a dose species-scaled from successful rat preclinical studies (5). CL-82198 and Marimastat were added to the study design as benchmarks against small molecule MMP inhibitors, while Zilretta was considered a clinical gold standard. A non-gene targeting albumin-hitchhiking siRNA construct (siNEG<(EG₁₈L)₂) was used as a control. siMMP13<(EG₁₈L)₂ and siNEG<(EG₁₈L)₂ were injected biweekly (2× throughout the study). A mechanical overload model was used whereby the knee joints of 6-month-old C57BL/6 mice were cyclically loaded using an ElectroForce 3100 test frame (TA Instruments; New Castle, DE). The mice were anaesthetized, and the knee joints were positioned in flexion at 140° with the tibia approximately vertical and placed directly under the loading point (1). The cyclic loading regimen utilized 250 cycles at 9 N force and was done 3 times per week. Loading started 1 week before the first injections for a total of 5 weeks (4 weeks post first injection) until tissues and blood were harvested. Knee joint hyperalgesia was quantified using a hand-held algesiometer (Bioseb SMALGO: SMall animal ALGOmeter). The tip of the pressure applicator was pressed to the medial aspect of the knee joint with increasing force until a nociceptive response occurred (struggle or vocalization). An average triplicate threshold force was calculated for each limb. 24 Hours before sacrifice, mice were injected with mAbCII680 and MMPsense750 fast (NEV10168, PerkinElmer, Waltham, Massachusetts, USA) and knee joints were then imaged on the IVIS. Extracted blood/serum was then used in a C2C assay (IBEX Pharmaceuticals) to determine in vivo collagen degradation.

K/BxN Serum Transfer Model of Inflammatory Arthritis Therapeutic Study. K/BxN

transgenic mice and female B6.I-Ag7 mice were intercrossed to generate F1 that spontaneously developed arthritis beginning at about 4 weeks of age and lasting for >20 weeks. To induce STA, 8-week-old C57BL/6 mice were challenged with 200 μL of K/BxN serum delivered by intraperitoneal (IP) injection to induce arthritis (Day 0)

For Evans Blue, MSA-Cy5, and Cy5-siRNA, Cy5-siChol, and Cy5-siRNA<(EG₁₈L)₂ delivery studies, respective treatments were given at day 4 after K/BxN serum transfer. Treatments were followed intravitally by IVIS analysis, and ex-vivo joint and organ analyses was done 24 hours after injection. Cryosections of the joints were then performed (like the PTOA pharmacokinetic studies described above).

For the therapeutic study, treatment injections were performed on day 2 after serum transfer: intravenous (tail vein) 10 mg/kg siMMP13<(EG₁₈L)₂, intraperitoneal 10 mg/kg C1-82198, intraperitoneal 10 mg/kg methylprednisolone (Solu-medrol). Disease activity (clinical score and ankle thickness) and pain sensitivity (algometry) were assessed every two days for 10 days. 24 Hours before sacrifice, mice were injected with mAbCII680 and MMPsense750 fast (NEV10168, PerkinElmer, Waltham, Massachusetts, USA) and knee joints were then imaged on the IVIS. Mice were then sacrificed, and blood/serum and tissues (organs, hindpaws, forepaws, knees) were collected.

The clinical score was assessed on a scale of 0 to 3 (0, no swelling or erythema; 1, slight swelling or erythema; 2, moderate erythema and swelling in multiple digits or entire paw; and 3, pronounced erythema and swelling of entire paw; maximum total score of 12)(6). The change from baseline in ankle thickness was determined daily by dial calipers (around ankle malleoli), and an average change in the ankle thickness was determined for each hind paw measurement. Knee joint hyperalgesia was quantified using a hand-held algesiometer (Bioseb SMALGO: SMall animal ALGOmeter). The tip of the pressure applicator was pressed to the medial aspect of the ankle joint (medial malleoli) with increasing force until a nociceptive response occurred (struggle or vocalization). An average triplicate threshold force was calculated for each limb.

Guinea Pig Anterior Cruciate Ligament (ACLT) Model and Systemic siMMP13<(EG₁₈L)₂ Study. 3-month-old male Dunkin Hartley guinea pigs underwent left knee ACLT surgery. The methods for this procedure have been adapted from Jimenez et al. (7). Surgery was performed under direct visualization. For the surgery, a medial longitudinal parapatellar incision was be made over the anterior knee to expose the patellar tendon in anesthetized and surgically prepped guinea pigs. The patella was everted, knee placed in flexion, and the ACL incised. After confirming anterior laxity in the joint, the site was closed with the joint capsule continuously sutured. Ketoprofen was given immediately before surgery and every 24 hours for 3 days post-surgery. 1 week after surgery, 10 mg/kg siMMP13<(EG₁₈L)₂ was injected intravenously (lateral saphenous vein). Animals were taken down 1 week after the injection. For knee joint visualization and organ biodistribution purposes 1 mg/kg IV Cy5 siRNA<(EG₁₈L)₂ was also injected 24 hours before takedown.

Blood Chemistry and Luminex Multiplex Analyses. Whole blood was collected in EDTA-coated tubes or spun down at 1,000×g for 10 min at 4° C. to isolate serum. Samples were then submitted to the Vanderbilt Translational Pathology Shared Resource for chemistry analyses. For Luminex, samples were submitted to the Vanderbilt Hormone Assay & Analytical Services Core.

Paraffin Embedding and Staining Histology. All joints used for histology were fixed in 10% formalin for 1 week, and micro CT analysis was performed in 70% ethanol after fixation. Samples were then decalcified in 20% tetrasodium EDTA for 4 days (exchanging once at 2 days) or until soft to the touch, rinsed with water, and then placed in 70% ethanol at 4° C. until paraffin-block embedded and sectioned. Embedding of tissues for coronal sectioning was aided by manually slicing the patella away such that the anterior aspect of the joint could be positioned facing down in the embedding tray, i.e., “stifled”. Sections were cut 5 μm thick in the coronal orientation, starting posterior to the patella and sectioning fully through the joint. At least two sections in the loading plane were chosen from each joint for Hematoxylin and Eosin and Toluidine Blue staining that were used in subsequent OA/RA severity scoring. For the K/BxN STA study, hindpaws and forepaws were sectioned sagitally with knee joints being sectioned coronally; they were also stained with Hematoxylin and Eosin and Toluidine Blue for scoring purposes. Positive staining controls of the mouse skin containing mast cells were done for Toluidine Blue.

MicroCT. All microCT analysis was performed with the ScanCo μCT-50 (Scanco Medical, Basserdorf, Switzerland) and ScanCo software (Scanco USA, Inc., Southeastern, PA). 3D renderings were shown at a consistent density threshold (42.0% of maximum bone density, or 420 per mille). All samples were fixed with formalin and then emerged in 100% ethanol during micro-CT imaging. The scans were collected with 20 μm thick slices, an isotropic 12 μm voxel size, a current/voltage of 114 mA/70 kVp, and an integration time of 200 millisecond. Imaging, contouring, and all sample measurements and analysis were performed on deidentified samples by a treatment-blinded user. Contouring was performed first to encompass all mineralized components of the joints. Contouring was done a second time to segment out mineralization in the soft tissues surrounding the cortical bone, presented as mg hydroxyapatite. 3D renderings (sigma: 1.5; support: 3; threshold: 388) were also generated using the Scanco software. 2D cross-sectional images were identified in each sample that contained the maximal osteophyte length, which was visualized along with soft ectopic mineralization in a similar plane across all treatment groups. Osteophyte length was measured on each sample in both the femur and the tibia, defined as maximal osteophyte length. For the K/BxN serum transfer arthritis model, contouring was performed over the calcaneus. Hydroxyapatite calibration phantoms were used to calibrate bone density values (g/cm3). Parameters reported are bone surface to bone volume (BS/BV), bone fraction (bone volume/total volume; BV/TV), and bone mineral density (BMD; g/cm3).

TABLE 1
(A) Description of OARSI scale scoring for Safranin-O/Fast Green stained slides of
femoral/tibial cartilage plateaus (left); (B) Criteria for scoring
of H&E-stained slides to assess overall joint by the DJD scale (right).

| Severity | OARSI | Degenerative Joint Disease (DJD) | |
|---|---|---|---|
| Score | Scale | Scale | |
| 0 | Normal | Within | mild DJD as a feature of expected age-related |
| | | normal | change (e.g., attenuation of articular cartilage and |
| | | Limits | proteoglycan loss) |
| 1 | Loss of SO staining, | Moderate | articular cartilage degeneration |
| | articular cartilage | | beginning of secondary pathology |
| | thinning; no defects | | synovitis |
| | | | joint capsule fibrosis |
| | | | meniscal metaplasia |
| 2 | 1 + fibrillation or | Marked | metaplasia and/or fragmentation of one |
| | pyknotic articular | | meniscus |
| | chondrocytes | | osteophyte formation |
| | | | synovitis or hyperplasia |
| 3 | 2 + loss of articular | Severe | metaplasia and/or fragmentation of both |
| | cartilage < 50% (e.g., | | menisci |
| | erosion, flap, or callus) | | eburnation (total loss) of at least 1 |
| | | | articular surface |
| | | | osteophyte formation |
| | | | advanced synovitis, hyperplasia |
| 4 | 3 + fragmentation and | Extreme | most advanced from severe category |
| | fissuring in 1 + articular | | epiphyseal osteolysis |
| | plateau | | collapse of joint space |
| 5 | 4 + fragmentation & | N/A | |
| | fissuring of > 75% | | |
| | cartilage surface | | |
| 6 | Total loss of normal | N/A | |
| | articular cartilage (end- | | |
| | stage) | | |

Histological Scoring. Coronal knee joint sections were stained with Hematoxylin and Eosin and Toluidine Blue for scoring by the Osteoarthritis Research Society International (OARSI) and Degenerative Joint Disease (DJD) scales by at least 2 mid-frontal sections per joint (8). Scoring of de-identified sections was interpreted by a board-certified veterinary pathologist. OARSI scores (0-6 semiquantitative scale; Table 1) were assigned from assessment of the medial and lateral plateaus of the tibia and femur (9). A degenerative joint disease (DJD) severity score (0-4 semiquantitative scale) was determined from H&E stained sections using a semi-quantitative index based on cartilage erosion, subchondral osteosclerosis, synovial/meniscal metaplasia, subchondral osteosclerosis, inflammation, and the growth of osteophytes and meniscal ectopic mineral deposits (10). For the K/BxN serum transfer arthritis model, forepaw, hindpaw, and knee joint sections were also stained with Hematoxylin and Eosin and Toluidine Blue and were scored using 3 scales adapted as previously described (6, 11):

### Inflammation Score (H&E)

0 Absent
Slight/mild inflammation (diffusely located single cells and/or small perivascular infiltrates
1 of neutrophils with lymphocytes and plasma cells)
Moderate inflammation (moderate, multifocal infiltrates of neutrophils with lymphocytes
2 and plasma cells)
Marked inflammation (diffuse, confluent infiltrates of neutrophils with lymphocytes and
3 plasma cells)

### Bone Erosion Score (H&E)

0 None/normal
1 Minimal/sparse (small areas of resorption, hard to identify)
2 Mild (mild but multifocal resorption of cortical and/or trabecular bone)
3 Moderate (resorption of cortical and/or trabecular bone; not full thickness)
4 Marked (full thickness defects in the cortical bone with marked trabecular bone loss)
   Proteoglycan loss (cartilage destruction score) in the cartilage was scored on toluidine blue-stained sections on a scale from 0-4, ranging from fully stained cartilage (score=0) to fully destained cartilage (score=4) with 1=0-25% loss, 2=25-50% loss, 3=50-75% loss, and 4=75-100% loss of staining. Scoring was performed by an histopathologist blinded to the treatment.

nanoString Inflammation Panel Gene Expression Analysis. The Vanderbilt

Technologies for Advanced Genomics (VANTAGE) core processed the RNA (>50 ng purified, normalized RNA per sample) isolated from articular cartilage and meniscal/synovial tissue processed in equal masses. Samples were subjected to VANTAGE core quality control measures before processing using the nanoString nCounter Inflammation (mouse, V2) panel. All hybridizations were incubated for 20 hours, following manufacturer (Nanostring Technologies, Inc., USA) recommended procedures. The nSolver software package was used for comparisons, unsupervised analysis, and gene cluster analysis between groups.

Animal Ethics Statement. All animal experiments described herein were carried out according to protocols approved by Vanderbilt University's Institutional Animal Care and Use Committee (IACUC), and all studies followed the National Institutes of Health's guidelines for the care and use of laboratory animals.

Statistical Methods. Data are displayed as mean plus/minus standard error (unless stated otherwise). Statistical tests employed either one-way or two-way ANOVAs with multiple comparisons test or two-tailed student's T-test between only two groups with α=0.05. All statistical analysis were performed as described in figure captions with GraphPad Prism Version 9.5.0 (525), Nov. 8, 2022 (GraphPad), except for nanoString data analysis with nSolver V3.0 (Nanostring Technologies).

### REFERENCES FOR METHODS OF EXAMPLE 9 (ALL OF WHICH ARE INCORPORATED BY REFERENCE HEREIN IN THEIR ENTIRETY)

1. Poulet B, Hamilton R W, Shefelbine S, Pitsillides A A. Characterizing a novel and adjustable noninvasive murine joint loading model. Arthritis and rheumatism. 2011; 63(1):137-47.
2. Cho H, Pinkhassik E, David V, Stuart J M, Hasty KA. Detection of early cartilage damage using targeted nanosomes in a post-traumatic osteoarthritis mouse model. Nanomedicine: nanotechnology, biology, and medicine. 2015; 11(4):939-46.
3. Renkiewicz R, Qiu L, Lesch C, Sun X, Devalaraja R, Cody T, et al. Broad-spectrum matrix metalloproteinase inhibitor marimastat-induced musculoskeletal side effects in rats. Arthritis & Rheumatism. 2003; 48(6): 1742-9.
4. Wang M, Sampson E R, Jin H, Li J, Ke Q H, Im H-J, et al. MMP13 is a critical target gene during the progression of osteoarthritis. Arthritis Research & Therapy. 2013; 15(1):R5.
5. Kumar A, Bendele A M, Blanks R C, Bodick N. Sustained efficacy of a single intra-articular dose of FX006 in a rat model of repeated localized knee arthritis. Osteoarthritis Cartilage. 2015; 23(1):151-60.
6. Zhou H F, Chan H W, Wickline S A, Lanza G M, Pham C T. Alphavbeta3-targeted nanotherapy suppresses inflammatory arthritis in mice. FASEB J. 2009; 23(9):2978-85.
7. Jimenez P A, Harlan P M, Chavarria A E, Haimes H B. Induction of osteoarthritis in guinea pigs by transection of the anterior cruciate ligament: radiographic and histopathological changes. Inflamm Res. 1995; 44 Suppl 2:S129-30.
8. Bolon B, Stolina M, King C, Middleton S, Gasser J, Zack D, et al. Rodent preclinical models for developing novel antiarthritic molecules: comparative biology and preferred methods for evaluating efficacy. J Biomed Biotechnol. 2011; 2011:569068.
9. Glasson S S, Chambers M G, Van Den Berg W B, Little C B. The OARSI histopathology initiative-recommendations for histological assessments of osteoarthritis in the mouse. Osteoarthritis Cartilage. 2010; 18 Suppl 3:S17-23.
10. Aigner T, Söder S. Histopathologische Begutachtung der Gelenkdegeneration. Der Pathologe. 2006; 27(6):431-8.
11. Choi Y R, Collins K H, Springer L E, Pferdehirt L, Ross A K, Wu C L, et al. A genome-engineered bioartificial implant for autoregulated anticytokine drug delivery. Sci Adv. 2021; 7(36): eabj 1414.

### Results

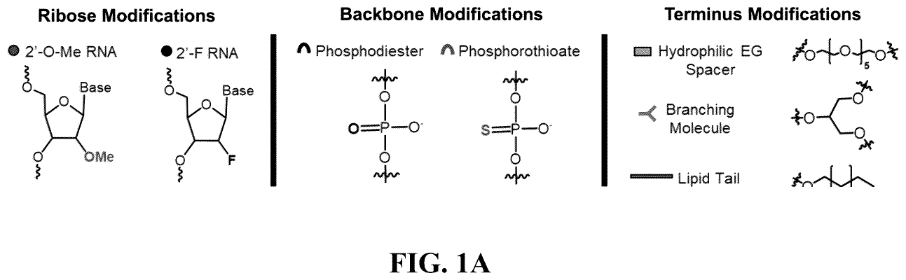

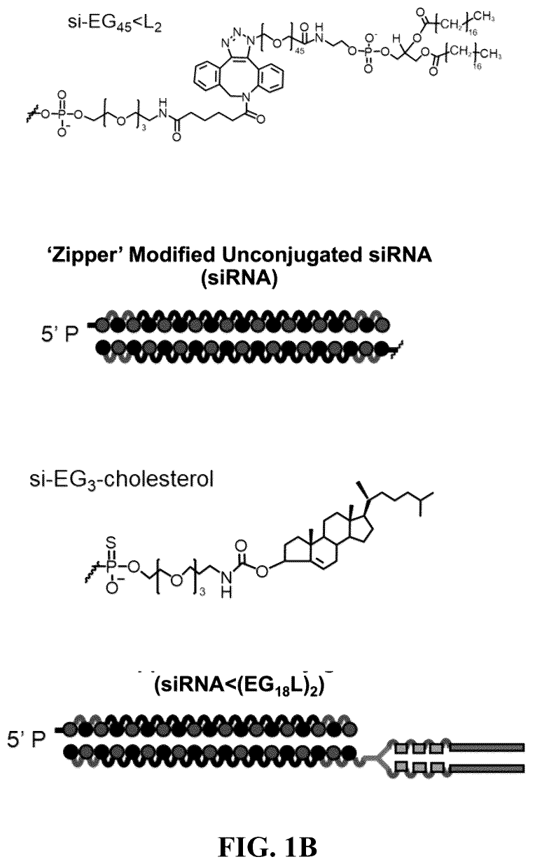

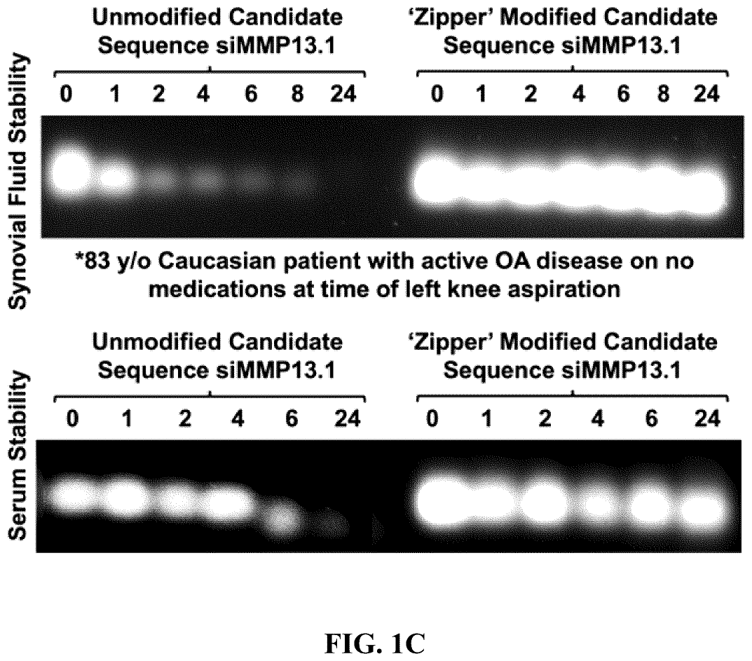

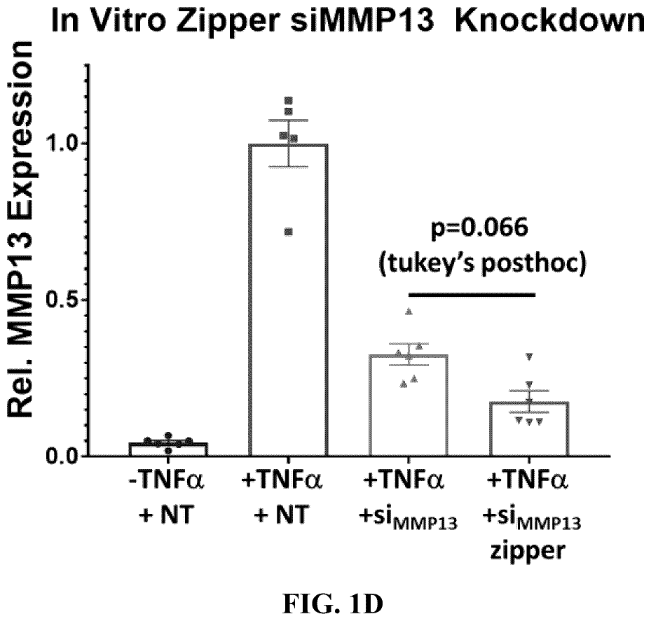

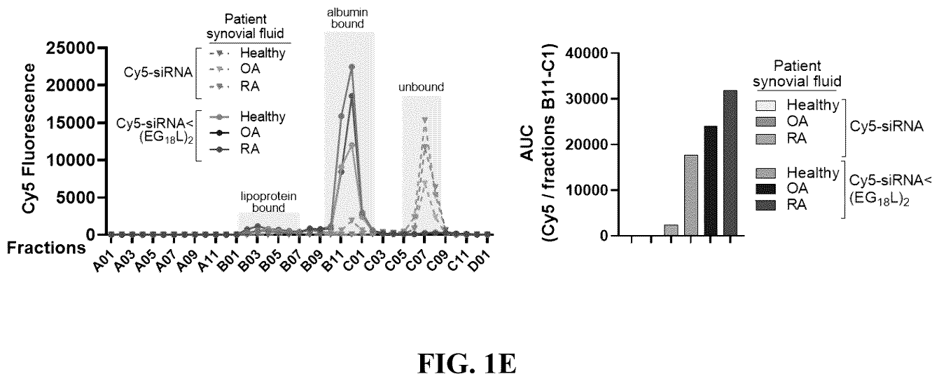

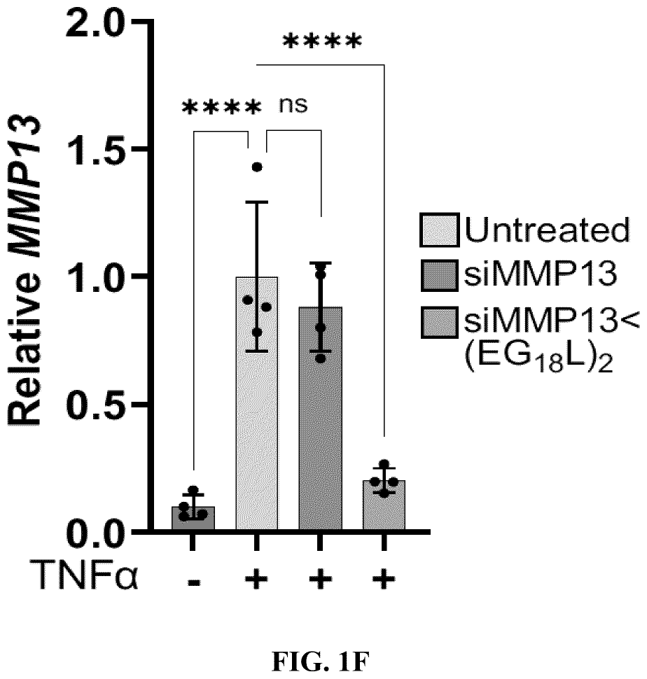

An example conjugate, si<(EG₁₈L)₂, was used to assess silencing capability in inflammatory diseases. Modified nucleotides aided properties of siRNA used herein, such as stability in synovial fluid and in-vitro carrier-mediated silencing (FIG. 1A, FIG. 1B, FIG. 1C, and FIG. 1D). The si<(EG₁₈L)₂ conjugate was also used to assess in vitro carrier-free silencing where it demonstrated knockdown of murine MMP13 (FIG. 1F) and binding to albumin in OA and RA human synovial fluid to a greater amount than in healthy human synovial fluid (FIG. 1E). Meanwhile, regular siRNA without lipophilic albumin-binding moieties did not have any appreciable MMP13 knockdown (FIG. 1F) or binding of albumin in human synovial fluid (FIG. 1E).

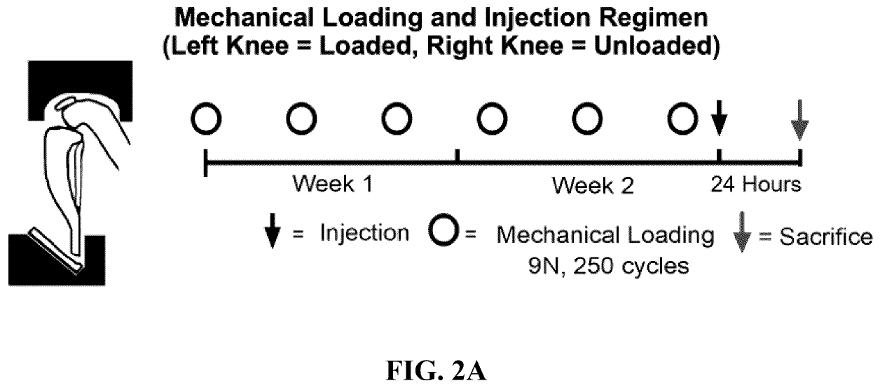

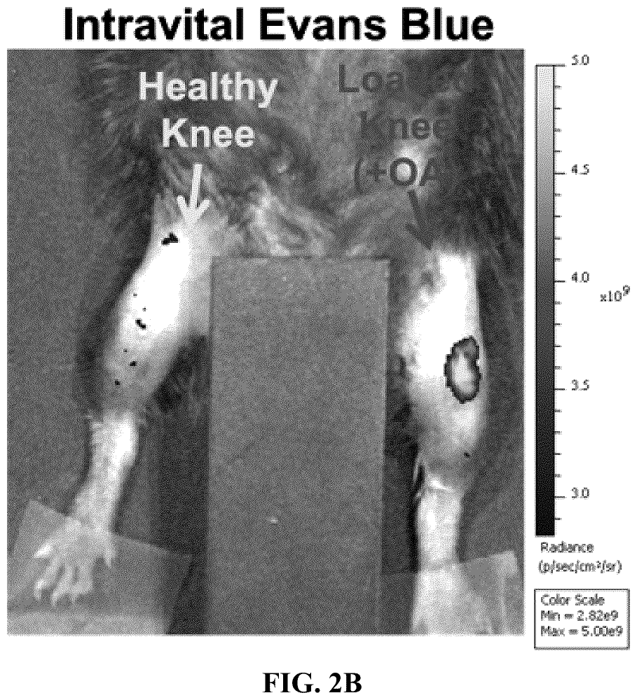

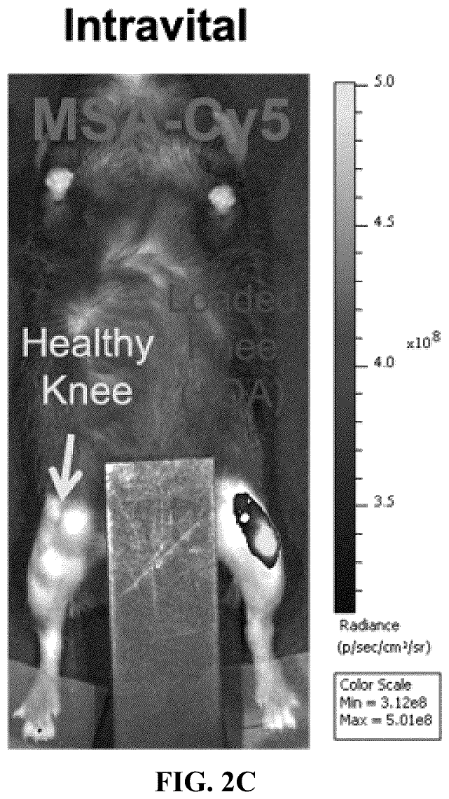

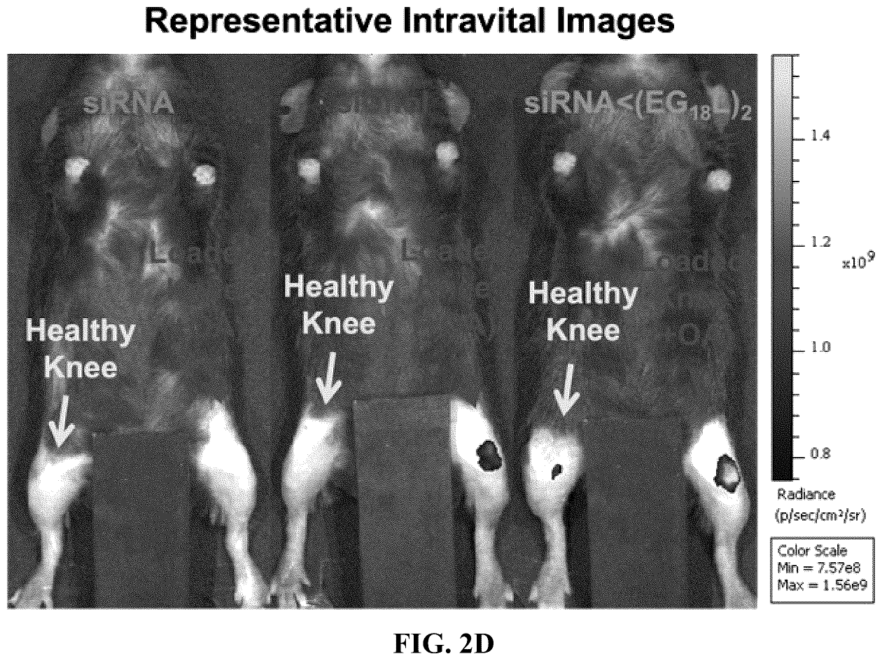

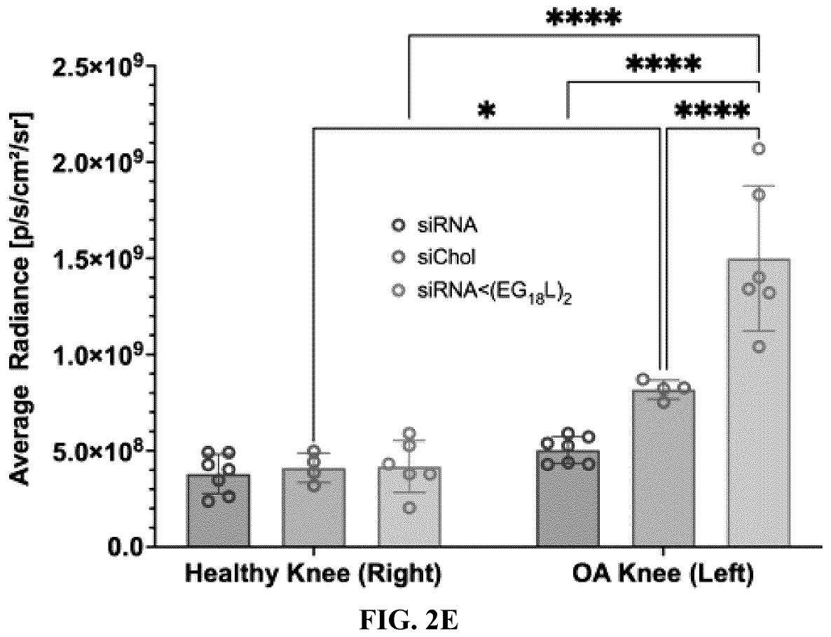

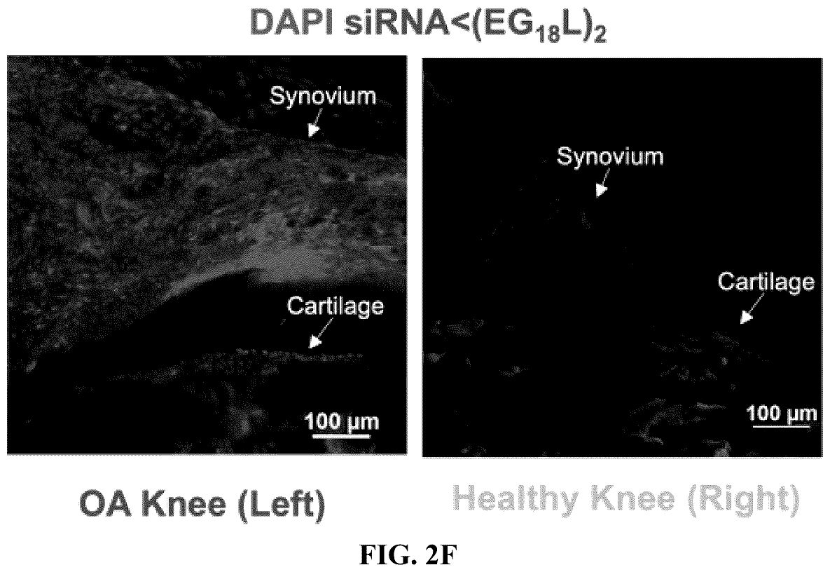

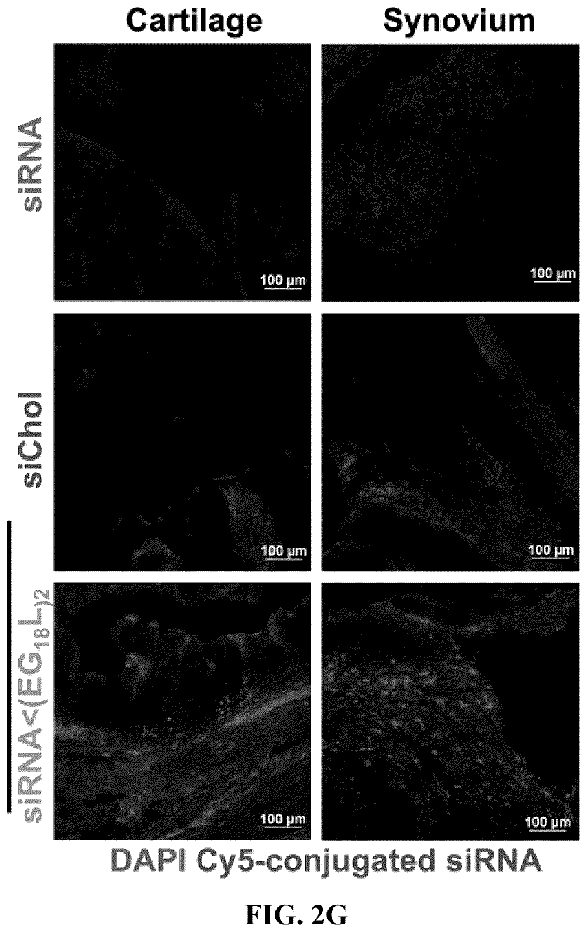

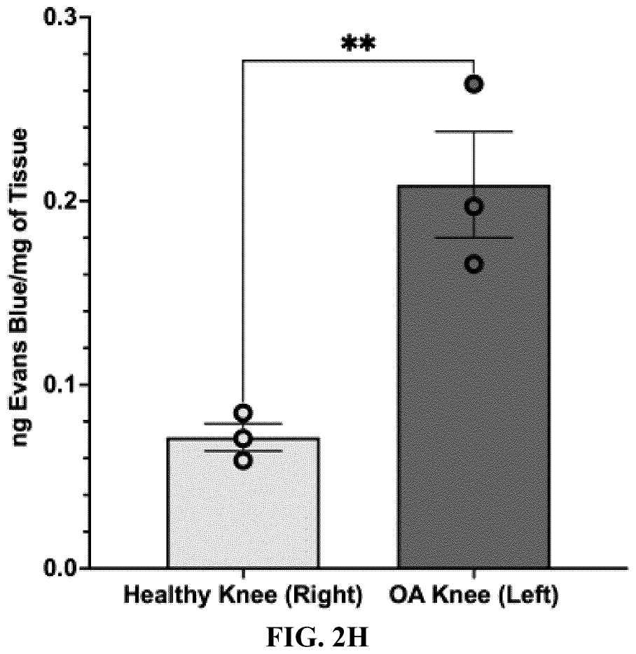

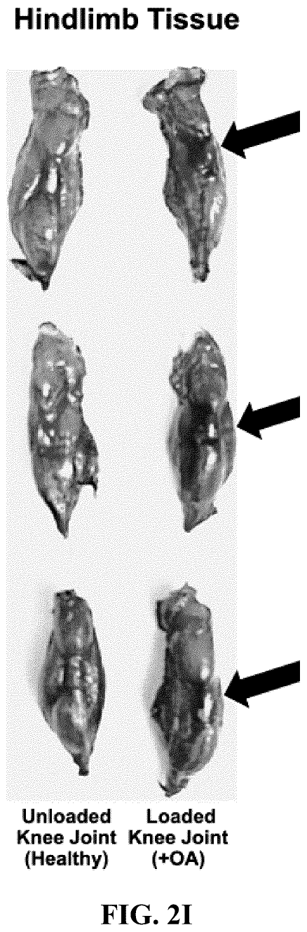

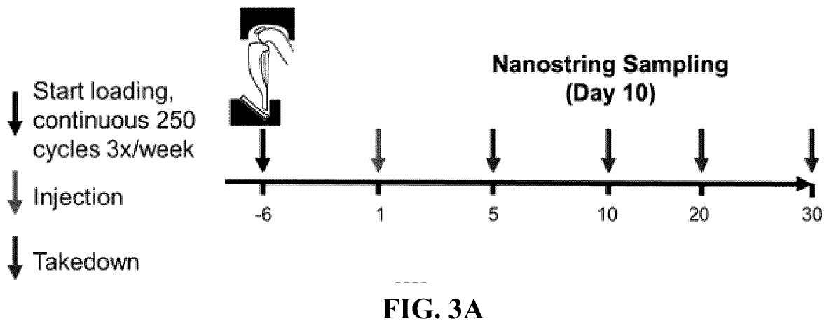

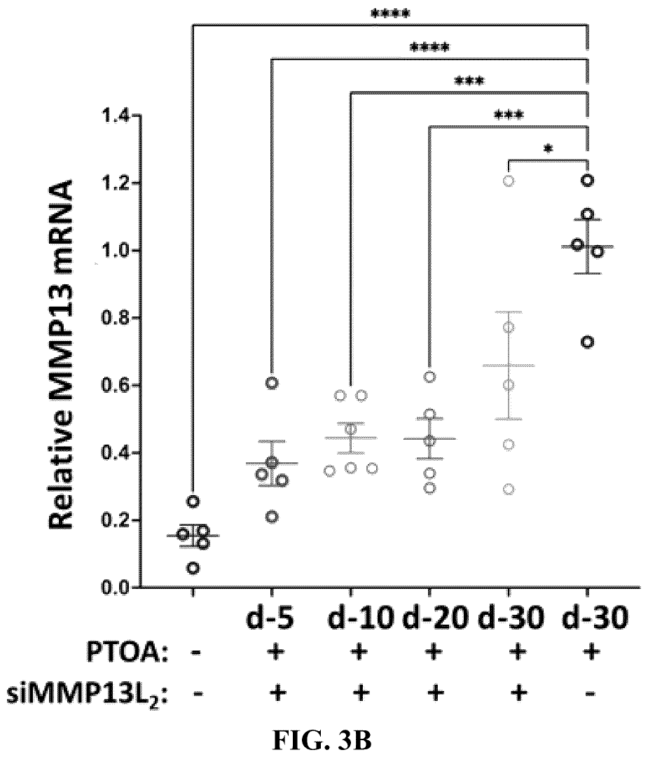

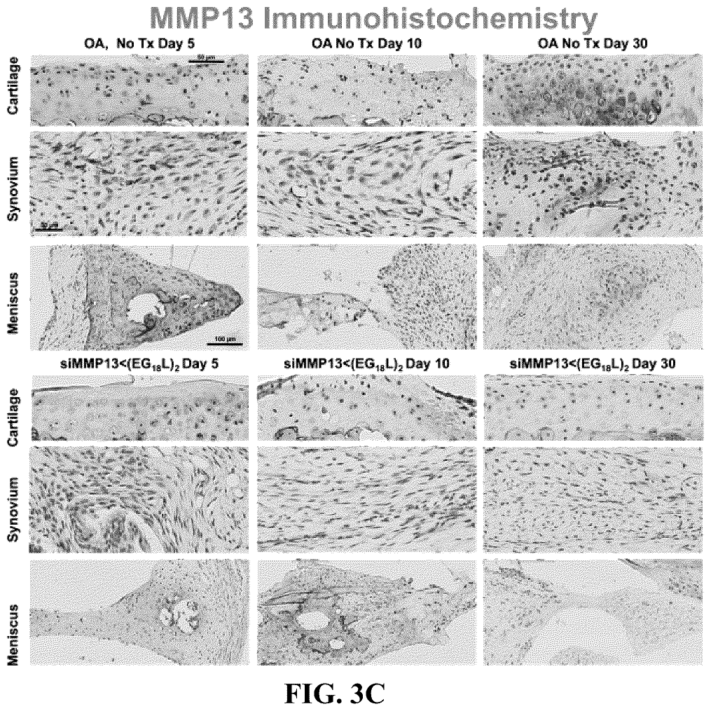

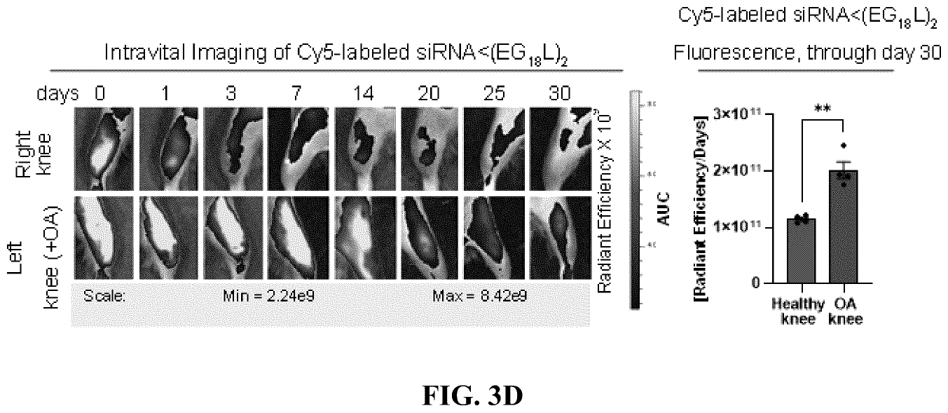

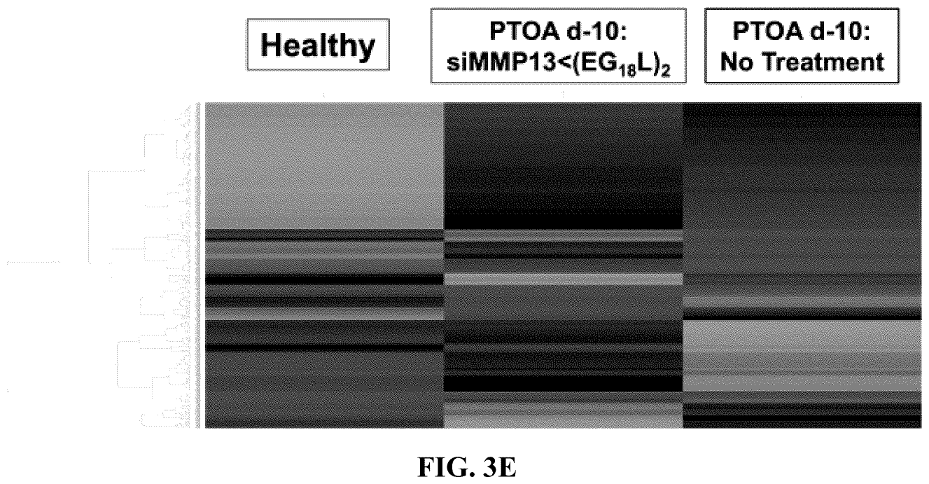

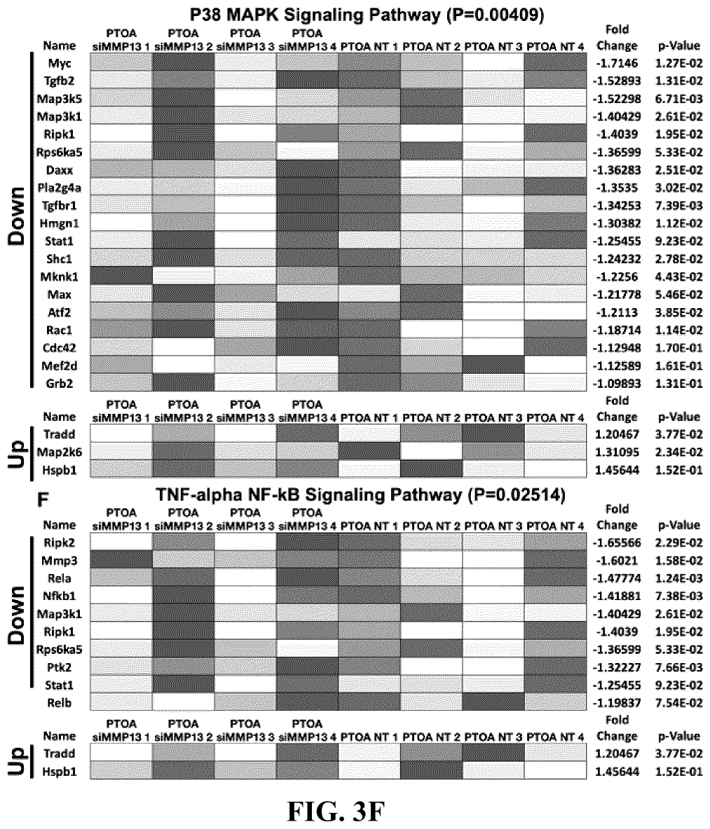

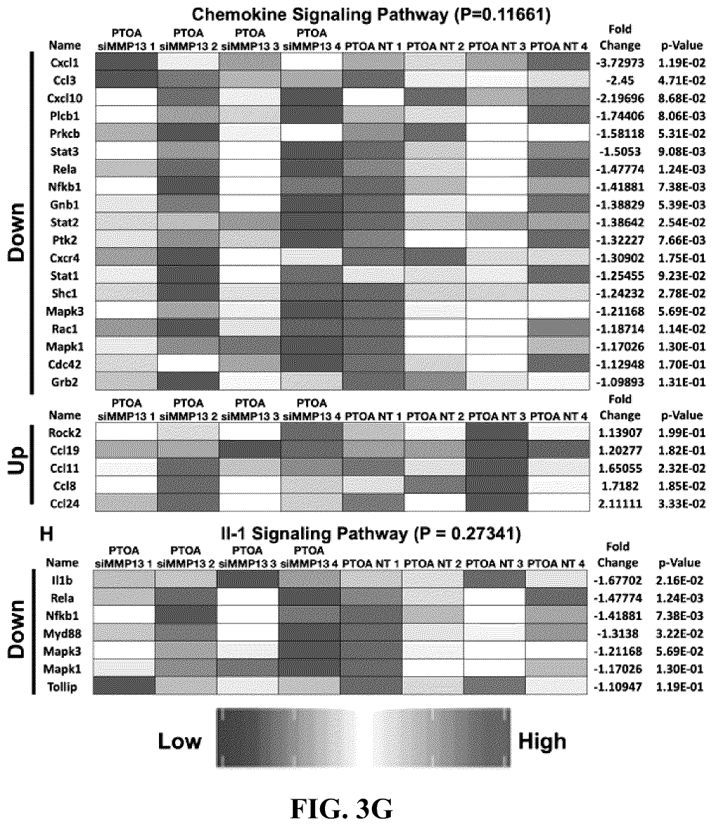

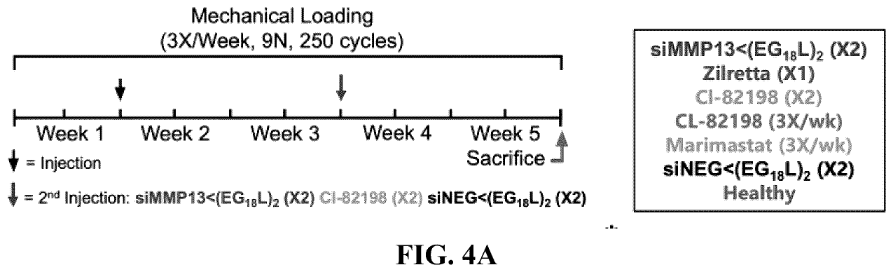

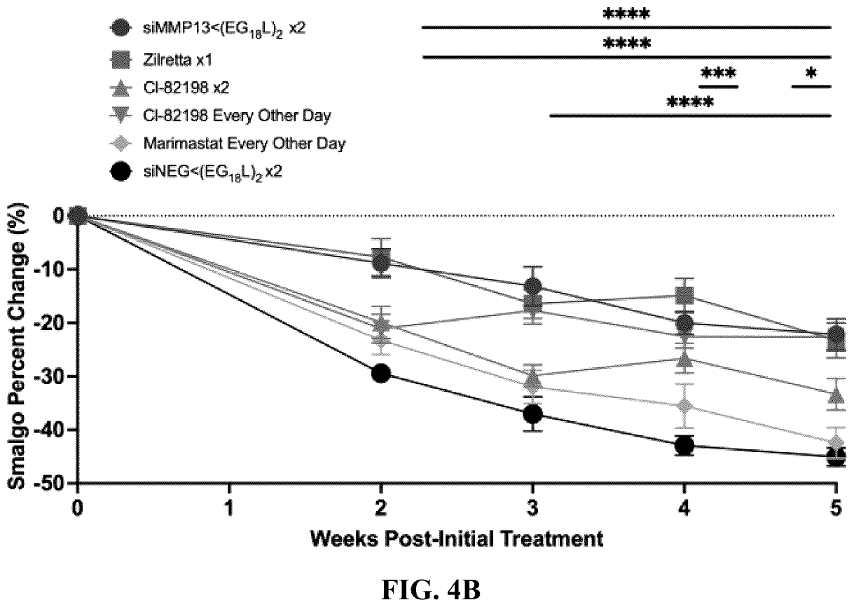

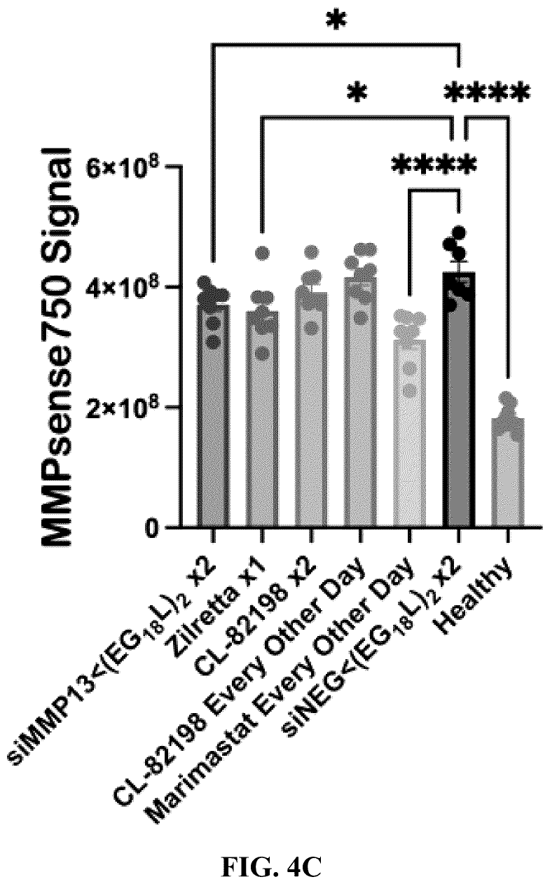

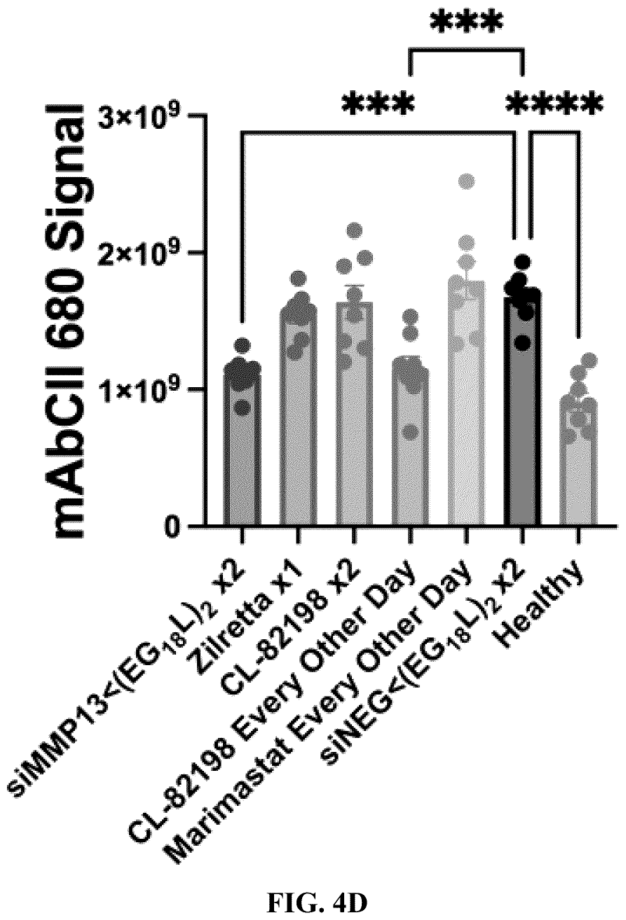

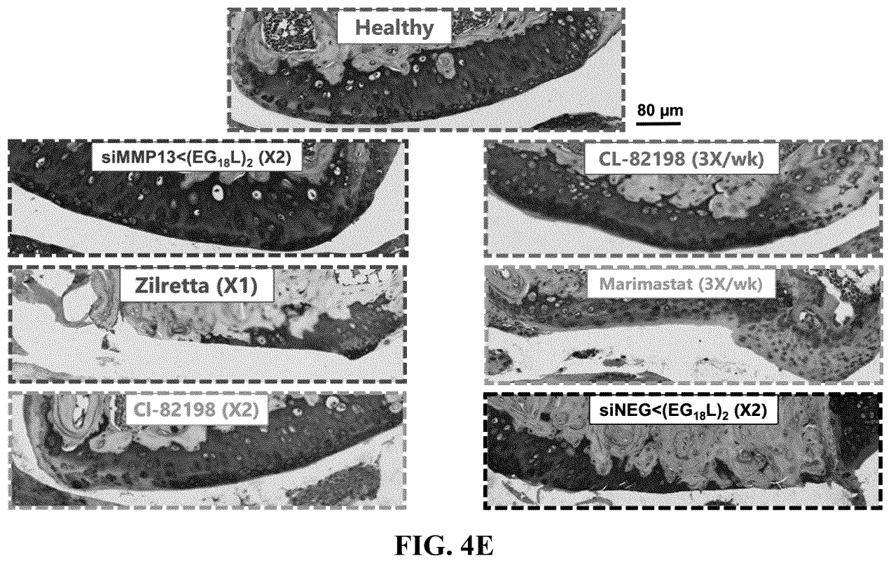

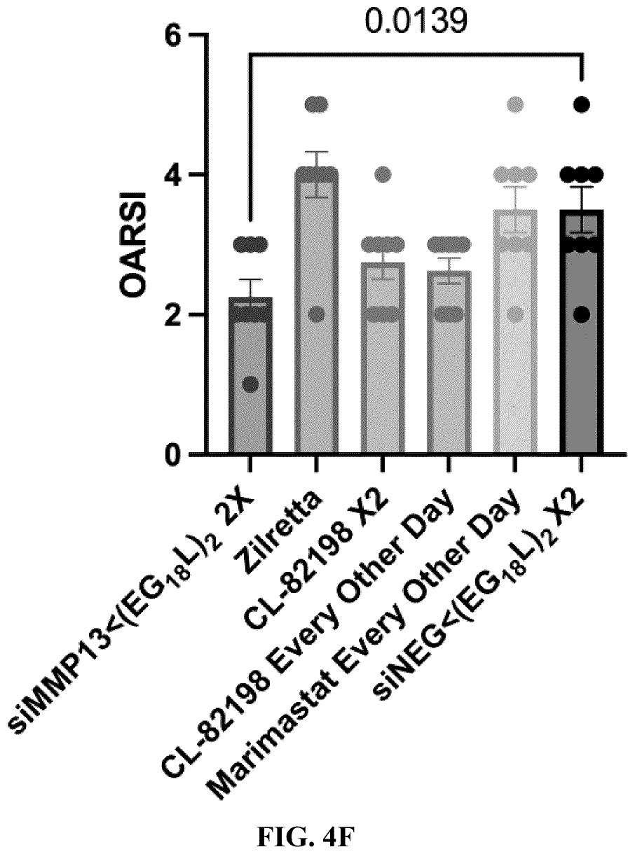

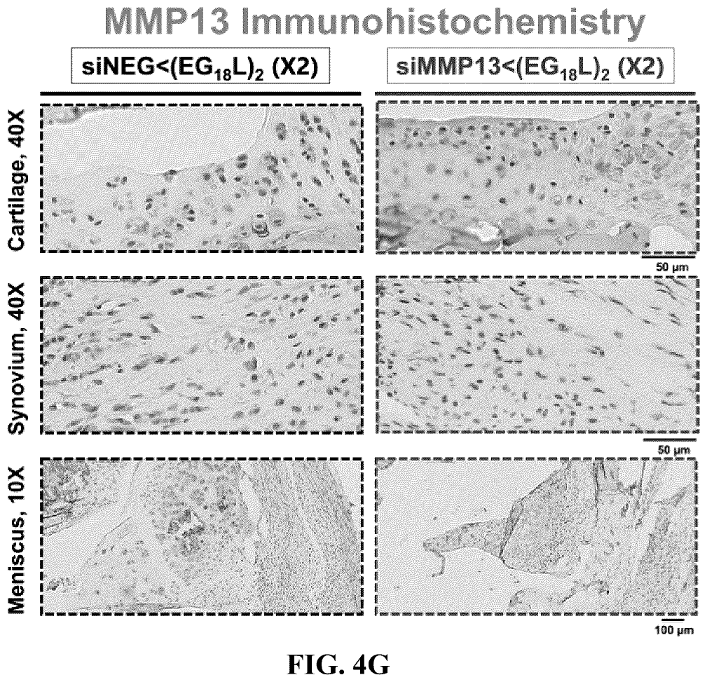

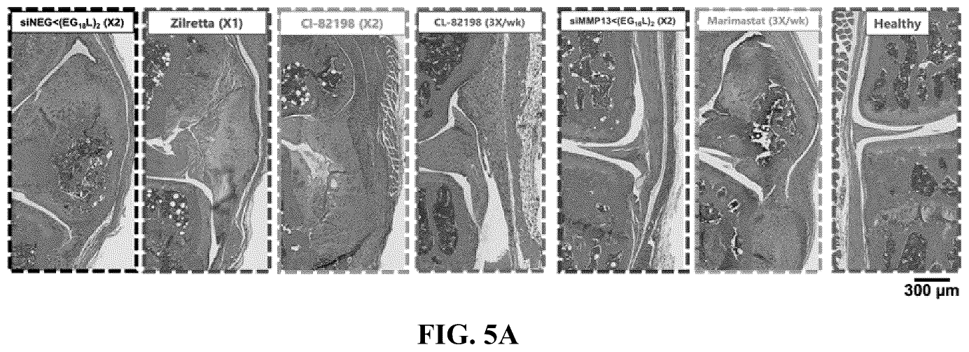

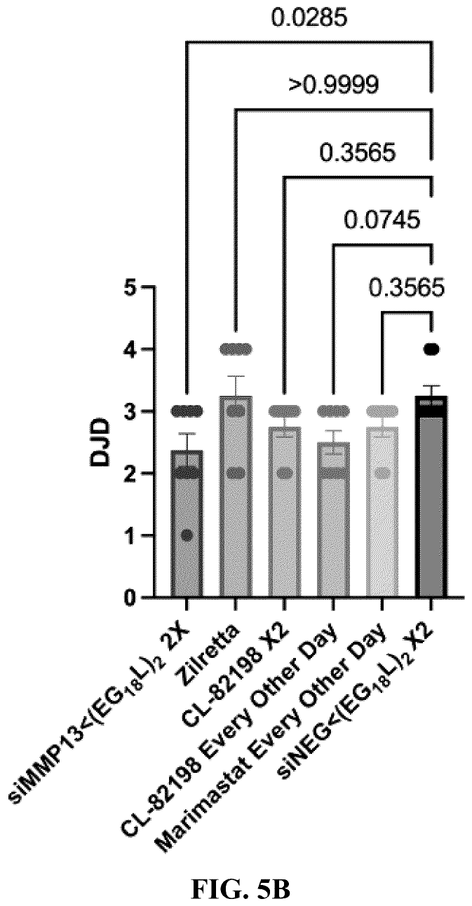

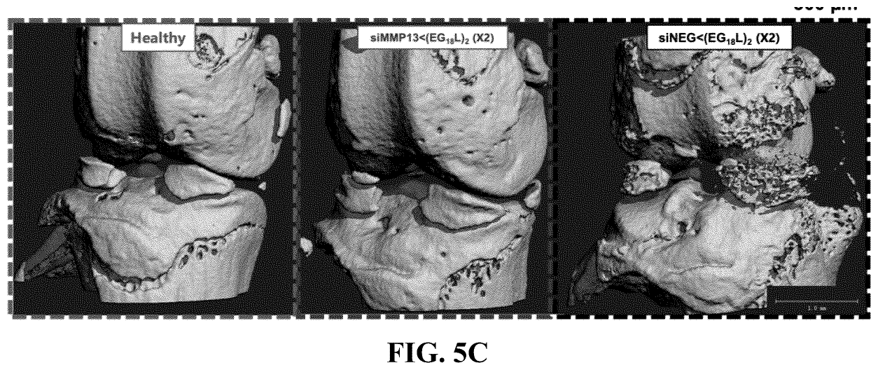

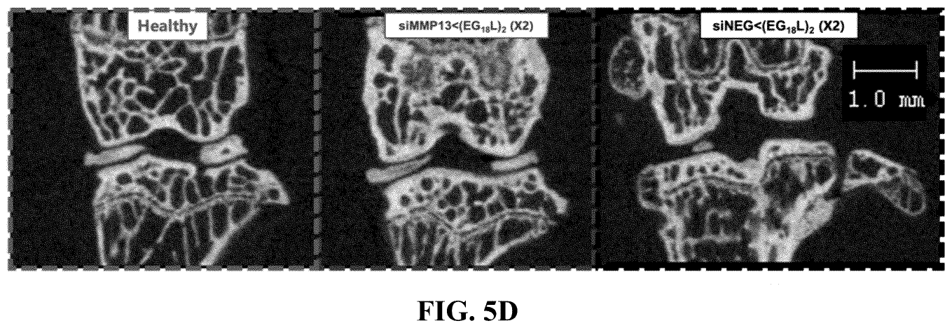

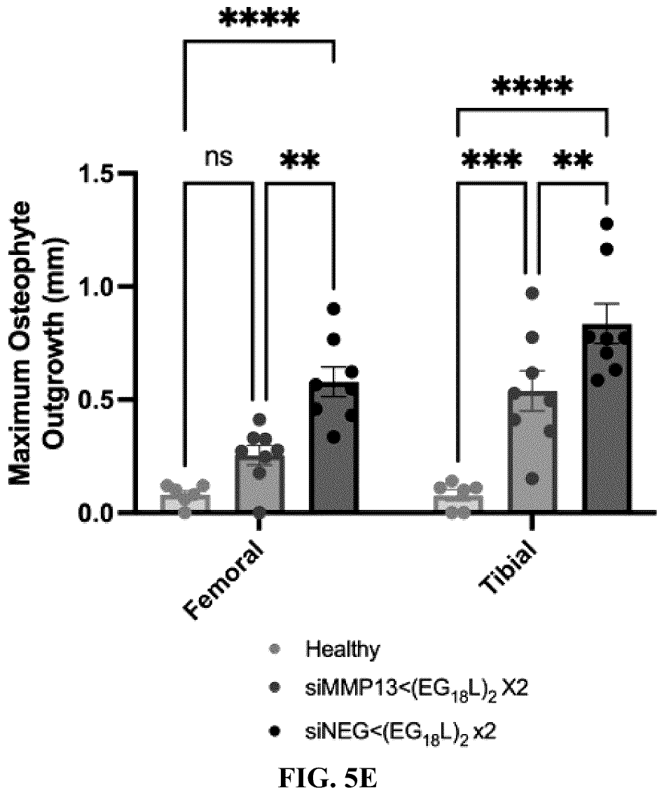

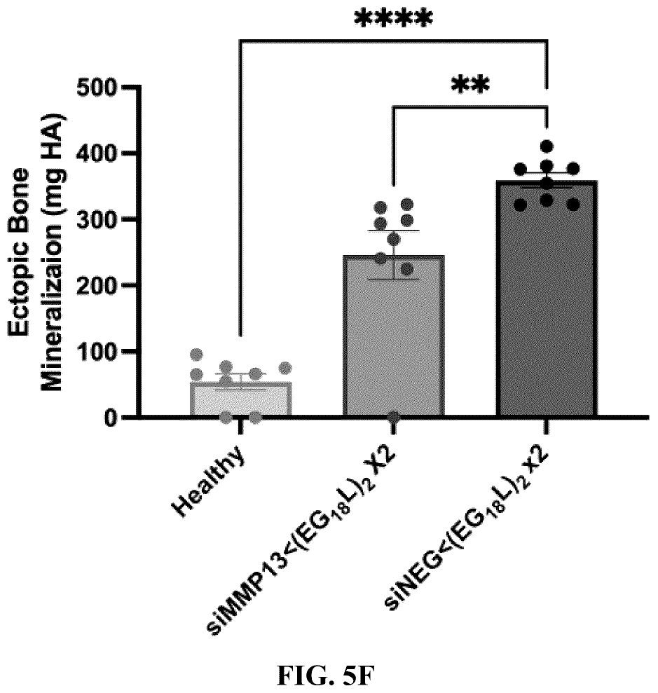

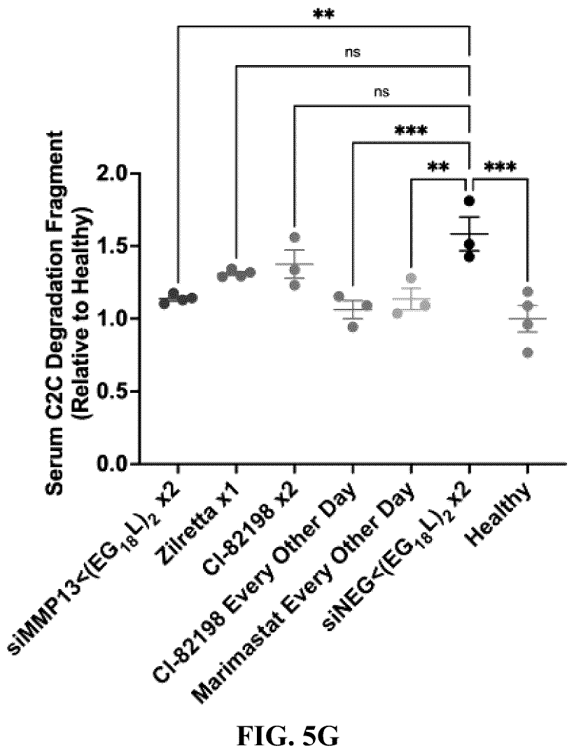

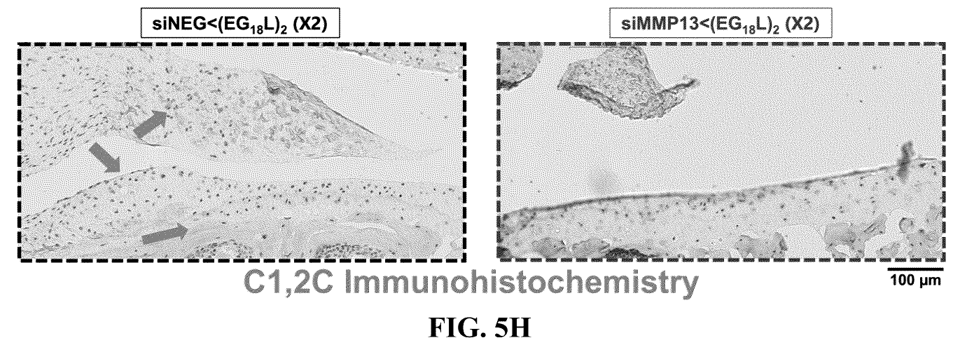

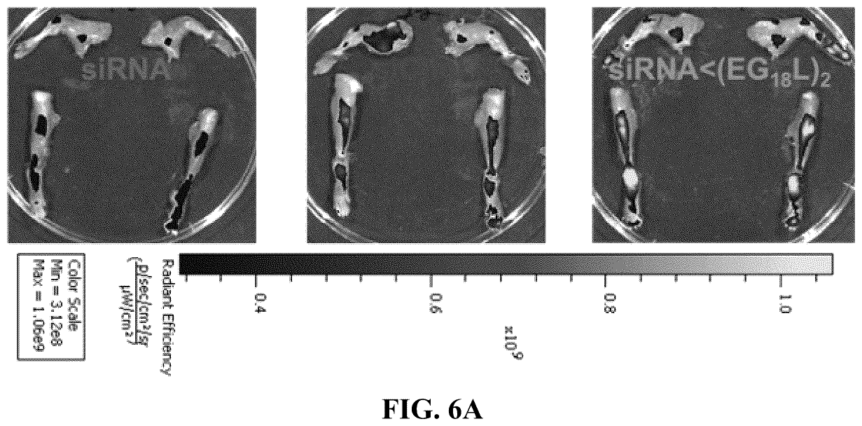

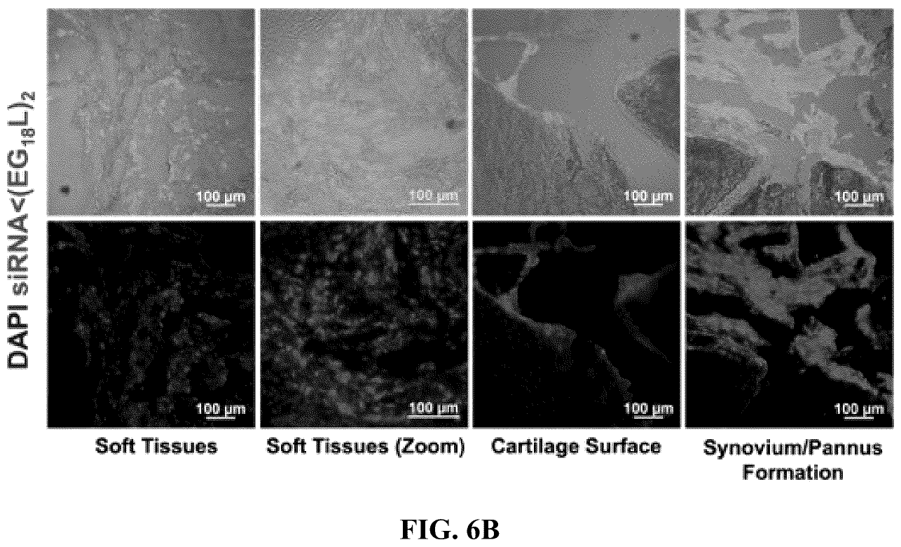

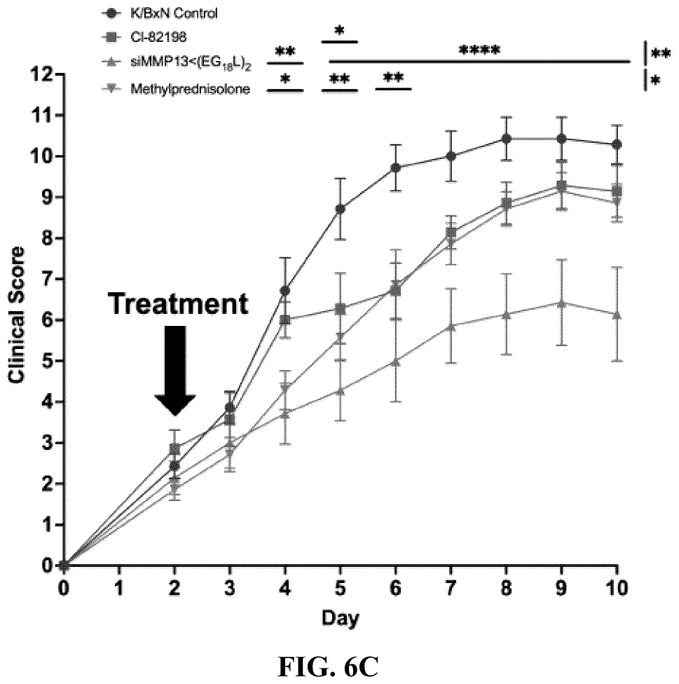

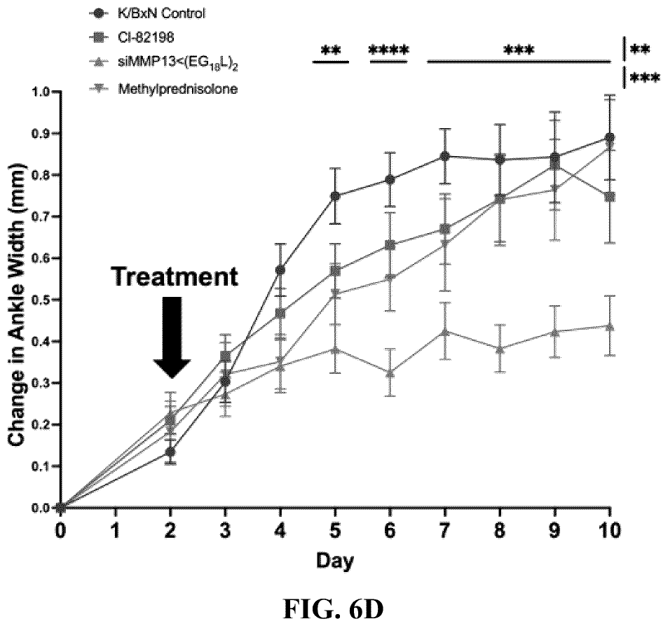

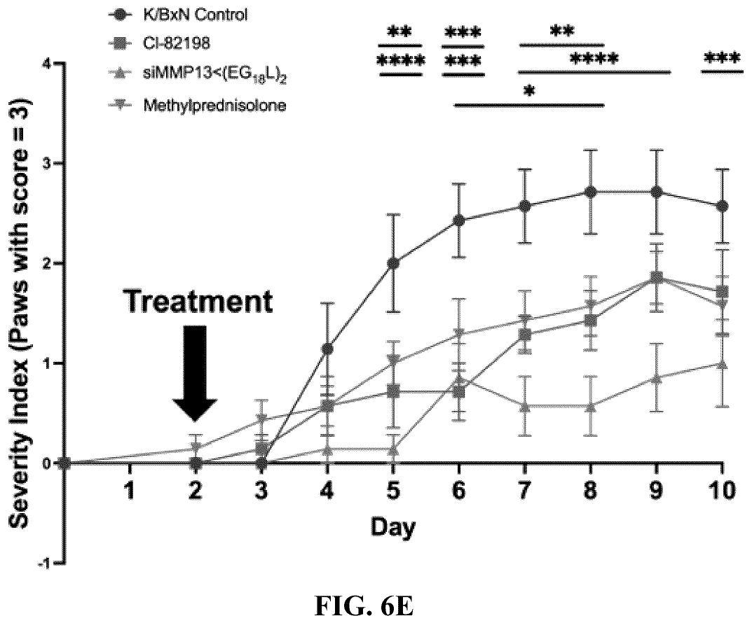

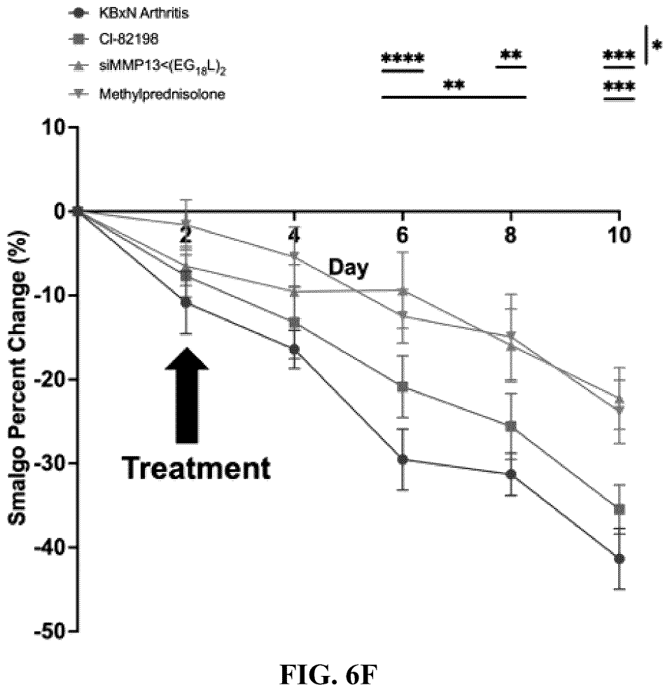

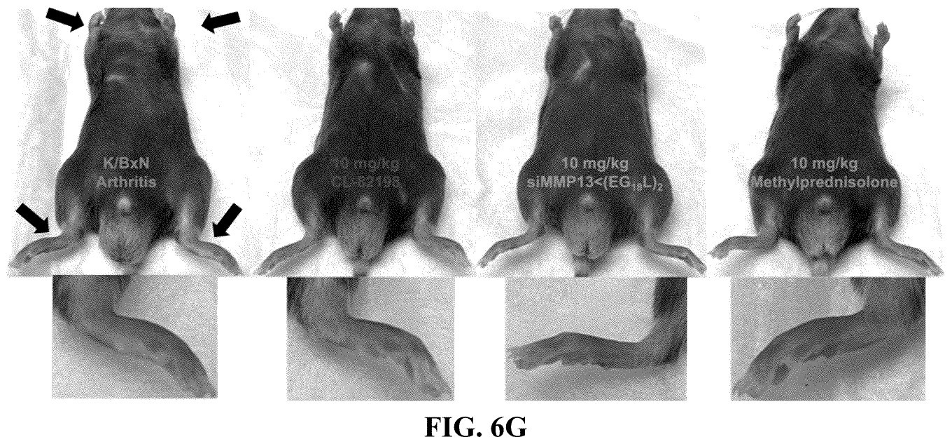

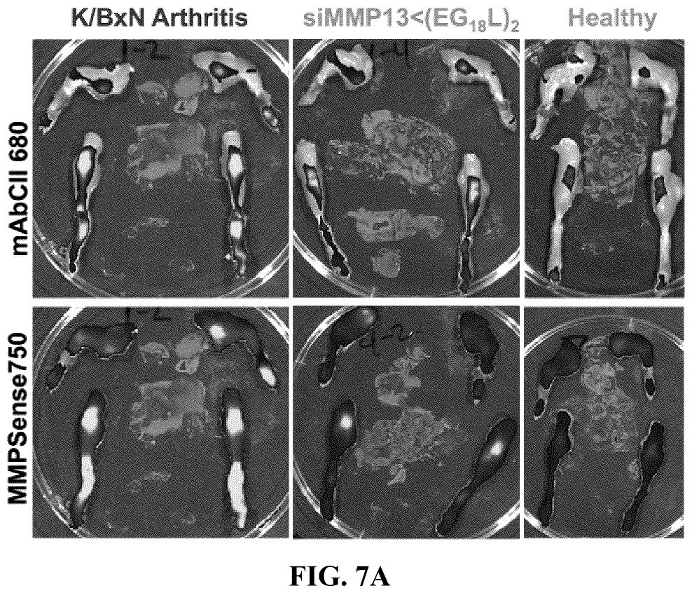

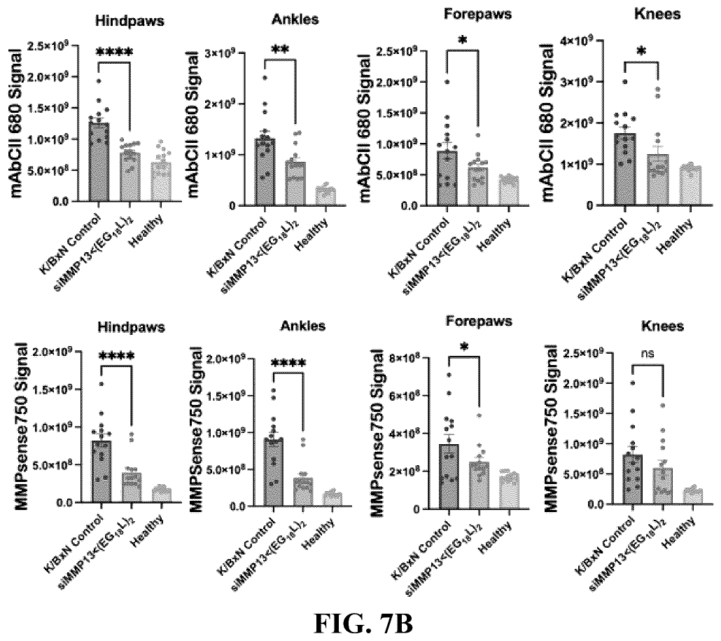

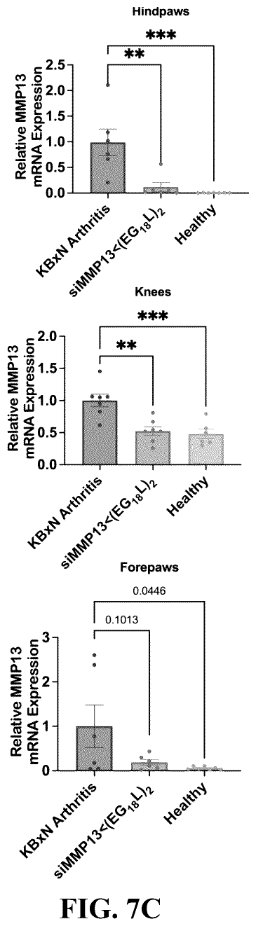

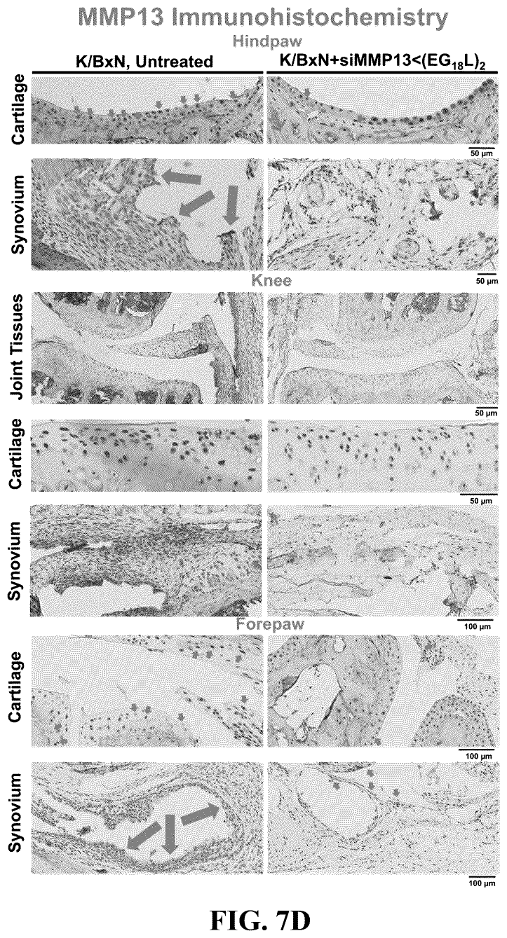

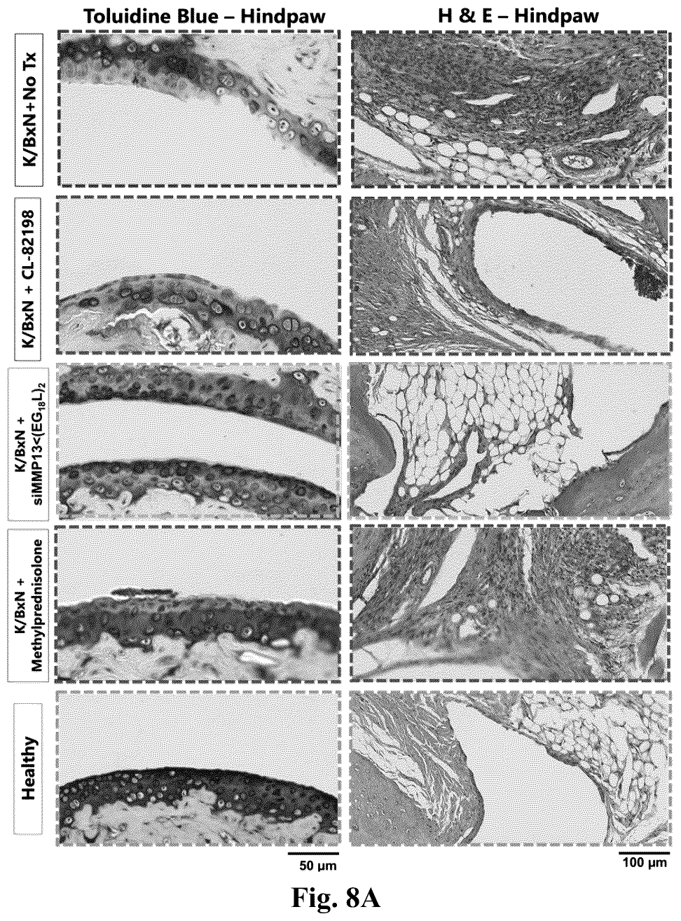

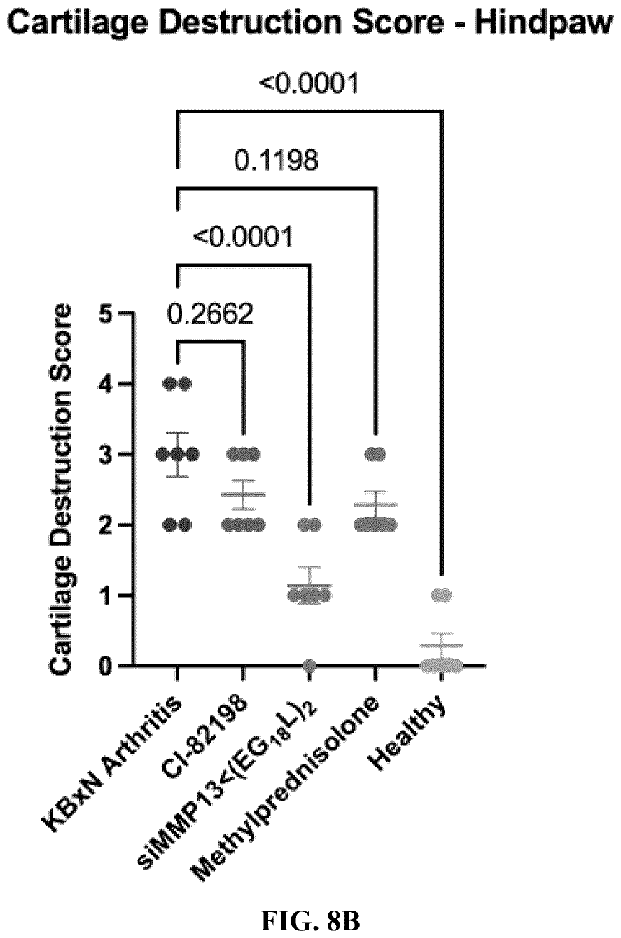

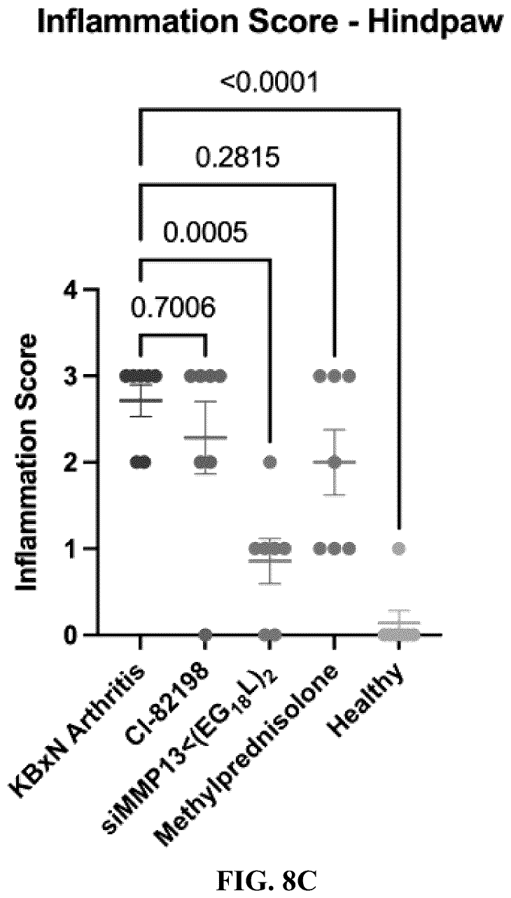

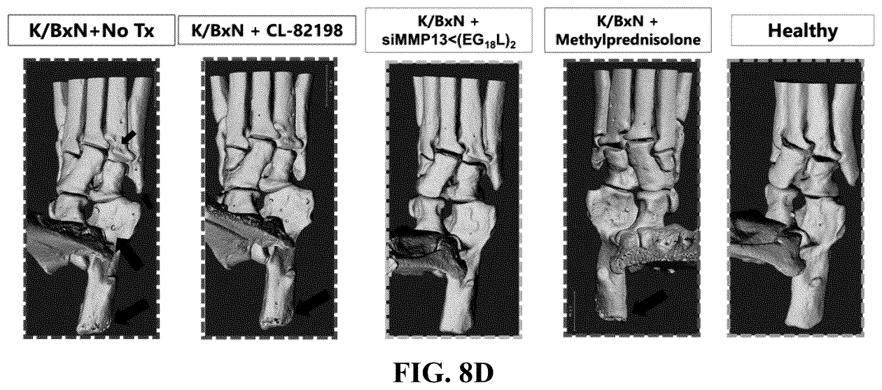

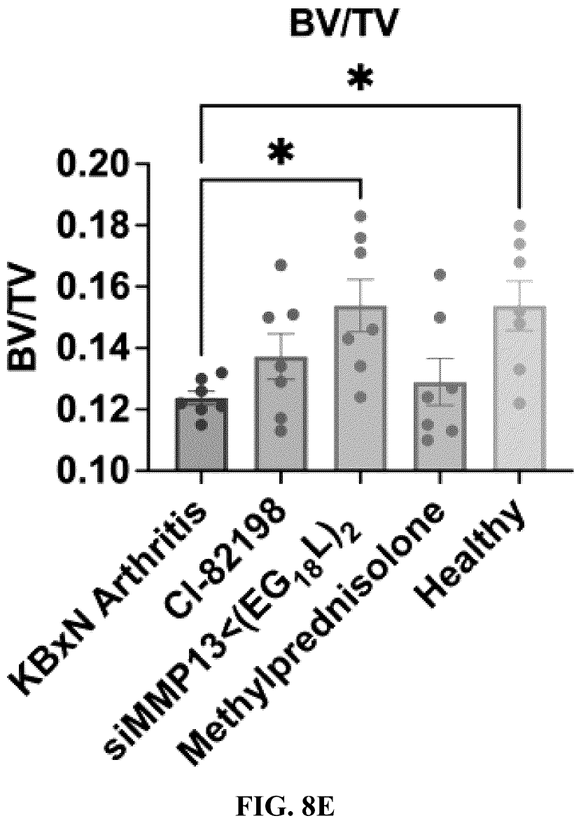

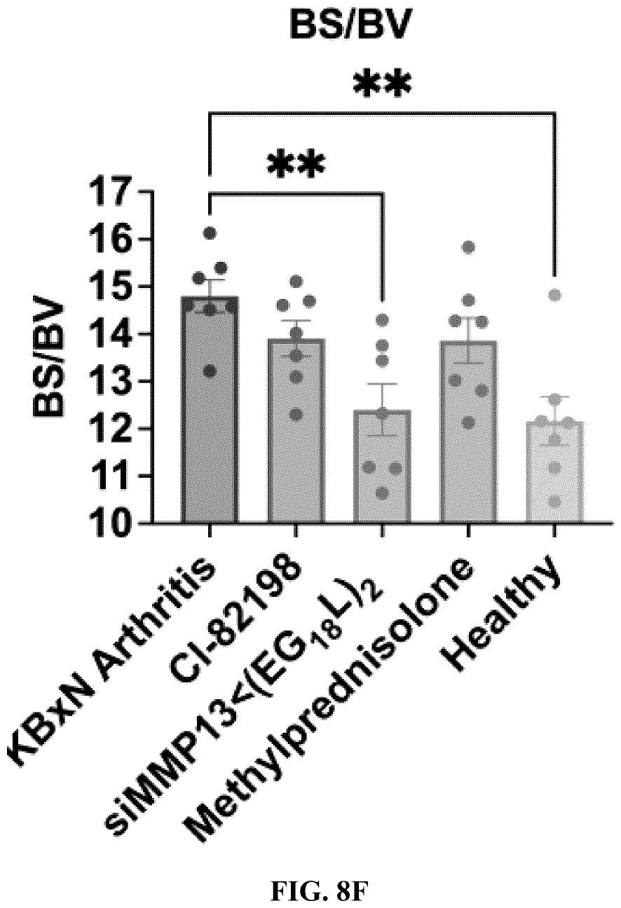

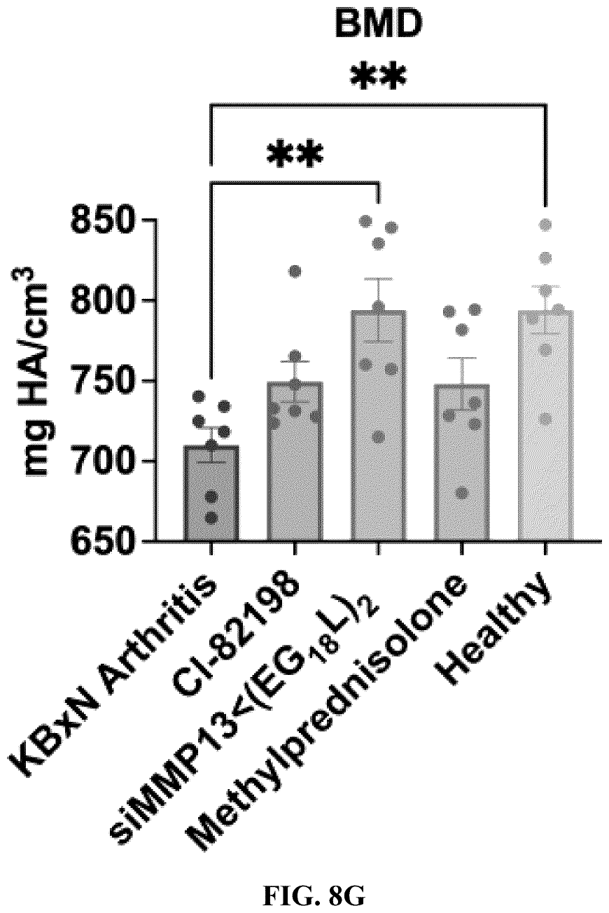

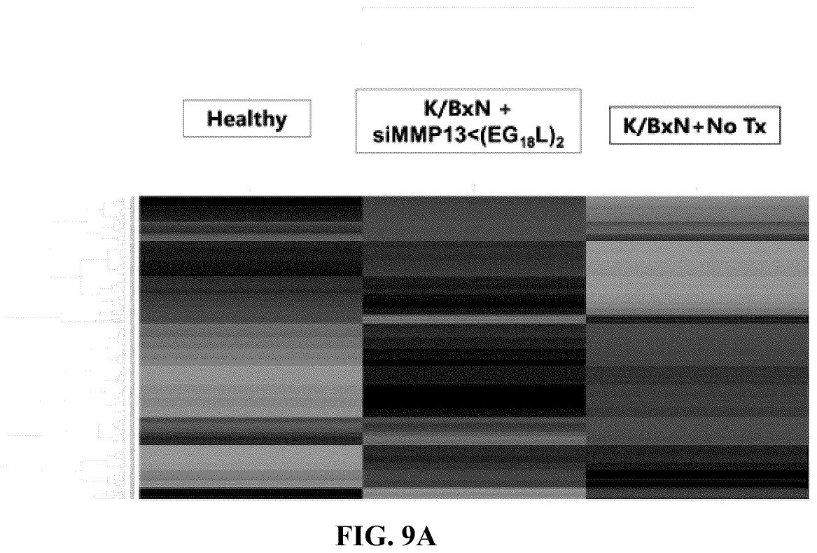

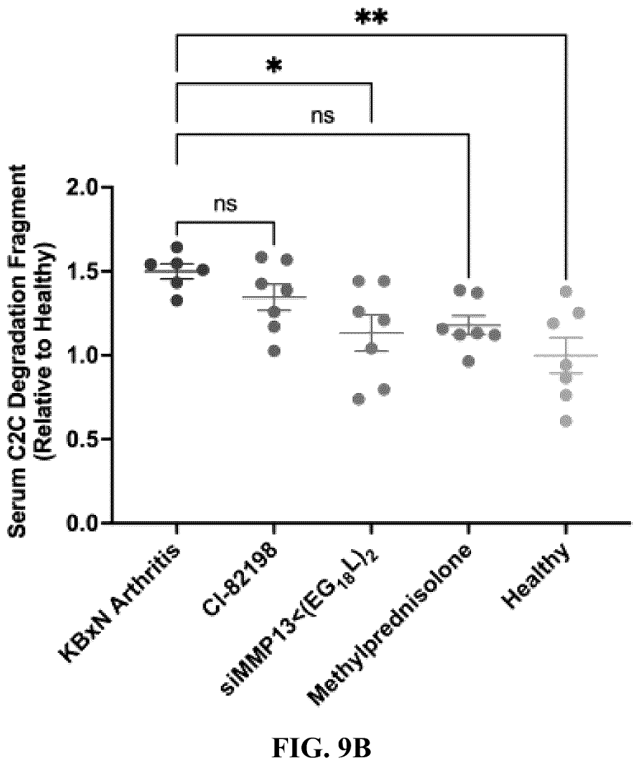

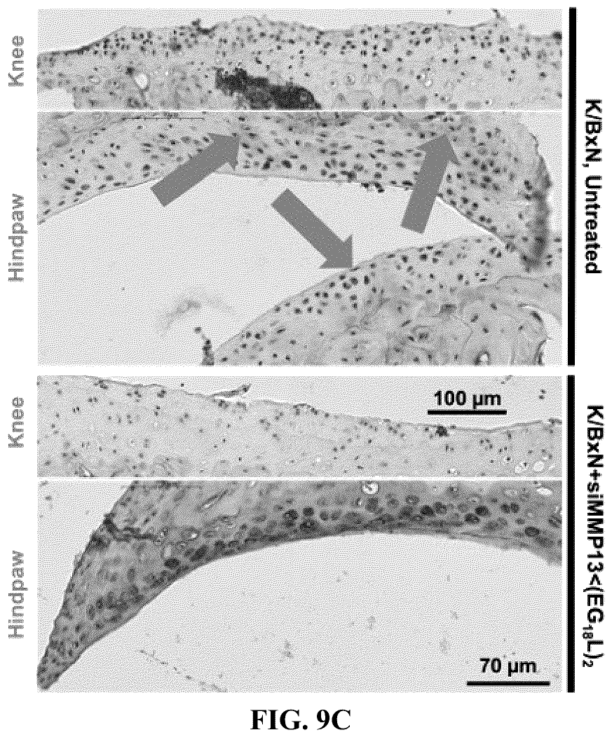

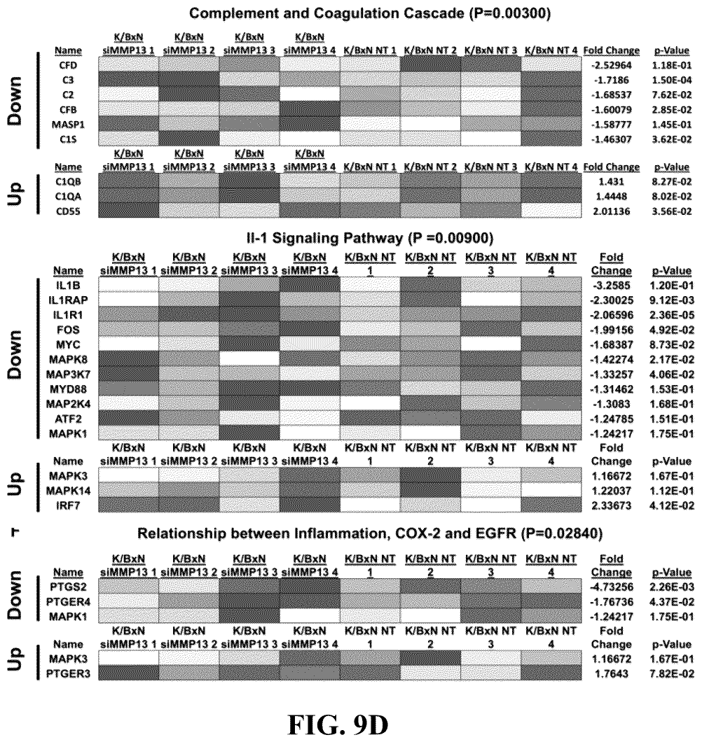

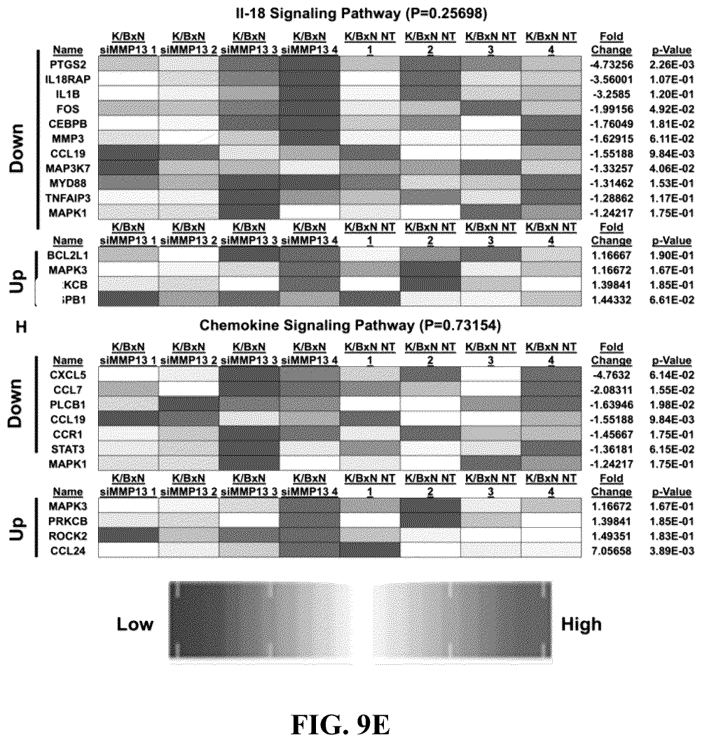

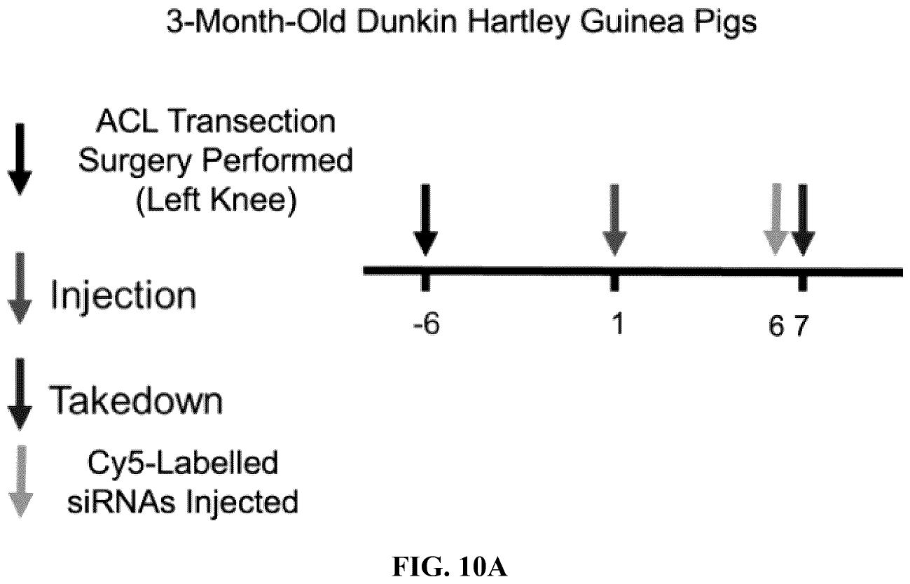

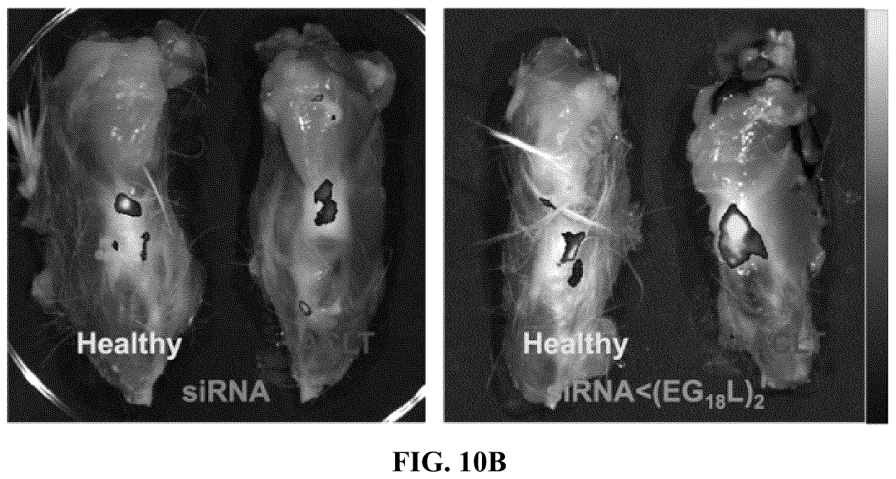

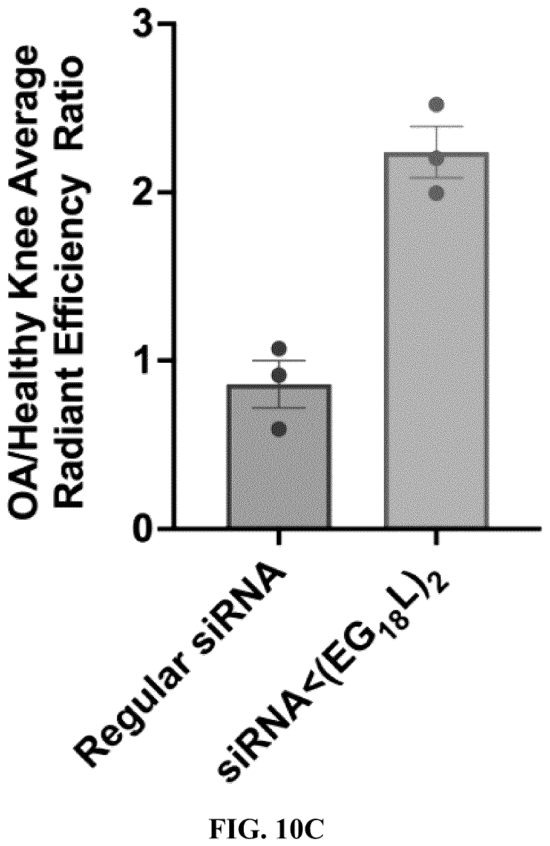

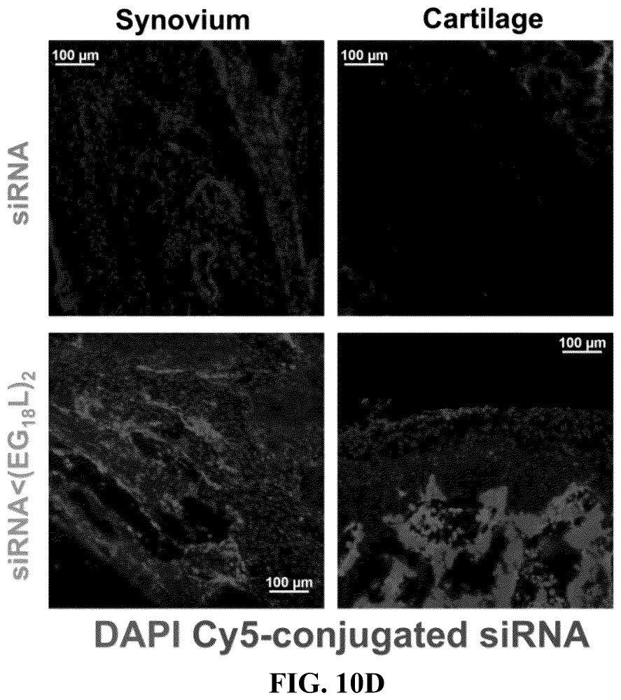

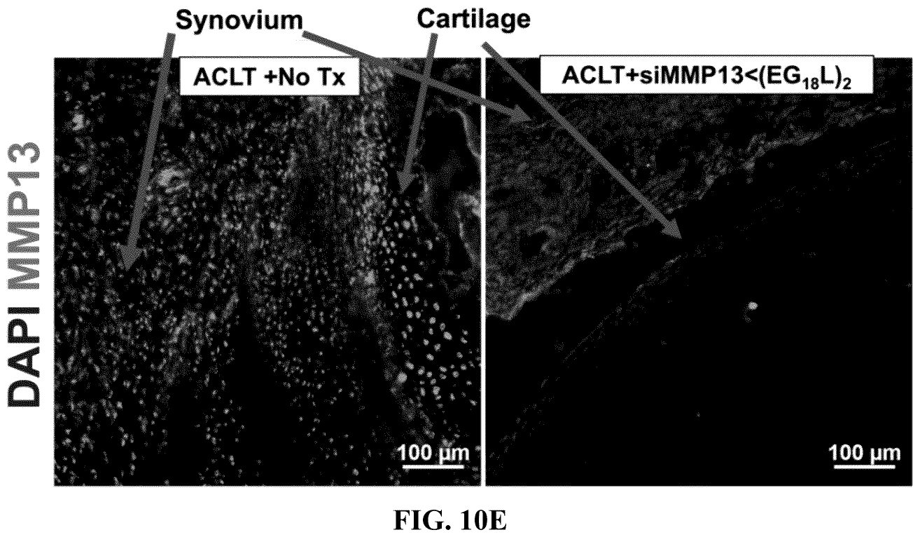

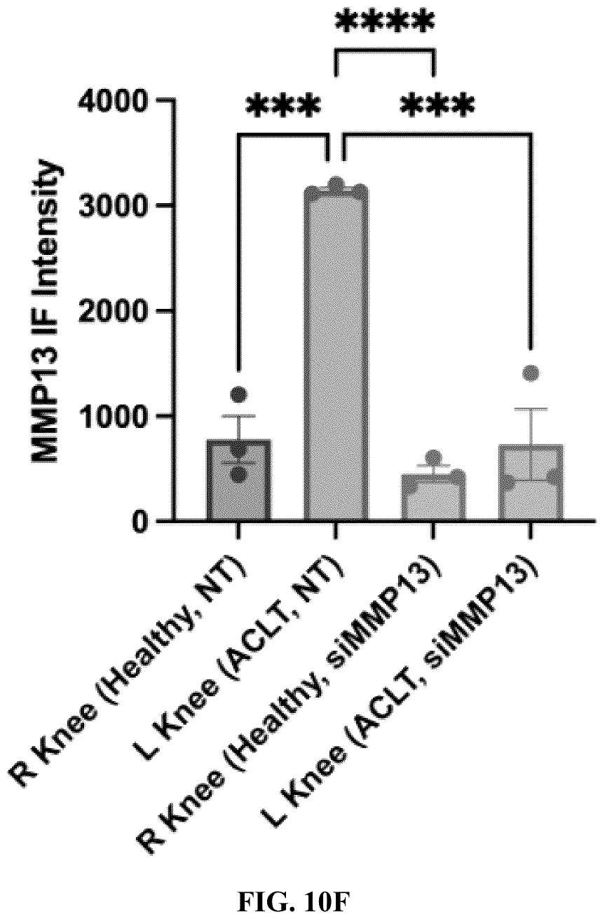

The conjugate was then assessed in vivo for different inflammatory disease models. As shown in FIG. 2A-FIG. 2I; FIG. 3A-FIG. 3G; FIG. 4A-FIG. 4G; and FIG. 5A-FIG. 5H, the conjugate demonstrated preferential accumulation, retention, and silencing of a target gene in an injured joint model of post-traumatic osteoarthritis (PTOA). The conjugate was also shown to accumulate and have activity in all joints in a serum-transfer mouse model of rheumatoid arthritis (RA) (FIG. 6A-FIG. 6G; FIG. 7A-FIG. 7D; FIG. 8A-FIG. 8G; and FIG. 9A-FIG. 9E). In both models, intravenous delivery of the siRNA conjugate yielded accumulation in the affected joint(s). Finally, the conjugate showed effective silencing in a guinea pig anterior cruciate ligament transection (ACLT) model (FIG. 10A-FIG. 10F).

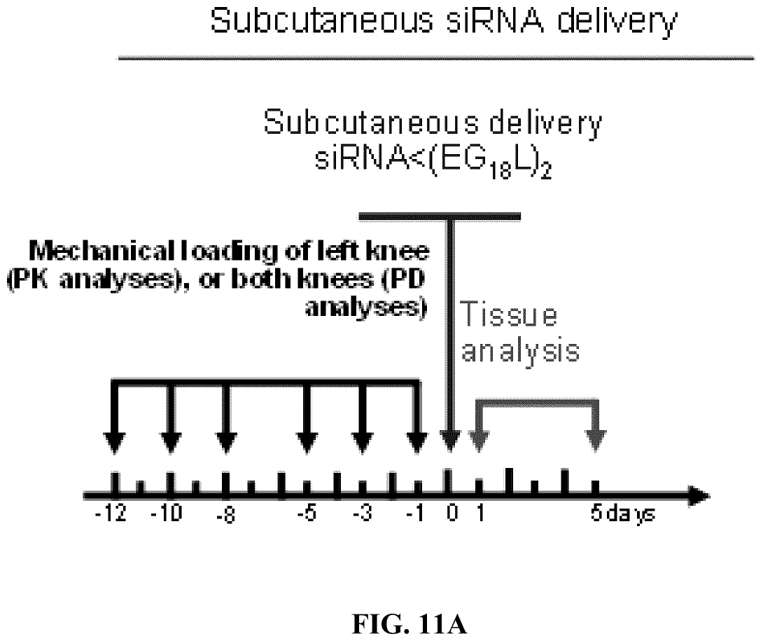

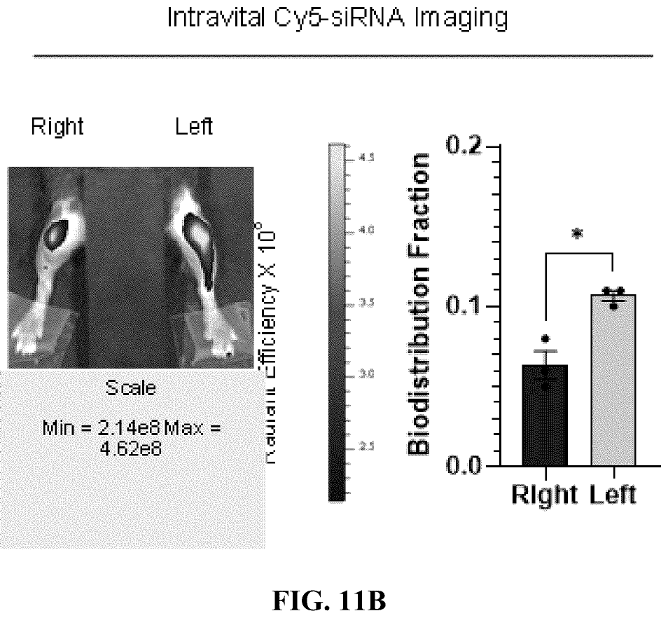

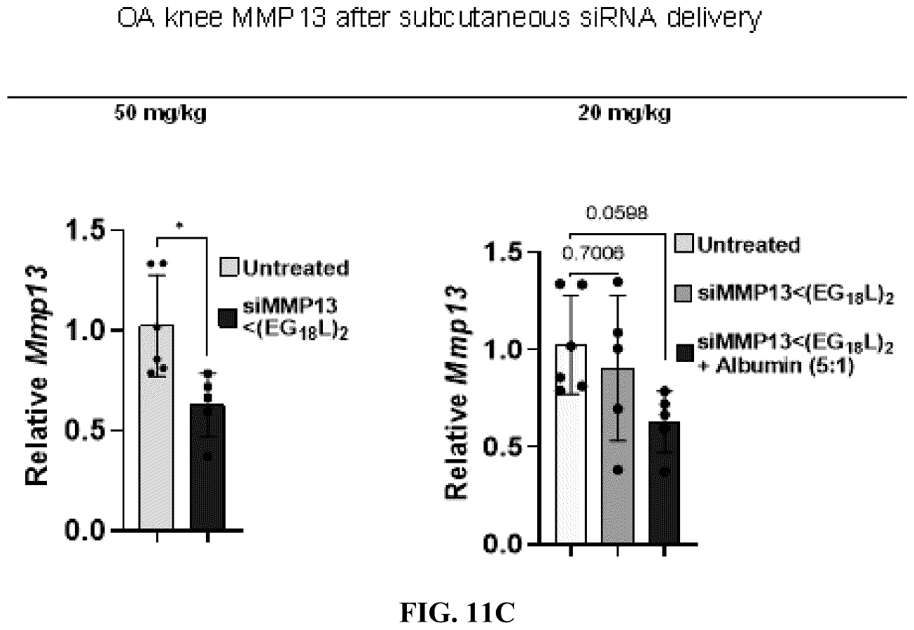

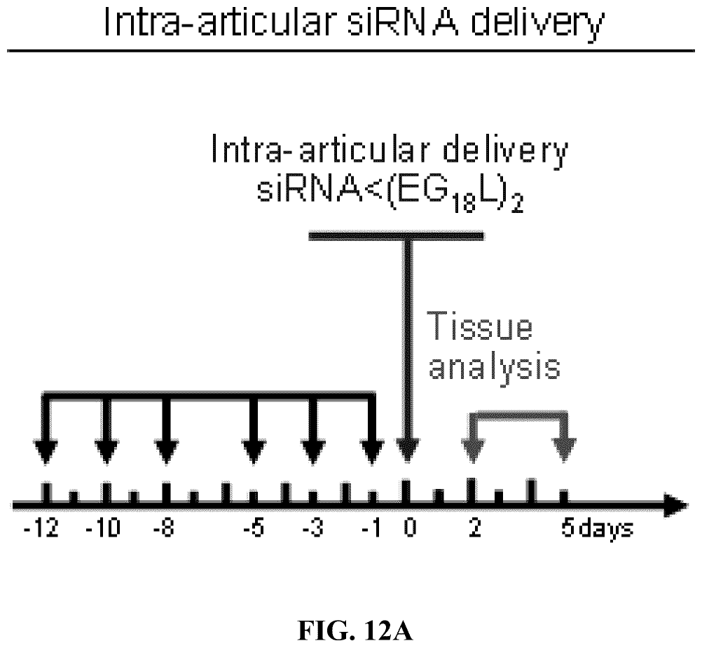

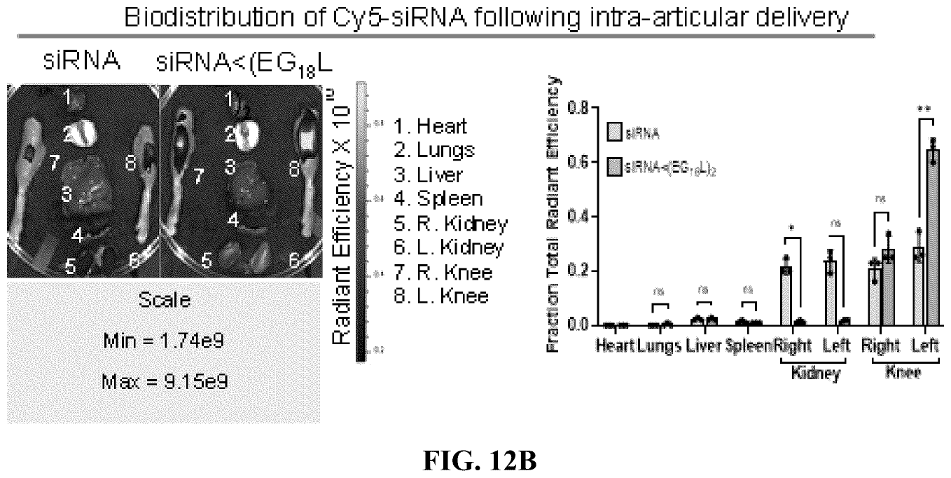

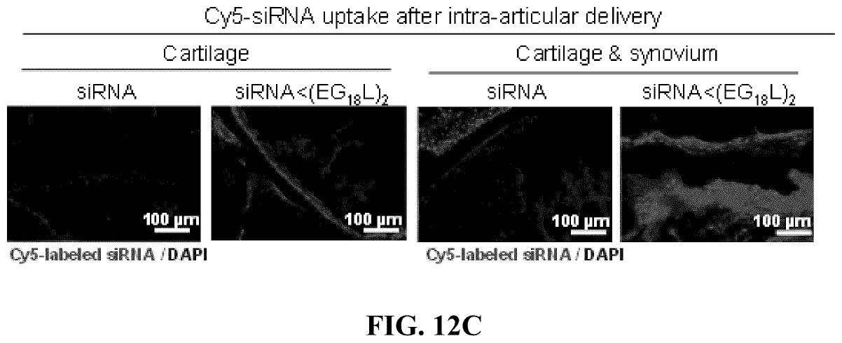

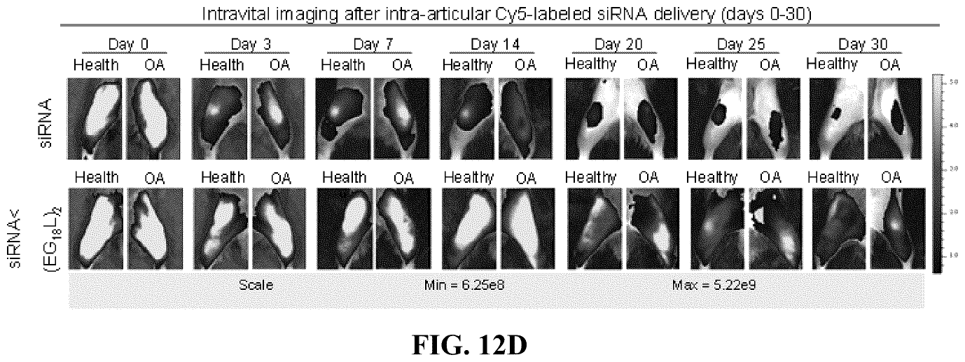

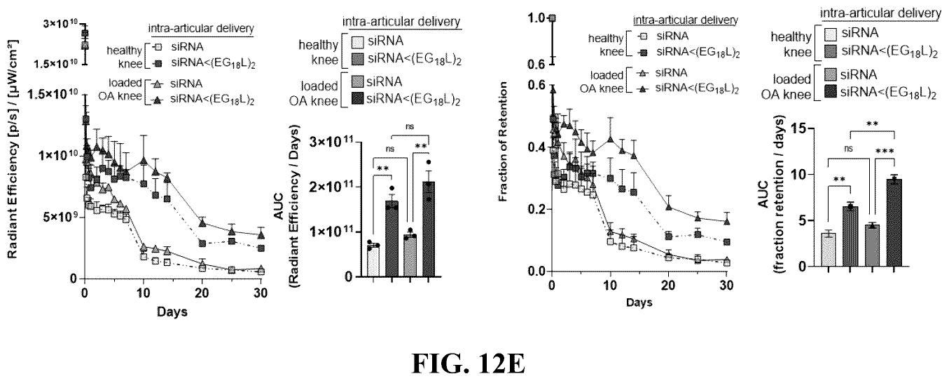

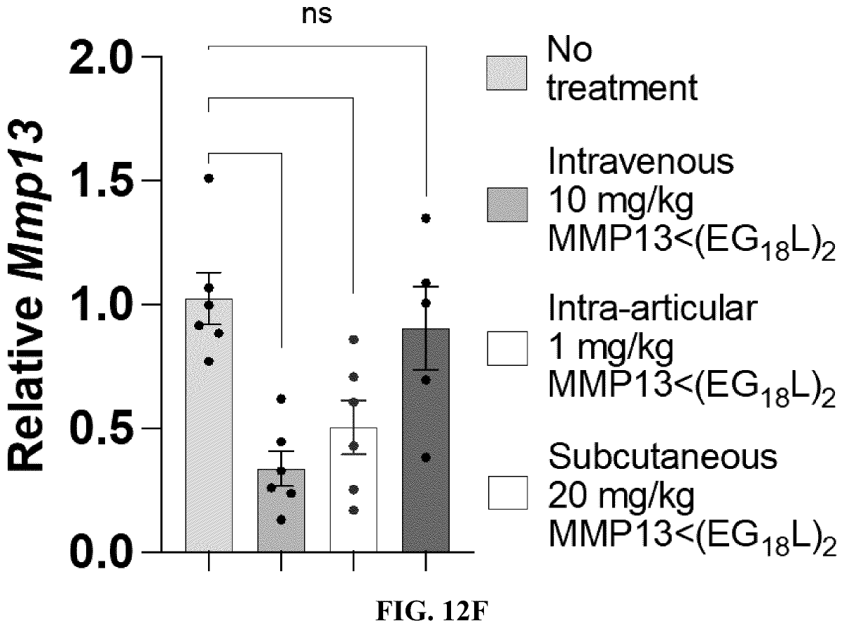

When designed against matrix metalloproteinase 13 (MMP13), the siRNA conjugate robustly silenced MMP13 expression in the joints. In both the PTOA and RA models, silencing of MMP13 by systemic, intra-venous, treatment provided significant therapeutic benefits in terms of reducing joint inflammation, reducing cartilage loss, reducing synovial hyperplasia, maintaining bone homeostasis and reducing joint pressure sensitivity (pain). In-vivo subcutaneous studies (FIG. 11A-FIG. 11C) show useful delivery and activity of siMMP13<(EG₁₈L)₂ at high doses and/or with the use of excipients. In-vivo local, intra-articular studies also show useful delivery, retention, and activity of siMMP13<(EG₁₈L)₂ (FIG. 12A-FIG. 12F).

It is understood that the foregoing detailed description and accompanying examples are merely illustrative and are not to be taken as limitations upon the scope of the invention.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:
Clause 1. A method of treating an inflammatory disease in a subject in need thereof, the method comprising administering to the subject an effective amount of a conjugate, optionally in combination with a pharmaceutically acceptable excipient, wherein the conjugate comprises a siRNA capable of inhibiting expression of a protein associated with the inflammatory disease; a lipophilic ligand capable of binding albumin; and a linker attaching the siRNA to the lipophilic ligand, the linker comprising a branching molecule attached to the siRNA, and a hydrophilic spacer attaching the branching molecule to the lipophilic ligand.
Clause 2. The method of clause 1, wherein the conjugate is administered intravenously, subcutaneously, intraarticularly, orally, by inhalation, or locally at a site of inflammation associated with the inflammatory disease.
Clause 3. The method of clause 1 or 2, wherein the conjugate localizes to a site of inflammation associated with the inflammatory disease.
Clause 4. The method of any one of clauses 1-3, wherein the method decreases the underlying pathology associated with the inflammatory disease in the subject for at least 30 days post-administration.
Clause 5. The method of any one of clauses 1-4, wherein the inflammatory disease is arthritis or an inflammatory state associated with a traumatic injury.
Clause 6. The method of any one of clauses 1-5, wherein the inflammatory disease is arthritis and is selected from the group consisting of osteoarthritis, rheumatoid arthritis, inflammatory arthritis, multi-joint arthritis, gout, and psoriatic arthritis.
Clause 7. The method of clause 6, wherein the inflammatory disease is osteoarthritis.
Clause 8. The method of clause 6 or 7, wherein the subject has decreased hyperalgesia, increased cartilage protection, decreased synovial inflammation, decreased osteophytes, decreased bone erosion, or a combination thereof in a joint associated with the inflammatory disease following administration.
Clause 9. The method of clause 8, wherein the joint is a knee, a wrist, an ankle, a shoulder, a spinal joint, or a combination thereof.
Clause 10. The method of any one of clauses 7-9, wherein the method provides increased cartilage protection compared to a steroid control.
Clause 11. The method of any one of clauses 1-10, wherein the conjugate is administered at a dosage of about 1 mg/kg to about 50 mg/kg.
Clause 12. The method of any one of clauses 1-11, wherein the conjugate is administered at least once over 30 days.
Clause 13. The method of any one of clauses 1-12, wherein the pharmaceutically acceptable excipient comprises saline, phosphate buffered saline, albumin, dimethyl sulfoxide, trehalose, sucrose, polyethylene glycol, an absorption enhancer, or a combination thereof.
Clause 14. The method of any one of clauses 1-13, wherein the subject is human.
Clause 15. The method of any one of clauses 1-14, wherein the branching molecule includes at least one branch point having at least two independent branches.
Clause 16. The method of any one of clauses 1-15, wherein the hydrophilic spacer comprises 1 to 100 hydrophilic blocks.
Clause 17. The method of clause 16, wherein each hydrophilic block comprises 1 to 150 repeats of a hydrophilic compound.
Clause 18. The method of clause 17, wherein the hydrophilic compound comprises ethylene glycol, zwitterionic linkers, peptoids, amino acids, poly(glycerols), poly(oxazoline), poly(acrylamide), poly(N-acryloyl morpholine, poly(N,N-dimethyl acrylamide), poly(2-hydroxypropyl methacrylamide), poly(2-hydroxyethyl methacryalmide), or a combination thereof.
Clause 19. The method of clause 17, wherein each hydrophilic block comprises 1 to 100 repeats of ethylene glycol.
Clause 20. The method of any one of clauses 17-19, wherein the hydrophilic blocks are attached to each other through phosphorothioate linkages.
Clause 21. The method of any one of clauses 1-20, wherein the siRNA is capable of specifically hybridizing to an oligonucleotide encoding a protein of a p38/MAPK signaling pathway, a TNF-α NF-κB signaling pathway, a chemokine signaling pathway, an IL-1 signaling pathway, a senescence-associated secretory phenotype pathway, or a combination thereof.
Clause 22. The method of any one of clauses 1-21, wherein the siRNA is capable of specifically hybridizing to an oligonucleotide encoding an extracellular matrix degrading protein.
Clause 23. The method of any one of clauses 1-22, wherein the siRNA is capable of specifically hybridizing to an oligonucleotide encoding MMP13, Cadherin-11, MMP1, SOX5, NGF, or MK2.
Clause 24. The method of any one of clauses 1-23, wherein the siRNA comprises a nucleotide sequence of SEQ ID NO: 1, SEQ ID NO: 2, SEQ ID NO: 3, SEQ ID NO: 4, or a combination thereof.
Clause 25. The method of any one of clauses 1-24, wherein the siRNA comprises stabilizing modifications.
Clause 26. The method of any one of clauses 1-25, wherein the siRNA comprises a plurality of phosphorothioate linkages.
Clause 27. The method of any one of clauses 1-26, wherein the siRNA has about 15 nucleotides to about 40 nucleotides.
Clause 28. The method of any one of clauses 1-27, wherein the lipophilic ligand comprises a lipid including a C₁₂-C₂₂ hydrocarbon chain.
Clause 29. The method of any one of clauses 1-28, wherein the lipophilic ligand is divalent.
Clause 30. The method of any one of clauses 1-29, wherein the lipophilic ligand comprises two independent lipids, each lipid including a C₁₂-C₂₂ hydrocarbon chain.
Clause 31. The method of clause 30, wherein each lipid includes a Cis hydrocarbon chain.
Clause 32. The method of clause 28, wherein the lipid includes a carboxyl at its terminal end.
Clause 33. The method of clause 15, wherein each branch is attached to an individual hydrophilic spacer, and each hydrophilic spacer is attached to an individual lipid of the lipophilic ligand.
Clause 34. The method of any one of clauses 1-33, wherein the hydrophilic spacer is attached to the lipophilic ligand through a phosphorothioate linkage.
Clause 35. The method of any one of clauses 1-34, wherein the conjugate has a binding affinity (K_{d}) to albumin of less than 1 μM.
Clause 36. The method of any one of clauses 1-35, wherein the conjugate has a binding affinity (K_{d}) to albumin of less than 100 nM.
Clause 37. The method of any one of clauses 1-36, wherein the conjugate has a critical micelle concentration of greater than 1850 nM.
Clause 38. The method of any one of clauses 1-37, wherein the conjugate comprises about 20% to about 60% phosphorothioate linkages based on a total amount of phosphate-based linkages of the conjugate.
Clause 39. The method of any one of clauses 1-38, wherein the conjugate comprises a lipophilic ligand capable of binding albumin, the lipophilic ligand comprising two independent lipids, each lipid including a C₁₈ hydrocarbon chain; and a linker attaching the siRNA to the lipophilic ligand, the linker comprising a branching molecule attached to the siRNA and including at least one branch point having at least two independent branches, and a hydrophilic spacer attaching an individual branch to an individual lipid, the hydrophilic spacer including 1 to 6 hydrophilic blocks, each hydrophilic block including 2 to 10 repeats of ethylene glycol.

Sequences
Mouse MMP13:
Antisense
(SEQ ID NO: 1): 5′(PHO)(OMeU)*(fU)*(OMeU)(fC)(OMeU)(fC)(OMeA)(fU)(OMeG)
(fA)(OMeU)(fG)(OMeU)(fC)(OMeU)(fA)(OMeA)*(fG)*(OMeG)3′
MW = 6303 g/mol
Sense
(SEQ ID NO: 2): 5′(fC)*(OMeC)*(fU)(OMeU)(fA)(OMeG)(fA)(OMeC)(fA)
(OMeU)(fC)(OMeA)(fU)(OMeG)(fA)(OMeG)(fA)*(OMeA)*(fA)3′
MW = 6263.1 g/mol
Guinea Pig MMP13:
Antisense
(SEQ ID NO: 3): 5′(PHO)(OMeA)*(fU)*(OMeG)(fC)(OMeC)(fC)(OMeG)(fC)(OMeA)
(fA)(OMeG)(fA)(OMeU)(fU)(OMeU)(fA)(OMeC)*(fU)*(OMeG)3′
MW = 6206.9 g/mol
Sense
(SEQ ID NO: 4): 5′(fC)*(OMeA)*(fG)(OMeU)(fA)(OMeA)(fA)(OMeA)(fU)
(OMeC)(fU)(OMeU)(fG)(OMeC)(fG)(OMeG)(fG)*(OMeC)*(fA)3′
MW = 6344.2 g/mol
<(EG₁₈L)₂ Modifier
MW = 3027 g/mol
Cy5 Fluorescent Modifier
MW = 533.63 g/mol
<(EG₃)Cholesterol Modifier
MW = 699 g/mol
PHO = Phosphate, * = Phosphorothioate, f = 2′fluoro, OMe = 2′O-methyl

What is claimed is:

1. A method of treating an inflammatory disease in a subject in need thereof, the method comprising administering to the subject an effective amount of a conjugate, optionally in combination with a pharmaceutically acceptable excipient, wherein the conjugate comprises
a siRNA capable of inhibiting expression of a protein associated with the inflammatory disease;
a lipophilic ligand capable of binding albumin; and
a linker attaching the siRNA to the lipophilic ligand, the linker comprising
a branching molecule attached to the siRNA, and
a hydrophilic spacer attaching the branching molecule to the lipophilic ligand.

2. The method of claim 1, wherein the conjugate is administered intravenously, subcutaneously, intraarticularly, orally, by inhalation, or locally at a site of inflammation associated with the inflammatory disease.

3. The method of claim 1, wherein the conjugate localizes to a site of inflammation associated with the inflammatory disease.

4. The method of claim 1, wherein the method decreases the underlying pathology associated with the inflammatory disease in the subject for at least 30 days post-administration.

5. The method of claim 1, wherein the inflammatory disease is arthritis or an inflammatory state associated with a traumatic injury.

6. The method of claim 5, wherein the inflammatory disease is arthritis and is selected from the group consisting of osteoarthritis, rheumatoid arthritis, inflammatory arthritis, multi-joint arthritis, gout, and psoriatic arthritis.

7. The method of claim 6, wherein the inflammatory disease is osteoarthritis.

8. The method of claim 6, wherein the subject has decreased hyperalgesia, increased cartilage protection, decreased synovial inflammation, decreased osteophytes, decreased bone erosion, or a combination thereof in a joint associated with the inflammatory disease following administration.

9. The method of claim 8, wherein the joint is a knee, a wrist, an ankle, a shoulder, a spinal joint, or a combination thereof.

10. The method of claim 7, wherein the method provides increased cartilage protection compared to a steroid control.

11. The method of claim 1, wherein the conjugate is administered at a dosage of about 1 mg/kg to about 50 mg/kg.

12. The method of claim 1, wherein the conjugate is administered at least once over 30 days.

13. The method of claim 1, wherein the pharmaceutically acceptable excipient comprises saline, phosphate buffered saline, albumin, dimethyl sulfoxide, trehalose, sucrose, polyethylene glycol, an absorption enhancer, or a combination thereof.

14. The method of claim 1, wherein the subject is human.

15. The method of claim 1, wherein the branching molecule includes at least one branch point having at least two independent branches.

16. The method of claim 1, wherein the hydrophilic spacer comprises 1 to 100 hydrophilic blocks.

17. The method of claim 16, wherein each hydrophilic block comprises 1 to 150 repeats of a hydrophilic compound.

18. The method of claim 17, wherein the hydrophilic compound comprises ethylene glycol, zwitterionic linkers, peptoids, amino acids, poly(glycerols), poly(oxazoline), poly(acrylamide), poly(N-acryloyl morpholine, poly(N,N-dimethyl acrylamide), poly(2-hydroxypropyl methacrylamide), poly(2-hydroxyethyl methacryalmide), or a combination thereof.

19. The method of claim 17, wherein each hydrophilic block comprises 1 to 100 repeats of ethylene glycol.

20. The method of claim 17, wherein the hydrophilic blocks are attached to each other through phosphorothioate linkages.

21. The method of claim 1, wherein the siRNA is capable of specifically hybridizing to an oligonucleotide encoding a protein of a p38/MAPK signaling pathway, a TNF-α NF-κB signaling pathway, a chemokine signaling pathway, an IL-1 signaling pathway, a senescence-associated secretory phenotype pathway, or a combination thereof.

22. The method of claim 21, wherein the siRNA is capable of specifically hybridizing to an oligonucleotide encoding an extracellular matrix degrading protein.

23. The method of claim 21, wherein the siRNA is capable of specifically hybridizing to an oligonucleotide encoding MMP13, Cadherin-11, MMP1, SOX5, NGF, or MK2.

24. The method of claim 1, wherein the siRNA comprises a nucleotide sequence of SEQ ID NO: 1, SEQ ID NO: 2, SEQ ID NO: 3, SEQ ID NO: 4, or a combination thereof.

25. The method of claim 1, wherein the siRNA comprises stabilizing modifications.

26. The method of claim 1, wherein the siRNA comprises a plurality of phosphorothioate linkages.

27. The method of claim 1, wherein the siRNA has about 15 nucleotides to about 40 nucleotides.

28. The method of claim 1, wherein the lipophilic ligand comprises a lipid including a C₁₂-C₂₂ hydrocarbon chain.

29. The method of claim 1, wherein the lipophilic ligand is divalent.

30. The method of claim 1, wherein the lipophilic ligand comprises two independent lipids, each lipid including a C₁₂-C₂ hydrocarbon chain.

31. The method of claim 30, wherein each lipid includes a C₁₈ hydrocarbon chain.

32. The method of claim 28, wherein the lipid includes a carboxyl at its terminal end.

33. The method of claim 15, wherein each branch is attached to an individual hydrophilic spacer, and each hydrophilic spacer is attached to an individual lipid of the lipophilic ligand.

34. The method of claim 1, wherein the hydrophilic spacer is attached to the lipophilic ligand through a phosphorothioate linkage.

35. The method of claim 1, wherein the conjugate has a binding affinity (K_{d}) to albumin of less than 1 μM.

36. The method of claim 1, wherein the conjugate has a binding affinity (K_{d}) to albumin of less than 100 nM.

37. The method of claim 1, wherein the conjugate has a critical micelle concentration of greater than 1850 nM.

38. The method of claim 1, wherein the conjugate comprises about 20% to about 60% phosphorothioate linkages based on a total amount of phosphate-based linkages of the conjugate.

39. The method of claim 1, wherein the conjugate comprises
a lipophilic ligand capable of binding albumin, the lipophilic ligand comprising two independent lipids, each lipid including a C₁₈ hydrocarbon chain; and
a linker attaching the siRNA to the lipophilic ligand, the linker comprising
a branching molecule attached to the siRNA and including at least one branch point having at least two independent branches, and
a hydrophilic spacer attaching an individual branch to an individual lipid, the hydrophilic spacer including 1 to 6 hydrophilic blocks, each hydrophilic block including 2 to 10 repeats of ethylene glycol.


SEQUENCE LISTING (4 sequences)

SEQ ID NO: 1 - RNA, 19 nt, synthetic construct
  source 1..19: mol_type = other RNA
  modified_base 1: mod_base = OTHER; note = '5-phosphate
  modified_base 1^2: mod_base = OTHER; note = 5'-3' phosphorothioate linkage
  modified_base 2^3: mod_base = OTHER; note = 5'-3' phosphorothioate linkage
  modified_base 17^18: mod_base = OTHER; note = 5'-3' phosphorothioate linkage
  modified_base 18^19: mod_base = OTHER; note = 5'-3' phosphorothioate linkage
  modified_base order(1,3,5,7,9,11,13,15,17,19): mod_base = OTHER; note = 2'-O-methyl modifications
  modified_base order(2,4,6,8,10,12,14,16,18): mod_base = OTHER; note = 2'-fluro modifications
tttctcatgatgtctaagg

SEQ ID NO: 2 - RNA, 19 nt, synthetic construct
  source 1..19: mol_type = other RNA
  modified_base 1^2: mod_base = OTHER; note = 5'-3' phosphorothioate linkage
  modified_base 2^3: mod_base = OTHER; note = 5'-3' phosphorothioate linkage
  modified_base 17^18: mod_base = OTHER; note = 5'-3' phosphorothioate linkage
  modified_base 18^19: mod_base = OTHER; note = 5'-3' phosphorothioate linkage
  modified_base order(1,3,5,7,9,11,13,15,17,19): mod_base = OTHER; note = 2'-fluro modifications
  modified_base order(2,4,6,8,10,12,14,16,18): mod_base = OTHER; note = 2'-O-methyl modifications
ccttagacatcatgagaaa

SEQ ID NO: 3 - RNA, 19 nt, synthetic construct
  source 1..19: mol_type = other RNA
  modified_base 1: mod_base = OTHER; note = 5'-phosphate
  modified_base 1^2: mod_base = OTHER; note = 5'-3' phosphorothioate linkage
  modified_base 2^3: mod_base = OTHER; note = 5'-3' phosphorothioate linkage
  modified_base 17^18: mod_base = OTHER; note = 5'-3' phosphorothioate linkage
  modified_base 18^19: mod_base = OTHER; note = 5'-3' phosphorothioate linkage
  modified_base order(1,3,5,7,9,11,13,15,17,19): mod_base = OTHER; note = 2'-O-methyl modifications
  modified_base order(2,4,6,8,10,12,14,16,18): mod_base = OTHER; note = 2'-fluro modifications
atgcccgcaagatttactg

SEQ ID NO: 4 - RNA, 19 nt, synthetic construct
  source 1..19: mol_type = other RNA
  modified_base 1^2: mod_base = OTHER; note = 5'-3' phosphorothioate linkage
  modified_base 2^3: mod_base = OTHER; note = 5'-3' phosphorothioate linkage
  modified_base 17^18: mod_base = OTHER; note = 5'-3' phosphorothioate linkage
  modified_base 18^19: mod_base = OTHER; note = 5'-3' phosphorothioate linkage
  modified_base order(1,3,5,7,9,11,13,15,17,19): mod_base = OTHER; note = 2'-fluro modifications
  modified_base order(2,4,6,8,10,12,14,16,18): mod_base = OTHER; note = 2'-O-methyl modifications
cagtaaaatcttgcgggca